(19) United States
(12) Patent Application Publication
(10) Pub. No.: US 2016/0287692 A1
(43) Pub. Date: Oct. 6, 2016

(54) SWINE INFLUENZA HEMAGGLUTININ AND NEURAMINIDASE VARIANTS

(71) Applicant: MEDIMMUNE, LLC, Gaithersburg, MD (US)

(72) Inventors: Hong Jin, Cupertino, CA (US); Zhongying Chen, Cupertino, CA (US)

(21) Appl. No.: 14/442,167

(22) PCT Filed: Nov. 15, 2013
(86) PCT No.: PCT/US13/70336

§ 371 Date: May 12, 2015

Related U.S. Application Data
(60) Provisional application No. 61/727,213, filed on Nov. 16, 2012.

(51) Int. Cl. A61K 39/145; C12N 7/00

(52) U.S. Cl. CPC A61K 39/145; C12N 7/00; A61K 2039/5254

(57) ABSTRACT

Polypeptides, polynucleotides, methods, compositions, and vaccines comprising influenza hemagglutinin and neuraminidase variants are provided.

### CROSS-REFERENCE TO RELATED APPLICATIONS

This application claims priority to U.S. Application No. 61/727,213 filed Nov. 16, 2012. The disclosure of this provisional application is incorporated herein by reference in its entirety.

### BACKGROUND OF THE INVENTION

The 2009 influenza pandemic, caused by swine-origin H1N1 influenza viruses, spread to over 215 countries and was responsible for at least 18,000 laboratory-confirmed deaths (Garten et al., 2009, Science 325:197-201, 31). In the event of such a pandemic, the rapid manufacture of vaccines is essential. However, growth of human influenza viruses in embryonated chicken eggs, the substrate for influenza vaccine virus production, is typically hampered by the virus' preference to bind to human over avian receptors. Egg adaptation is therefore usually required to improve vaccine virus growth in eggs (Gambaryan et al., 1989 p. 175-218. In R. Krug (ed.), The Influenza Viruses. Plenum Press, New York; Robertson 1993 Reviews in Med. Virol. 3:97-106; Robertson et al., 1987 Virol. 160:31-37; Rogers et al., 1983 Nature 304:76-78). At the onset of the H1N1 pandemic in April 2009, the development of the H1N1pdm vaccine had been hampered by such poor virus growth on eggs (Robertson et al., 2011 Vaccine 29:1836-1843).

To produce a live attenuated influenza vaccine (LAIV) against the swine-origin H1N1 influenza virus, three residues (K119E, A186D, D222G, H1 numbering throughout) in the HA protein were changed. These changes resulted in LAIV being the first H1N1pdm vaccine available in the US market. LAW has been licensed in the United States since 2003 and has been approved in other countries including South Korea and Canada (Ambrose et al., 2008 Influenza Other Respi Viruses 2:193-202). Each LAW virus is a 6:2 reassortant that contains 6 internal protein gene segments from a master donor virus that confers temperature-sensitive (ts), cold-adapted (ca) and attenuation (att) phenotypes, and antigenic hemagglutinin (HA) and neuraminidase (NA) surface glycoprotein gene segments from wild type virus (Murphy et al., 2002 Viral Immunol 15:295-323).

A/California/7/2009 (CA/09)-like H1N1pdm viruses have been circulating since 2009 and have replaced seasonal H1N1 viruses as the H1N1 strain present in annual influenza vaccines. Although currently circulating H1N1 viruses are antigenically similar to CA/09, CA/09 genetic diversity and subgroups within CA/09 have been identified among new H1N1pdm strains (CDC communication). Further, data obtained from animal models demonstrated that the emergence of a more virulent H1N1pdm was possible through sequence changes or reassortment with other influenza viruses (Ilyshina et al., 2010 mBio 1:e00249-10; Schrauwen et al., 2011 Emerging Infectious Diseases 17; Ye et al., 2010 PLoS Pathog. 6:e1001145). It is thus important to identify genetic signatures in H1N1pdm viruses that could facilitate rapid production of high-yield virus in eggs.

Like the influenza HA surface protein, the NA surface glycoprotein plays an important role in virus replication. HA binds to sialic acid receptors on the cell surface and mediates virus attachment and membrane fusion during virus entry (Skehel et al., 2000 Annu Rev Biochem. 69:531-569). NA catalyzes the removal of terminal sialic acid on the cell surface such that the newly assembled viruses could be released from the infected cells and spread (Colman et al., 1989 p. 175-218. In R. Krug (ed.), The Influenza Viruses. Plenum Press, New York). Both the HA and NA proteins recognize sialosides but with counteracting functions. Therefore, the functional balance between the receptor binding of the HA and the receptor destroying property of the NA is critical for efficient viral replication (Mitnaul et al., 2000 J Virol. 74:6015-6020; Wagner et al., 2002 Rev. Med. Virol. 12:159-166). For example, it has been shown that replication of influenza A/Fujian/411/2002 (H3N2) in eggs and MDCK cells can be improved by either changing two HA residues to increase the receptor-binding ability of the HA or by changing two NA residues to lower the enzymatic activity of the NA (Lu et al., 2005 J. Virol. 79:6763-6771). In addition, HA-NA balance and NA activity has been reported to affect H1N1pdm virus transmissibility (Lakdawala et al., 2011 PLoS Pathog. 7:e1002443; Yen et al., 2011 Proc. Natl. Acad. Sci. U.S.A. 108:14264-14269). Reports from Xu et al. using glycan binding and NA activity assays showed that the functional balance of the HA and NA activities is important for the emergence of H1N1pdm viruses (Xu et al., 2012 Functional Balance of the Hemagglutinin and Neuraminidase Activities Accompanies the Emergence of the 2009 H1N1 Influenza Pandemic. J. Virol. 86:17 9221-9232; Epub ahead of print 20 Jun. 2012 doi:10.1128/JVI.00697-12).

The present disclosure provides additional critical residues in both HA and NA of H1N1 viruses that improve vaccine virus growth in eggs. Specifically, the disclosure provides for several acidic residues in the HA globular head as well as NA residues that improve virus replication. These amino acid substitutions do not affect virus antigenicity and are suitable for vaccine production. The identification of such amino acid residues in influenza HA and NA polypeptides should assist vaccine manufacturers in the production of high yield reassortant vaccine viruses against future drifted H1N1pdm-like viruses. Numerous other benefits will become apparent upon review of the disclosure.

### SUMMARY OF THE INVENTION

The present disclosure provides a reassortant influenza virus comprising a first genome segment encoding a hemagglutinin polypeptide, wherein the hemagglutinin polypeptide comprises the amino acid sequence as shown in SEQ ID NO:1, SEQ ID NO:3, or SEQ ID NO:4.

The disclosure also provides methods of increasing replication capacity of influenza A virus in embryonated eggs by altering one or more hemagglutinin amino acid residues corresponding to amino acid residue positions 125, 127, and 209 (H1 numbering) to a non-naturally occurring acidic amino acid residue.

The disclosure further provides methods of increasing replication capacity of influenza A virus in embryonated eggs by altering one or more neuraminidase amino acid residues corresponding to amino acid residue positions 222, 241, and 369 (N1 numbering) to a non-naturally occurring amino acid residue.

Furthermore, the disclosure provides isolated hemagglutinin polypeptides and isolated neuraminidase polypeptides. Isolated hemagglutinin polypeptides may comprise the amino acid sequence as shown in SEQ ID NO:1, SEQ ID NO:3, or SEQ ID NO:4. Isolated neuraminidase polypeptide may comprise the amino acid sequence as shown in SEQ ID NO:5, SEQ ID NO:7, or SEQ ID NO:8.

### BRIEF DESCRIPTION OF THE FIGURES

FIG. 1. The different growth of H1N1pdm ca viruses in eggs. FIG. 1A. Depicts virus titers in eggs. Briefly, 6:2 ca reassortants with HA and NA gene segments from A/Brisbane/10/2010 (Bris/10), A/New Hampshire/2/2010 (NH/10) or A/Gilroy/231/2011 (Gil/11) were inoculated into eggs and the infectious titers were determined by FFA. The amino acid changes in the HA protein caused by egg adaptations were indicated. The data represented the average of three independent experiments with the standard deviation bar indicated. The limit of detection is 3.2 Log₁₀) FFU/ml. FIG. 1B. Depicts images of Bris/10 ca and NH/10 ca viruses containing the indicated HA amino acid changes and grown in MDCK cells. Plaque assay was performed in MDCK cells and the plaques were immunostained with polyclonal antiserum against influenza A viruses.

FIG. 2. HA sequence changes at 125, 127 and 209 improve the growth of CA/09 ca virus in eggs. FIG. 2A. Depicts virus titers in eggs. Briefly, CA/09 ca reassortants with the indicated amino acid changes in the HA gene were inoculated into eggs and the infectious titers were determined by FFA. The data represented the average of three independent experiments with the standard deviation bar indicated. The limit of detection is 3.2 log₁₀ FFU/ml. FIG. 2B. Depicts images of the CA/09 ca variants containing the indicated HA amino acid changes and grown in MDCK cells. Plaque assay was performed in MDCK cells and the plaques were immunostained with polyclonal antiserum against influenza A viruses.

FIG. 3. The effect of NA segment on the Gil/11 ca virus growth in eggs. FIG. 3A. Depicts virus titers in eggs. Briefly, the 6:2 ca reassortants containing the Gil/11 HA variants with the indicated amino acid changes and the NA segment from either Gil/11 or Bris/10 were rescued by reverse genetics. The viruses were inoculated into eggs and the infectious titers were determined by FFA. The data represented the average of three independent experiments with the standard deviation bar indicated. The limit of detection is 3.2 log₁₀ FFU/ml. FIG. 3B. Depicts images of the viruses described in FIG. 3A when grown in MDCK cells. Plaque assay was performed in MDCK cells and the plaques were immunostained with polyclonal antiserum against influenza A viruses.

FIG. 4. The effect of NA residues on the Gil/11 ca virus growth in eggs. FIG. 4A. Depicts virus titers in eggs. Briefly, the Gil/11 ca reassortants containing N25D/D127E changes in HA and the indicated amino acid changes in NA were inoculated into eggs and the infectious titers were determined by FFA. The data represented the average of three independent experiments with the standard deviation bar indicated. The limit of detection is 3.2 log₁₀ FFU/ml. FIG. 4B. Depicts images of the above described Gil/11 ca variants when grown in MDCK cells. Plaque assay was performed in MDCK cells and the plaques were immunostained with polyclonal antiserum against influenza A viruses.

FIG. 5. FIG. 5A. Depicts Growth kinetics of the 6:2 ca reassortants CA/09-D127E and CA/09-N125D/D127E in MDCK cells. MDCK cells were infected with the two viruses at an MOI of 5 or 0.005 and incubated at 33° C. At the indicated time intervals, the culture supernatants were collected and the virus titer was determined by FFA assay in MDCK cells. FIG. 5B. Depicts an image of a western blot of proteins obtained from cell lysates or supernatants of viruses grown in MDCK cells. Briefly, MDCK cells were infected with the two viruses at an MOI of 5 and incubated at 33° C. The infected cell supernatants and cell lystates were harvested after 8 hrs or 16 hrs of postinfection and analyzed by western blotting using a polyclonal antibody against H1N1pdm HA. FIG. 5C. Depicts immunostained images of MDCK cells infected with the two viruses at an MOI of 0.005 and incubated at 33° C. At 15 hrs or 48 hrs of postinfection the infected cell monolayers were immunostained with a polyclonal antibody against H1N1pdm HA.

FIG. 6. Depicts an image of a western blot of proteins obtained from cell lysates or supernatants of viruses grown in MDCK cells. Viral protein expression and release from infected cells. MDCK cells were infected with Gil/11-N125D/D127E ca viruses containing Gil/11 NA or Bris/10 NA at an MOI of 5 and incubated at 33° C. The infected cell supernatants and cell lystates were harvested after 8 hrs or 16 hrs of postinfection and analyzed by western blotting using a polyclonal antibody against H1N1pdm HA.

FIG. 7. Crystal structure of the HA and NA. FIG. 7A. Depicts an image of the crystal structure of HA. The location of the identified HA residues that improve the growth of H1N1pdm viruses on the HA 3D structure (only one monomer shown) are identified. FIG. 7B. Depicts an image of the crystal structure of NA. The locations of the three identified NA residues on one NA monomer structure are identified. HA structure: PDB#3LZG; NA structure: PDB#3NSS. The pictures were shown by using the PyMoL software. RBS: receptor binding site; AC: NA activity cavity.

### DETAILED DESCRIPTION

It should be appreciated that the particular implementations shown and described herein are examples, and are not intended to otherwise limit the scope of the application in any way. It should also be appreciated that each of the embodiments and features described herein can be combined in any and all ways.

The published patents, patent applications, websites, company names, and scientific literature referred to herein are hereby incorporated by reference in their entirety to the same extent as if each was specifically and individually indicated to be incorporated by reference. Any conflict between any references cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As used herein, the singular forms “a,” “an” and “the” specifically also encompass the plural forms of the terms to which they refer, unless the content clearly dictates otherwise.

Technical and scientific terms used herein have the meaning commonly understood by one of skill in the art to which the present application pertains, unless otherwise defined. Reference is made herein to various methodologies and materials known to those of skill in the art. Standard reference works setting forth the general principles of recombinant DNA technology include Sambrook et al., “Molecular Cloning: A Laboratory Manual,” 2nd Ed., Cold Spring Harbor Laboratory Press, New York (1989); Kaufman et al., Eds., “Handbook of Molecular and Cellular Methods in Biology in Medicine,” CRC Press, Boca Raton (1995); and McPherson, Ed., “Directed Mutagenesis: A Practical Approach,” IRL Press, Oxford (1991), the disclosures of each of which are incorporated by reference herein in their entireties.

### Reassortant Influenza Viruses

In general, influenza viruses, whether found in nature or produced via manipulation by man, are made up of an internal ribonucleoprotein core containing a segmented single-stranded RNA genome and an outer lipoprotein envelope lined by a matrix protein. The genome of influenza viruses is composed of eight segments of linear (−) strand ribonucleic acid (RNA), encoding the immunogenic surface hemagglutinin (HA) and neuraminidase (NA) proteins, and six internal core polypeptides: the nucleocapsid nucleoprotein (NP); matrix proteins (M); non-structural proteins (NS); and 3 RNA polymerase (PA, PB1, PB2) proteins. During replication, the genomic viral RNA is transcribed into (+) strand messenger RNA and (−) strand genomic cRNA in the nucleus of the host cell. Each of the eight genomic segments is packaged into ribonucleoprotein complexes that contain, in addition to the RNA, NP and a polymerase complex (PB1, PB2, and PA).

Influenza types A and B are typically associated with influenza outbreaks in human populations. However, type A influenza also infects other species as well, e.g., birds, pigs, and other animals. The type A viruses are categorized into subtypes based upon differences within their hemagglutinin and neuraminidase surface glycoprotein antigens. Hemagglutinin in type A viruses has 16 known subtypes and neuraminidase has 9 known subtypes. In humans, currently only about 4 different hemagglutinin and 2 different neuraminidase subtypes are known, e.g., H1, H2, H3, H5, N1, and N2. In particular, two major subtypes of influenza A have been active in humans, namely, H1N1 and H3N2. H1N2, however has recently been of concern. Influenza B viruses are not divided into subtypes based upon their hemagglutinin and neuraminidase proteins.

A reassortant influenza is typically a virus which includes genetic and/or polypeptide components of more than one parental virus strain or source. For example, a 7:1 reassortant influenza virus includes 7 viral genome segments (or gene segments) derived from a first parental virus, and a single complementary viral genome segment, e.g., encoding a hemagglutinin or neuraminidase described herein. A 6:2 reassortant includes 6 genome segments, most commonly the 6 internal genome segments from a first parental virus, and two complementary segments, e.g., hemagglutinin and neuraminidase genome segments, from one or more different parental virus. If the 6:2 reassortant includes 6 viral genome segments derived from a first parental virus, i.e., the 6 internal genome segments, and hemagglutinin and neuraminidase genome segments from more than one different parental virus, it may be referred to as a 6:1:1 reassortant virus. Reassortant viruses can also, depending upon context herein, be termed as “chimeric.”

If the reassortant influenza virus is a recombinant influenza virus it may have been artificially or synthetically (non-naturally) altered by human intervention, e.g., via gene cloning manipulation and reverse genetics. An influenza virus may be recombinant when it is produced by the expression of a recombinant nucleic acid.

The reassortant influenza virus may have a genome segment that encodes a hemagglutinin polypeptide that comprises the amino acid sequence of SEQ ID NO:1. If the genome segment encodes a hemagglutinin polypeptide comprising the amino acid sequence of SEQ ID NO:1 it may have an aspartic acid at amino acid residue position 125, or a glutamic acid residue at amino acid residue position 127, or a glutamic acid at amino acid residue position 209, or an aspartic acid at amino acid residue position 125 and a glutamic acid at amino acid residue position 127, or an aspartic acid at amino acid residue position 125 and a glutamic acid at amino acid residue position 209, or a glutamic acid at amino acid residue position 127 and a glutamic acid at amino acid residue position 209, or an aspartic acid at amino acid residue position 125, a glutamic acid amino acid residue position 127, and a glutamic acid at amino acid residue position 209.

The reassortant influenza virus may have a genome segment that encodes a hemagglutinin polypeptide that comprises the amino acid sequence of SEQ ID NO:3. If the genome segment encodes a hemagglutinin polypeptide comprising the amino sequence of SEQ ID NO:3 it may have a leucine at amino acid residue position 124, or an aspartic acid at amino acid residue position 125, or a glutamic acid at amino acid residue position 127, or a glutamic acid at amino acid residue position 209, or a leucine at amino acid residue position 124 and a glutamic acid at amino acid residue position 209, or an aspartic acid at amino acid residue position 125 and a glutamic acid amino acid residue position 209, or a glutamic acid at amino acid residue position 127 and a glutamic acid at amino acid residue position 209, or a leucine at amino acid residue position 124, a glutamic acid at amino acid residue position 127, and a glutamic acid at amino acid residue position 209, or an aspartic acid at amino acid residue position 125, a glutamic acid at amino acid residue position 127, and a glutamic acid at amino acid residue position 209.

The reassortant influenza virus may have a genome segment that encodes a hemagglutinin polypeptide that comprises the amino acid sequence of SEQ ID NO:4. If the genome segment encodes a hemagglutinin polypeptide comprising the amino acid sequence of SEQ ID NO:4 it may have an aspartic acid at amino acid residue position 125, or a glutamic acid residue at amino acid residue position 127, or a glutamic acid at amino acid residue position 209, or an aspartic acid at amino acid residue position 125 and a glutamic acid at amino acid residue position 127, or an aspartic acid at amino acid residue position 125 and a glutamic acid at amino acid residue position 209, or a glutamic acid at amino acid residue position 127 and a glutamic acid at amino acid residue position 209, or an aspartic acid at amino acid residue position 125, a glutamic acid amino acid residue position 127, and a glutamic acid at amino acid residue position 209.

If the reassortant influenza virus has a genome segment that encodes a hemagglutinin polypeptide comprising the amino acid sequence of SEQ ID NO:1, it may be a 7:1 reassortant influenza virus, a 6:1:1 reassortant influenza virus or a 6:2 reassortant influenza virus. If it is a 6:2 reassortant influenza virus, the reassortant influenza virus may further have a genome segment that encodes a neuraminidase polypeptide comprising the amino acid sequence of SEQ ID NO:5.

If the reassortant influenza virus has a genome segment that encodes a hemagglutinin polypeptide comprising the amino acid sequence of SEQ ID NO:3, it may be a 7:1 reassortant influenza virus, a 6:1:1 reassortant influenza virus or a 6:2 reassortant influenza virus. If it is a 6:2 reassortant influenza virus, the reassortant influenza virus may further have a genome segment that encodes a neuraminidase comprising the amino acid sequence of SEQ ID NO:6. If the reassortant influenza virus has a genome segment that encodes a hemagglutinin polypeptide comprising the amino acid sequence of SEQ ID NO:3 and a genome segment that encodes a neuraminidase polypeptide comprising the amino acid sequence of SEQ ID NO:6, then the amino acid sequence of SEQ ID NO:3 may have a leucine at amino acid residue position 124 and a glutamic acid at amino acid residue position 209, or may have a glutamic acid residue at amino acid residue position 127 and a glutamic acid at amino acid residue position 209, or may have a glutamic acid at amino acid residue position 209.

If the reassortant influenza virus has a genome segment that encodes a hemagglutinin polypeptide comprising the amino acid sequence of SEQ ID NO:4, it may be a 7:1 reassortant influenza virus, a 6:1:1 reassortant influenza virus or a 6:2 reassortant influenza virus. If it is a 6:2 reassortant influenza virus, the reassortant influenza virus may further have a genome segment that encodes a neuraminidase polypeptide comprising the amino acid sequence of SEQ ID NO:8. If the genome segment encodes a neuraminidase polypeptide comprising the amino acid sequence of SEQ ID NO:8, amino acid residue position 222 may be an asparagine, or amino acid residue position 241 may be a valine, or amino acid residue position 369 may be an asparagine, or amino acid residue position 222 may be an asparagine and amino acid residue position 369 may be an asparagine, or amino acid residue position 241 may be a valine and amino acid residue position 369 may be asparagine, or amino acid residue position 222 may be an asparagine, amino acid residue position 241 may be a valine and amino acid residue position 369 may be an asparagine. If the reassortant influenza virus that has a genome segment that encodes a hemagglutinin polypeptide comprising the amino acid sequence of SEQ ID NO:4 is a 6:1:1 reassortant influenza virus it may further have a genome segment that encodes a neuraminidase polypeptide comprising the amino acid sequence of SEQ ID NO:5 or SEQ ID NO:7.

If the reassortant influenza virus is a 6:2 reassortant influenza virus where the genome segment that encodes the hemagglutinin polypeptide comprises the amino acid sequence of SEQ ID NO:4 and the genome segment that encodes the neuraminidase polypeptide comprises the amino acid sequence as shown in SEQ ID NO:8, then the genome segment encoding a hemagglutinin polypeptide may comprise SEQ ID NO:4 wherein amino acid residue position 125 is an aspartic acid and amino acid residue position 127 is a glutamic acid and wherein the genome segment encoding a neuraminidase polypeptide of SEQ ID NO:8 may comprise an asparagine at amino acid position 222, a valine at amino acid residue position 241 and an asparagine at amino acid residue position 369. Alternatively, the genome segment that encodes the hemagglutinin polypeptide may comprise SEQ ID NO:4 where amino acid residue position 125 is an aspartic acid and amino acid residue position 127 is a glutamic acid and the genome segment encoding a neuraminidase polypeptide of SEQ ID NO:8 may comprise an asparagine at amino acid position 369.

In any of the reassortant influenza viruses, e.g., 7:1, 6:2, 6:1:1, the six internal genome segments may be of one any one or more virus, including donor viruses. Donor viruses are generally understood by those of skill in the art. Examples of donor viruses include A/Ann Arbor/6/60 or B/Ann Arbor/1/66, A/Puerto Rico/8/34, B/Leningrad/14/17/55, B/14/5/1, B/USSR/60/69, B/Leningrad/179/86, B/Leningrad/14/55, or B/England/2608/76. If the six internal genome segments are of a single donor virus, the donor virus may be A/Ann Arbor/6/60 or B/Ann Arbor/1/66, A/Puerto Rico/8/34, B/Leningrad/14/17/55, B/14/5/1, B/USSR/60/69, B/Leningrad/179/86, B/Leningrad/14/55, or B/England/2608/76

Any of the reassortant viruses may be in an immunogenic composition. An immunogenic composition may be a composition which is able to enhance an individual's immune response against an antigen, i.e., an influenza virus comprising an hemagglutinin polypeptide comprising all or a portion of the amino acid sequence as shown in SEQ ID NO:1, SEQ ID NO:3, or SEQ ID NO:4 or an influenza virus comprising a neuraminidase polypeptide comprising all or a portion of the amino acid sequence as shown in SEQ ID NO:5, SEQ ID NO:7, or SEQ ID NO:8 Immunogenicity may be monitored, for example, by measuring levels or amounts of neutralizing secretory and/or serum antibodies. An immunogenic composition may be capable of inducing a protective immune response. If the immunogenic composition induces a protective immune response, it may prevent or reduce symptoms caused by infection with wild-type influenza virus comprising an hemagglutinin polypeptide comprising all or a portion of the amino acid sequence as shown in SEQ ID NO:1, SEQ ID NO:3, or SEQ ID NO:4 or an influenza virus comprising a neuraminidase polypeptide comprising all or a portion of the amino acid sequence as shown in SEQ ID NO:5, SEQ ID NO:7, or SEQ ID NO:8.

The reassortant influenza virus in the immunogenic composition may be inactivated. Influenza viruses may be inactivated by use of, for example, formaldehyde and/or b-propiolactone. The reassortant influenza virus in the immunogenic composition may, alternatively, be live attenuated. Such a live attenuated reassortant influenza virus would exhibit such characteristics as, for example, cold adaptation, attenuation, or temperature sensitivity. The terms “temperature sensitive”, “cold adapted” and “attenuated” as applied to viruses are known in the art. For example, the term “temperature sensitive” (ts) indicates, for example, that a virus exhibits a 100 fold or greater reduction in titer at 39° C. relative to 33° C. for influenza A strains, or that the virus exhibits a 100 fold or greater reduction in titer at 37° C. relative to 33° C. for influenza B strains. The term “cold adapted” (ca) indicates that the virus exhibits growth at 25° C. within 100 fold of its growth at 33° C., while the term “attenuated” (att) indicates that the virus replicates in the upper airways of ferrets but is not detectable in their lung tissues, and does not cause influenza-like illness in the animal. It will be understood that viruses with intermediate phenotypes, i.e., viruses exhibiting titer reductions less than 100 fold at 39° C. (for A strain viruses) or 37° C. (for B strain viruses), or exhibiting growth at 25° C. that is more than 100 fold than its growth at 33° C. (e.g., within 200 fold, 500 fold, 1000 fold, 10,000 fold less), and/or exhibit reduced growth in the lungs relative to growth in the upper airways of ferrets (i.e., partially attenuated) and/or reduced influenza like illness in the animal, are also suitable for preparing 6:2 or 6:1:1 or 7:1 reassortant influenza viruses in conjunction with the HA and NA sequences herein.

### Vaccines

An example of an influenza vaccine is FLUMIST (MedImmune, LLC), which is a live, attenuated vaccine that protects children and adults from influenza illness (Belshe et al. 1998 N Engl J Med 338:1405-12; Nichol et al. 1999 JAMA 282:137-44).

FLUMIST vaccine strains contain, for example, HA and NA gene segments derived from the wild-type strains to which the vaccine is addressed (or, in some instances, to related strains) along with six gene segments, PB1, PB2, PA, NP, M and NS, from a common master donor virus (MDV). The HA and NA sequences herein can thus be included in various FLUMIST formulations. The MDV for influenza A strains of FLUMIST (MDV-A), was created by serial passage of the wild-type A/Ann Arbor/6/60 (A/AA/6/60) strain in primary chicken kidney tissue culture at successively lower temperatures (Maassab 1967 Adaptation and growth characteristics of influenza virus at 25 degrees C. Nature 213:612-4). MDV-A replicates efficiently at 25° C. (ca, cold adapted), but its growth is restricted at 38 and 39° C. (ts, temperature sensitive). Additionally, this virus does not replicate in the lungs of infected ferrets (att, attenuation). The ts phenotype is believed to contribute to the attenuation of the vaccine in humans by restricting its replication in all but the coolest regions of the respiratory tract.

Other examples of vaccines include inactivated vaccines FLUZONE® (Sanofi Pasteur), FLUVIRIN (Novartis Vaccines), FLUARIX® (GlaxoSmithKline), FLULAVAL (ID Biomedical Corporation of Quebec), AFLURIA (CSL Biotherapies). These vaccines are produced from influenza viruses containing HA and NA sequences such as those disclosed herein and six internal genome segments of a second, e.g., PR8, influenza virus. Inactivated influenza vaccines may be in split or whole virus form. Typically, inactivated flu vaccines are in a split-virus form.

Vaccines may be formulated to include one or more adjuvants for enhancing the immune response to the influenza antigens. Suitable adjuvants include: complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, bacille Calmette-Guerin (BCG), Corynebacterium parvum, and the synthetic adjuvants QS-21, AS03, and MF59.

Vaccines may also be formulated with or delivered in conjunction with one or more immunostimulatory molecules. Immunostimulatory molecules include various cytokines, lymphokines and chemokines with immunostimulatory, immunopotentiating, and pro-inflammatory activities, such as interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc.

The recombinant and reassortant viruses, immunogenic compositions, and vaccines described herein can be administered prophylactically in an immunologically effective amount and in an appropriate carrier or excipient to stimulate an immune response specific for one or more strains of influenza virus as determined by the HA and/or NA sequence. Typically, the carrier or excipient is a pharmaceutically acceptable carrier or excipient, such as sterile water, aqueous saline solution, aqueous buffered saline solutions, aqueous dextrose solutions, aqueous glycerol solutions, ethanol, allantoic fluid from uninfected hen eggs (i.e., normal allantoic fluid or NAF), or combinations thereof. The preparation of such solutions insuring sterility, pH, isotonicity, and stability is effected according to protocols established in the art. Generally, a carrier or excipient is selected to minimize allergic and other undesirable effects, and to suit the particular route of administration, e.g., subcutaneous, intramuscular, intranasal, etc.

Administration of an immunologically effective amount of recombinant and reassortant virus, immunogenic composition, or vaccine should be in quantities sufficient to stimulate an immune response specific for one or more strains of influenza virus (i.e., against the HA and/or NA influenza antigens described herein). Dosages and methods for eliciting a protective immune response against one or more influenza strains are known to those of skill in the art. See, e.g., U.S. Pat. No. 5,922,326; Wright et al., 1982 Infect. Immun. 37:397-400; Kim et al., 1973 Pediatrics 52:56-63; and Wright et al., 1976 J. Pediatr. 88:931-936. For example, influenza viruses are provided in the range of about 1-1000 HID₅₀ (human infectious dose), i.e., about 10⁵-10⁸ pfu (plaque forming units) per dose administered. Typically, the dose will be adjusted within this range based on, e.g., age, physical condition, body weight, sex, diet, time of administration, and other clinical factors. A vaccine formulation may be systemically administered, e.g., by subcutaneous or intramuscular injection using a needle and syringe, or a needle-less injection device. Alternatively, a vaccine formulation may administered intranasally, either by drops, large particle aerosol (greater than about 10 microns), or spray into the upper respiratory tract. For intranasal administration, attenuated live virus vaccines are often preferred, e.g., an attenuated, cold adapted and/or temperature sensitive recombinant or reassortant influenza virus. See above. While stimulation of a protective immune response with a single dose is preferred, additional dosages can be administered, by the same or different route, to achieve the desired prophylactic effect.

While stimulation of a protective immune response with a single dose is preferred, additional dosages can be administered, by the same or different route, to achieve the desired prophylactic effect. In neonates and infants, for example, multiple administrations may be required to elicit sufficient levels of immunity. Administration can continue at intervals throughout childhood, as necessary to maintain sufficient levels of protection against wild-type influenza infection. Similarly, adults who are particularly susceptible to repeated or serious influenza infection, such as, for example, health care workers, day care workers, family members of young children, the elderly, and individuals with compromised cardiopulmonary function may require multiple immunizations to establish and/or maintain protective immune responses. Levels of induced immunity can be monitored, for example, by measuring amounts of neutralizing secretory and serum antibodies, and dosages adjusted or vaccinations repeated as necessary to elicit and maintain desired levels of protection.

The vaccine may comprise more than one recombinant and/or reassortant influenza virus, i.e., influenza virus(es) in addition to the influenza virus comprising a genome segment encoding a hemagglutinin polypeptide comprising all or a portion of the amino acid sequence of SEQ ID NO:1, SEQ ID NO:3, or SEQ ID NO:4 and/or a neuraminidase polypeptide comprising all or a portion of the amino acid sequence of SEQ ID NO:5, SEQ ID NO:7, or SEQ ID NO:8. The vaccine may be a trivalent vaccine that additionally comprises a recombinant influenza A virus having an H3 HA antigen, and a recombinant influenza B virus having either a Yamagata or Victoria strain HA antigen. The vaccine may be a tetravalent vaccine. If the vaccine is a tetravalent vaccine it may additionally include a recombinant influenza A virus having an HA3 HA antigen, a recombinant influenza B virus having a Yamagata strain HA antigen, and a recombinant influenza B virus having a Victoria strain HA antigen.

### Methods of Making Influenza Virus

Recombinant or reassortant influenza viruses can be readily obtained by a number of methods that are well known in the art. In one method, one or more vectors are introduced into a population of host cells capable of supporting replication of influenza viruses. The one or more vectors comprise nucleotide sequences which correspond to at least six internal genome segments of a first influenza strain and a first genome segment which produces a hemagglutinin polypeptide comprising the amino acid sequence of SEQ ID NO:1 or SEQ ID NO:3, or SEQ ID NO:4.

If the first genome segment produces a hemagglutinin polypeptide comprising the amino acid sequence of SEQ ID NO:1, it may have an aspartic acid at amino acid residue position 125, or a glutamic acid residue at amino acid residue position 127, or a glutamic acid at amino acid residue position 209, or an aspartic acid at amino acid residue position 125 and a glutamic acid at amino acid residue position 127, or an aspartic acid at amino acid residue position 125 and a glutamic acid at amino acid residue position 209, or a glutamic acid at amino acid residue position 127 and a glutamic acid at amino acid residue position 209, or an aspartic acid at amino acid residue position 125, a glutamic acid amino acid residue position 127, and a glutamic acid at amino acid residue position 209. Furthermore, nucleotide sequences corresponding to a second genome segment which produces a neuraminidase polypeptide may also be introduced. The second genome segment may produce a neuraminidase polypeptide comprising the amino acid sequence of SEQ ID NO:5.

If the first genome segment produces a hemagglutinin polypeptide comprising the amino sequence of SEQ ID NO:3 it may have a leucine at amino acid residue position 124, or an aspartic acid at amino acid residue position 125, or a glutamic acid at amino acid residue position 127, or a glutamic acid at amino acid residue position 209, or a leucine at amino acid residue position 124 and a glutamic acid at amino acid residue position 209, or an aspartic acid at amino acid residue position 125 and a glutamic acid amino acid residue position 209, or a glutamic acid at amino acid residue position 127 and a glutamic acid at amino acid residue position 209, or a leucine at amino acid residue position 124, a glutamic acid at amino acid residue position 127, and a glutamic acid at amino acid residue position 209, or an aspartic acid at amino acid residue position 125, a glutamic acid at amino acid residue position 127, and a glutamic acid at amino acid residue position 209. Furthermore, nucleotide sequences corresponding to a second genome segment which produces a neuraminidase polypeptide may be introduced. The second genome segment may produce a neuraminidase polypeptide comprising the amino acid sequence of SEQ ID NO:6.

If the first genome segment produces a hemagglutinin polypeptide comprising the amino acid sequence of SEQ ID NO:4 it may have an aspartic acid at amino acid residue position 125, or a glutamic acid residue at amino acid residue position 127, or a glutamic acid at amino acid residue position 209, or an aspartic acid at amino acid residue position 125 and a glutamic acid at amino acid residue position 127, or an aspartic acid at amino acid residue position 125 and a glutamic acid at amino acid residue position 209, or a glutamic acid at amino acid residue position 127 and a glutamic acid at amino acid residue position 209, or an aspartic acid at amino acid residue position 125, a glutamic acid amino acid residue position 127, and a glutamic acid at amino acid residue position 209. Furthermore, nucleotide sequences corresponding to a second genome segment which produces a neuraminidase polypeptide may be introduced. The second genome segment may produce a neuraminidase polypeptide comprising the amino acid sequence of SEQ ID NO:5, or SEQ ID NO:7, or SEQ ID NO:8. If the second genome segment produces a neuraminidase polypeptide comprising the amino acid sequence of SEQ ID NO:8, amino acid residue position 222 may be an asparagine, or amino acid residue position 241 may be a valine, or amino acid residue position 369 may be an asparagine, or amino acid residue position 222 may be an asparagine and amino acid residue position 369 may be an asparagine, or amino acid residue position 241 may be a valine and amino acid residue position 369 may be asparagine, or amino acid residue position 222 may be an asparagine, amino acid residue position 241 may be a valine and amino acid residue position 369 may be an asparagine.

The nucleotide sequences corresponding to at least 6 internal genome segments of a first influenza strain may be of any influenza strain that provides a useful property for incorporation in an influenza vaccine, or for scientific research, or development purposes. Desirable traits of a first influenza strain may be attenuated pathogenicity or phenotype, cold adaptation, temperature sensitivity. Examples of first influenza strains include A/Ann Arbor/6/60 or B/Ann Arbor/1/66, A/Puerto Rico/8/34, B/Leningrad/14/17/55, B/14/5/1, B/USSR/60/69, B/Leningrad/179/86, B/Leningrad/14/55, or B/England/2608/76.

Vectors for the production of influenza viruses may be, for example, plasmid vectors, which provide one or more origins of replication functional in bacterial and eukaryotic cells, and, optionally, a marker convenient for screening or selecting cells comprising the plasmid sequence. See, e.g., Neumann et al., 1999, PNAS. USA 96:9345-9350.

The vectors may be bi-directional expression vectors capable of initiating transcription of a viral genomic segment from the inserted cDNA in either direction, that is, giving rise to both (+) strand and (−) strand viral RNA molecules. To effect bi-directional transcription, each of the viral genomic segments may be inserted into an expression vector having at least two independent promoters, such that copies of viral genomic RNA are transcribed by a first RNA polymerase promoter (e.g., an RNA pol I promoter), from one strand, and viral mRNAs are synthesized from a second RNA polymerase promoter (e.g., an RNA Pol II promoter). Accordingly, the two promoters can be arranged in opposite orientations flanking at least one cloning site (i.e., a restriction enzyme recognition sequence) preferably a unique cloning site, suitable for insertion of viral genomic RNA segments. Alternatively, an “ambisense” expression vector can be employed in which the (+) strand mRNA and the (−) strand viral RNA (as a cRNA) are transcribed from the same strand of the vector.

The vectors may, alternatively, be unidirectional expression vectors, wherein viral cDNA is inserted between a pol I promoter and a termination sequences (inner transcription unit). This inner transcription unit is flanked by an RNA polymerase II (pol II) promoter and a polyadenylation site (outer transcription unit). In the unidirectional system, the pol I and pol II promoters are upstream of the cDNA and produce positive-sense uncapped cRNA (from the pol I promoter) and positive-sense capped mRNA (from the pol II promoter. See, e.g., Hoffmann and Webster, 2000, J. Gen. Virol. 81:2843-2847.

In other systems, viral sequences transcribed by the pol I and pol II promoters can be transcribed from different expression vectors. In these embodiments, vectors encoding each of the viral genomic segments under the control of a pol I promoter (“vRNA expression vectors”) and vectors encoding one or more viral polypeptides, e.g., influenza PA, PB1, PB2, and NP polypeptides (“protein expression vectors”) under the control of a pol II promoter can be used.

The introduction of the one or more vectors comprising the nucleotide sequences may be by any method or technique known in the art. For example, the vector may be introduced by electroporation, microinjection, and biolistic particle delivery. See, also, e.g., Loeffler and Behr, 1993, Meth. Enzymol. 217:599-618; Cohen et al., 1993, Meth. Enzymol. 217:618-644; Clin. Pharma. Ther. 29:69-92 (1985), Sambrook, et al. Molecular Cloning: A Laboratory Manual. 2nd, ed., Cold Spring Harbor Laboratory, Cold Spring Harbor Laboratory Press, Cold Spring Harbor, N.Y., 1989 and Ausubel et al., ed. Current Protocols in Molecular Biology, John Wiley & Sons, Inc., N.Y., N.Y. (1987-2001).

The introduction of the one or more vectors comprising the nucleotide sequence may also be performed utilizing lipids or liposomes. Lipids or liposomes comprise a mixture of fat particles or lipids which bind to DNA or RNA to provide a hydrophobic coated delivery vehicle. Suitable liposomes may comprise any of the conventional synthetic or natural phospholipid liposome materials including phospholipids from natural sources such as egg, plant or animal sources such as phosphatidylcholine, phosphatidylethanolamine, phosphatidylglycerol, sphingomyelin, phosphatidylserine or phosphatidylinositol. Synthetic phospholipids also may be used, e.g., dimyristoylphosphatidylcholine, dioleoylphosphatidylcholine, dioleoylphosphatidycholine and corresponding synthetic phosphatidylethanolamines and phosphatidylglycerols. Lipids or liposomes that may be conjugated with the vector are also commercially available to the skilled artisan. Examples of commercially available lipid or liposome transfection reagents known to those of skill in the art include LIPOFECTAMINE (Invitrogen), GENEJUICE (Novagen), GENEJAMMER® (Stratagene), FUGENE HD (Roche), MEGAFECTIN (Qbiogene), SUPERFECT (Qiagen), and EFFECTENE (Qiagen).

Furthermore, the introduction of the one or more vectors comprising the nucleotide sequence may be performed by forming compacted polynucleotide complexes or nanospheres. Compacted polynucleotide complexes are described in U.S. Pat. Nos. 5,972,901, 6,008,336, and 6,077,835. Nanospheres are described in U.S. Pat. Nos. 5,718,905 and 6,207,195. These compacted polynucleotide complexes and nanospheres that complex nucleic acids utilize polymeric cations. Typical polymeric cations include gelatin, poly-L-lysine, and chitosan. Alternatively, the polynucleotide of the vector can be complexed with DEAE-dextran, or can be transfected using techniques such as calcium phosphate coprecipitation, or calcium chloride coprecipitation. Introduction of the one or more vectors comprising the nucleotide sequence may or may not result in the nucleotide sequence being incorporated in the chromosome of the host cell.

The population of host cells in which the one or more vectors are introduced are any that are capable of supporting replication of influenza viruses. Many of these host cells are known to those of skill in the art and include MDCK cells, BHK cells, PCK cells, MDBK cells, COS cells, Vero African green monkey kidney cells; the PERC.6 cells (derived from a single human retina-derived cell immortalized using recombinant DNA technology); an EBx stem cell line derived from chicken embryos (Sigma Aldrich). The population of host cells may also refer to combinations or mixtures of cells, for example, a combination of 293 cells (e.g., 293T cells), or COS cells (e.g., COS1, COS7 cells) together with MDCK or VERO or PERC.6 cells.

The population of host cells comprising the one or more vectors is cultured and the influenza virus is recovered. Culturing the host cells can be performed by any of a number of appropriate culture conditions that are known to conducive to influenza virus production. For example, the culturing may be at a temperature less than or equal to 35° C., it may be at between about 32° C. and 35° C., or about 32° C. and about 34° C., or at about 33° C., or at about 32° C., 33° C., 34° C., 35° C., 36° C., 37° C., or 38° C.

Typically, the cultures are maintained in a system, such as a cell culture incubator, under controlled humidity and CO₂, at constant temperature using a temperature regulator, such as a thermostat. The population of cells may be cultured in a standard commercial culture medium, such as Dulbecco's modified Eagle's medium supplemented with serum (e.g., 10% fetal bovine serum), or in serum free medium, under controlled humidity and CO₂ concentration suitable for maintaining neutral buffered pH (e.g., at pH between 7.0 and 7.2). Optionally, the medium contains antibiotics to prevent bacterial growth, e.g., penicillin, streptomycin, etc., and/or additional nutrients, such as L-glutamine, sodium pyruvate, non-essential amino acids, additional supplements to promote favorable growth characteristics, e.g., trypsin, β-mercaptoethanol, and the like. Additional details regarding tissue culture procedures of particular interest in the production of influenza virus in vitro include, e.g., Merten et al. 1996 Production of influenza virus in cell cultures for vaccine preparation. In Cohen and Shafferman (eds) Novel Strategies in Design and Production of Vaccines, which is incorporated herein in its entirety. Additionally, variations in such procedures adapted to the present invention are readily determined through routine experimentation.

Recovering the influenza virus from the cultured population of host cells can be performed by any of a number of ways known and understood by those of skill in the art. For instance, crude medium may be harvested, clarified and concentrated. Common techniques employed by the skilled artisan to recover influenza viruses include filtration, ultrafiltration, adsorption on barium sulfate and elution, and centrifugation. For example, crude medium from cultures can first be clarified by centrifugation at, e.g., 1000-2000×g for a time sufficient to remove cell debris and other large particulate matter, e.g., between 10 and 30 minutes. Alternatively, the medium may be filtered through a 0.8 μm cellulose acetate filter to remove intact cells and other large particulate matter. Optionally, the clarified medium supernatant is then centrifuged to pellet the influenza viruses, e.g., at 15,000×g, for approximately 3-5 hours. Following resuspension of the virus pellet in an appropriate buffer, such as STE (0.01 M Tris-HCl; 0.15 M NaCl; 0.0001 M EDTA) or phosphate buffered saline (PBS) at pH 7.4, the virus is concentrated by density gradient centrifugation on sucrose (60%-12%) or potassium tartrate (50%-10%). Either continuous or step gradients, e.g., a sucrose gradient between 12% and 60% in four 12% steps, are suitable. The gradients may be centrifuged at a speed, and for a time, sufficient for the viruses to concentrate into a visible band for recovery. Alternatively, and for some large scale commercial applications, virus may be elutriated from density gradients using a zonal-centrifuge rotor operating in continuous mode. Additional details sufficient to guide one of skill through the preparation of influenza viruses from tissue culture are provided, e.g., in Furminger. Vaccine Production, in Nicholson et al. (eds) Textbook of Influenza pp. 324-332; Merten et al. (1996) Production of influenza virus in cell cultures for vaccine preparation, in Cohen & Shafferman (eds) Novel Strategies in Design and Production of Vaccines pp. 141-151, and U.S. Pat. No. 5,690,937. If desired, the recovered viruses can be stored at −80° C. in the presence of sucrose-phosphate-glutamate (SPG) as a stabilizer.

### Methods of Increasing the Replication Capacity of Influenza a

The replication capacity of influenza A virus in embryonated eggs may be increased by altering one or more hemagglutinin amino acid residues corresponding to amino acid residue positions 125, 127, and 209 (H1 numbering) to a non-naturally occurring acidic amino acid residue. The alteration may include substituting aspartic acid for the amino acid residue at position 125, or substituting glutamic acid for the amino acid residue at position 127, or substituting glutamic acid for the amino acid residue at position 209, or substituting aspartic acid for the amino acid residue at position 125 and substituting glutamic acid for the amino acid residue at position 127, or substituting aspartic acid for the amino acid residue at position 125 and substituting glutamic acid for the amino acid residue at position 209, or substituting glutamic acid for the amino acid residue at position 127 and substituting glutamic acid for the amino acid residue at position 209, or substituting aspartic acid for the amino acid residue at position 125, substituting glutamic acid for the amino acid residue at position 127, and substituting glutamic acid for the amino acid residue at position 209.

The replication capacity of influenza A virus in embryonated eggs may also be increased by altering one or more neuraminidase amino acid residues corresponding to amino acid residue positions 222, 241, and 369 (N1 numbering) to a non-naturally occurring amino acid residue. The alteration may include substituting asparagine for the amino acid residue at position 222, or substituting valine for the amino acid residue at position 241, or substituting asparagine for the amino acid residue at position 369, or substituting asparagine for the amino acid residue at position 222 and substituting valine for the amino acid residue at position 241, or substituting asparagine for the amino acid residue position 222 and substituting asparagine for the amino acid residue at position 369, or substituting valine for the amino acid residue at position 241 and substituting asparagine for the amino acid residue at position 369, or substituting asparagine for the amino acid residue at position 222 and substituting valine for the amino acid residue at position 241 and substituting asparagine for the amino acid residue at position 369.

The replication capacity of influenza A virus in embryonated eggs may also be increased by altering one or more hemagglutinin amino acid residue corresponding to amino acid residue positions 125, 127, and 209 in combination with one or more neuraminidase amino acid residues corresponding to amino acid residue positions 222, 241, and 369. The alteration may include substituting aspartic acid for the amino acid residue at position 125 in the hemagglutinin polypeptide and substituting asparagine at position 222 in the neuraminidase polypeptide, or substituting aspartic acid for the amino acid residue at position 125 in the hemagglutinin polypeptide and substituting valine at position 241 in the neuraminidase polypeptide, or substituting aspartic acid for the amino acid residue at position 125 in the hemagglutinin polypeptide and substituting asparagine at position 369 in the neuraminidase polypeptide, or substituting aspartic acid for the amino acid residue at position 125 in the hemagglutinin polypeptide and substituting asparagine at position 222 and asparagine at position 369 in the neuraminidase polypeptide, or substituting aspartic acid for the amino acid residue at position 125 in the hemagglutinin polypeptide and substituting asparagine at position 222 and valine at position at 241 in the neuraminidase polypeptide, or substituting aspartic acid for the amino acid residue at position 125 in the hemagglutinin polypeptide and substituting valine at position 241 and an asparagine at position 369 in the neuraminidase polypeptide, or substituting aspartic acid for the amino acid residue at position 125 in the hemagglutinin polypeptide and substituting asparagine at position 222, a valine at position 241 and an asparagine at position 369 in the neuraminidase polypeptide.

The alteration may include substituting glutamic acid for the amino acid residue at position 209 in the hemagglutinin polypeptide and substituting asparagine at position 222 in the neuraminidase polypeptide, or substituting glutamic acid for the amino acid residue at position 209 in the hemagglutinin polypeptide and substituting valine at position 241 in the neuraminidase polypeptide, or substituting glutamic acid for the amino acid residue at position 209 in the hemagglutinin polypeptide and substituting asparagine at position 369 in the neuraminidase polypeptide, or substituting glutamic acid for the amino acid residue at position 209 in the hemagglutinin polypeptide and substituting asparagine at position 222 and asparagine at position 369 in the neuraminidase polypeptide, or substituting glutamic acid for the amino acid residue at position 209 in the hemagglutinin polypeptide and substituting asparagine at position 222 and valine at position 241 in the neuraminidase polypeptide, or substituting glutamic acid for the amino acid residue at position 209 in the hemagglutinin polypeptide and substituting valine at position 241 and an asparagine at position 369 in the neuraminidase polypeptide, or substituting glutamic acid for the amino acid residue at position 209 in the hemagglutinin polypeptide and substituting asparagine at position 222, a valine at position 241 and an asparagine at position 369 in the neuraminidase polypeptide.

The alteration may include substituting glutamic acid for the amino acid residue at position 127 in the hemagglutinin polypeptide and substituting asparagine at position 222 in the neuraminidase polypeptide, or substituting glutamic acid for the amino acid residue at position 127 in the hemagglutinin polypeptide and substituting valine at position 241 in the neuraminidase polypeptide, or substituting glutamic acid for the amino acid residue at position 127 in the hemagglutinin polypeptide and substituting asparagine at position 369 in the neuraminidase polypeptide, or substituting glutamic acid for the amino acid residue at position 127 in the hemagglutinin polypeptide and substituting asparagine at position 222 and asparagine at position 369 in the neuraminidase polypeptide, or substituting glutamic acid for the amino acid residue at position 127 in the hemagglutinin polypeptide and substituting asparagine at position 222 and valine at position 241 in the neuraminidase polypeptide, or substituting glutamic acid for the amino acid residue at position 127 in the hemagglutinin polypeptide and substituting valine at position 241 and an asparagine at position 369 in the neuraminidase polypeptide, or substituting glutamic acid for the amino acid residue at position 127 in the hemagglutinin polypeptide and substituting asparagine at position 222, a valine at position 241 and an asparagine at position 369 in the neuraminidase polypeptide.

The alteration may include substituting aspartic acid for the amino acid residue at position 125 and glutamic acid for the amino acid residue at position 127 in the hemagglutinin polypeptide and substituting asparagine at position 222 in the neuraminidase polypeptide, or substituting aspartic acid for the amino acid residue at position 125 and glutamic acid for the amino acid residue at position 127 in the hemagglutinin polypeptide and substituting valine at position 241 in the neuraminidase polypeptide, or substituting aspartic acid for the amino acid residue at position 125 and glutamic acid for the amino acid residue at position 127 in the hemagglutinin polypeptide and substituting asparagine at position 369 in the neuraminidase polypeptide, or substituting aspartic acid for the amino acid residue at position 125 and glutamic acid for the amino acid residue at position 127 in the hemagglutinin polypeptide and substituting asparagine at position 222 and asparagine at position 369 in the neuraminidase polypeptide, or substituting aspartic acid for the amino acid residue at position 125 and glutamic acid for the amino acid residue at position 127 in the hemagglutinin polypeptide and substituting asparagine at position 222 and valine at position at position 241 in the neuraminidase polypeptide, or substituting aspartic acid for the amino acid residue at position 125 and glutamic acid for the amino acid residue at position 127 in the hemagglutinin polypeptide and substituting valine at position 241 and an asparagine at position 369 in the neuraminidase polypeptide, or substituting aspartic acid for the amino acid residue at position 125 and glutamic acid for the amino acid residue at position 127 in the hemagglutinin polypeptide and substituting asparagine at position 222, a valine at position 241 and an asparagine at position 369 in the neuraminidase polypeptide.

The alteration may include substituting aspartic acid for the amino acid residue at position 125 and glutamic acid for the amino acid residue at position 127 and glutamic acid for the amino acid residue at position 209 in the hemagglutinin polypeptide and substituting asparagine at position 222 in the neuraminidase polypeptide, or substituting aspartic acid for the amino acid residue at position 125 and glutamic acid for the amino acid residue at position 127 and glutamic acid for the amino acid residue at position 209 in the hemagglutinin polypeptide and substituting valine at position 241 in the neuraminidase polypeptide, or substituting aspartic acid for the amino acid residue at position 125 and glutamic acid for the amino acid residue at position 127 and glutamic acid for the amino acid residue at position 209 in the hemagglutinin polypeptide and substituting asparagine at position 369 in the neuraminidase polypeptide, or substituting aspartic acid for the amino acid residue at position 125 and glutamic acid for the amino acid residue at position 127 and glutamic acid for the amino acid residue at position 209 in the hemagglutinin polypeptide and substituting asparagine at position 222 and asparagine at position 369 in the neuraminidase polypeptide, or substituting aspartic acid for the amino acid residue at position 125 and glutamic acid for the amino acid residue at position 127 and glutamic acid for the amino acid residue at position 209 in the hemagglutinin polypeptide and substituting asparagine at position 222 and valine at position at position 241 in the neuraminidase polypeptide, or substituting aspartic acid for the amino acid residue at position 125 and glutamic acid for the amino acid residue at position 127 and glutamic acid for the amino acid residue at position 209 in the hemagglutinin polypeptide and substituting valine at position 241 and an asparagine at position 369 in the neuraminidase polypeptide, or substituting aspartic acid for the amino acid residue at position 125 and glutamic acid for the amino acid residue at position 127 and glutamic acid for the amino acid residue at position 209 in the hemagglutinin polypeptide and substituting asparagine at position 222, a valine at position 241 and an asparagine at position 369 in the neuraminidase polypeptide.

The alteration may include substituting aspartic acid for the amino acid residue at position 125 in the hemagglutinin polypeptide and substituting asparagine for the amino acid residue at position 369 in the neuraminidase polypeptide, or substituting glutamic acid for the amino acid residue at position 127 in the hemagglutinin polypeptide and substituting asparagine for the amino acid residue at position 369 in the neuraminidase polypeptide, or substituting glutamic acid for the amino acid residue at position 209 in the hemagglutinin polypeptide and substituting asparagine for the amino acid residue at position 369 in the neuraminidase polypeptide, or substituting aspartic acid for the amino acid residue at position 125 and substituting glutamic acid for the amino acid residue at position 127 in the hemagglutinin polypeptide and substituting asparagine for the amino acid residue at position 369 in the neuraminidase polypeptide, or substituting aspartic acid for the amino acid residue at position 125 and substituting glutamic acid for the amino acid residue at position 209 in the hemagglutinin polypeptide and substituting asparagine for the amino acid residue at position 369 in the neuraminidase polypeptide, or substituting glutamic acid for the amino acid residue at position 127 and substituting glutamic acid for the amino acid residue at position 209 in the hemagglutinin polypeptide and substituting asparagine for the amino acid residue at position 369 in the neuraminidase polypeptide, or substituting aspartic acid for the amino acid residue at position 125, substituting glutamic acid for the amino acid residue at position 127, and substituting glutamic acid for the amino acid residue at position 209 in the hemagglutinin polypeptide and substituting asparagine for the amino acid residue at position 369 in the neuraminidase polypeptide.

The alteration may include substituting aspartic acid for the amino acid residue at position 125 in the hemagglutinin polypeptide and substituting asparagine for the amino acid residue 222, valine at position 241 and asparagine for the amino acid residue at position 369 in the neuraminidase polypeptide, or substituting glutamic acid for the amino acid residue at position 127 in the hemagglutinin polypeptide and substituting asparagine for the amino acid residue 222, valine at position 241 and asparagine for the amino acid residue at position 369 in the neuraminidase polypeptide, or substituting glutamic acid for the amino acid residue at position 209 in the hemagglutinin polypeptide and substituting asparagine for the amino acid residue 222, valine at position 241 and asparagine for the amino acid residue at position 369 in the neuraminidase polypeptide, or substituting aspartic acid for the amino acid residue at position 125 and substituting glutamic acid for the amino acid residue at position 127 in the hemagglutinin polypeptide and substituting asparagine for the amino acid residue 222, valine at position 241 and asparagine for the amino acid residue at position 369 in the neuraminidase polypeptide, or substituting aspartic acid for the amino acid residue at position 125 and substituting glutamic acid for the amino acid residue at position 209 in the hemagglutinin polypeptide and substituting asparagine for the amino acid residue 222, valine at position 241 and asparagine for the amino acid residue at position 369 in the neuraminidase polypeptide, or substituting glutamic acid for the amino acid residue at position 127 and substituting glutamic acid for the amino acid residue at position 209 in the hemagglutinin polypeptide and substituting asparagine for the amino acid residue 222, valine at position 241 and asparagine for the amino acid residue at position 369 in the neuraminidase polypeptide, or substituting aspartic acid for the amino acid residue at position 125, substituting glutamic acid for the amino acid residue at position 127, and substituting glutamic acid for the amino acid residue at position 209 in the hemagglutinin polypeptide and substituting asparagine for the amino acid residue 222, valine at position 241 and asparagine for the amino acid residue at position 369 in the neuraminidase polypeptide.

The increased replication capacity resulting from the one or more alterations in the hemagglutinin and/or neuraminidase polypeptides results in an influenza virus that grows to a greater titer in embryonated hens' egg relative to a parent influenza virus, e.g., the influenza virus prior to introduction of the one or more alterations in the hemagglutinin and/or neuraminidase polypeptides. The one or more alterations in the hemagglutinin and/or neuraminidase polypeptides may increase the replication capacity by at least about 10%, or by at least about 20%, or by at least about 30%, or by at least about 40%, or by at least about 50%, or by at least about 60%, or by at least about 70%, or by at least about 80%, or by at least about 90%, or by at least about 100%, or by at least about 200%, or by at least about 300%, or by at least about 400%, or by at least about 500% when compared to the parent influenza virus.

The one or more alterations in the hemagglutinin and/or neuraminidase polypeptides may increase the replication capacity of the influenza virus at least 2-fold relative to the parent influenza virus, or may increase the replication capacity at least 4-fold or at least 8-fold, at least 10-fold relative to the parent influenza virus, or at least 100-fold relative to the parent influenza virus.

The one or more alterations in the hemagglutinin and/or neuraminidase polypeptides may increase the replication capacity of the influenza virus to a titer of at least about 7.5 log₁₀ FFU/ml in embryonated eggs, or at least about 8 log₁₀ FFU/ml in embryonated eggs, or at least about 8.1 log₁₀ FFU/ml in embryonated eggs, or at least about 8.2 log₁₀ FFU/ml in embryonated eggs, or at least about 8.3 log₁₀ FFU/ml in embryonated eggs, or at least about 8.4 log₁₀ FFU/ml in embryonated eggs, or at least about 8.5 log₁₀ FFU/ml in embryonated eggs, or at least about 9 log₁₀ FFU/ml in embryonated eggs.

Alterations in the one or more hemagglutinin amino acid residues corresponding to amino acid residue positions 125, 127, and 209 (H1 numbering) and/or one or more neuraminidase amino acid residues corresponding to amino acid residue positions 222, 241, and 369 (N1 numbering) can be made by substituting one or more naturally occurring amino acid residues with an as-herein described non-naturally occurring amino acid residue. The one or more amino acid substitutions may be made by any manipulation technique or set of manipulation techniques well-known to those of skill in the art. Detailed protocols for procedures that may be included in such manipulation(s) may be: amplification, cloning, mutagenesis, transformation, etc., as described in, e.g., in Ausubel et al. Current Protocols in Molecular Biology (supplemented through 2000) John Wiley & Sons, New York (“Ausubel”); Sambrook et al. Molecular Cloning—A Laboratory Manual (2nd Ed.), Vol. 1-3, Cold Spring Harbor Laboratory, Cold Spring Harbor, N.Y., 1989 (“Sambrook”), and Berger and Kimmel Guide to Molecular Cloning Techniques, Methods in Enzymology volume 152 Academic Press, Inc., San Diego, Calif. (“Berger”).

For instance, substitution of selected amino acid residues in viral hemagglutinin and/or neurmimidase polypeptides can be accomplished by, e.g., site-directed mutagenesis. Site-directed mutagenesis may be performed by well-known methods as described, e.g., in Ausubel, Sambrook, and Berger, supra. Numerous kits for performing site directed mutagenesis are also commercially available, e.g., the Chameleon Site Directed Mutagenesis Kit (Stratagene, La Jolla), and can be used according to the manufacturer's instructions to introduce, e.g., one or more amino acid substitutions, into a genome segment encoding an influenza A hemagglutinin and/or neuraminidase polypeptide.

Other manipulation techniques that may be employed to introduce amino acid substitutions in the hemagglutinin and/or neuraminidase polypeptides may include in vitro amplification, such as the polymerase chain reaction (PCR), the ligase chain reaction (LCR), Q-replicase amplification, and other RNA polymerase mediated techniques (e.g., NASBA), are found in Mullis et al. (1987) U.S. Pat. No. 4,683,202; PCR Protocols A Guide to Methods and Applications (Innis et al. eds) Academic Press Inc. San Diego, Calif. (1990) (“Innis”); Arnheim and Levinson (1990) C&EN 36; The Journal Of NIH Research 1991 3:81; Kwoh et al. 1989 Proc Natl Acad Sci USA 86, 1173; Guatelli et al. 1990 Proc Natl Acad Sci USA 87:1874; Lomell et al. 1989 J Clin Chem 35:1826; Landegren et al. 1988 Science 241:1077; Van Brunt 1990 Biotechnology 8:291; Wu and Wallace 1989 Gene 4: 560; Barringer et al. 1990 Gene 89:117, and Sooknanan and Malek 1995 Biotechnology 13:563. Additional methods, useful for cloning nucleic acids, include Wallace et al. U.S. Pat. No. 5,426,039. Improved methods of amplifying large nucleic acids by PCR are summarized in Cheng et al. 1994 Nature 369:684 and the references therein.

Polynucleotides that may be used in the manipulation techniques to introduce amino acid substitutions in the hemagglutinin and/or neuraminidase polypeptide may be, e.g., oligonucleotides that can be synthesized utilizing various solid-phase strategies including mononucleotide- and/or trinucleotide-based phosphoramidite coupling chemistry. For example, nucleic acid sequences can be synthesized by the sequential addition of activated monomers and/or trimers to an elongating polynucleotide chain. See e.g., Caruthers, M. H. et al. 1992 Meth Enzymol 211:3. Oligonucleotides may also be ordered from any of a variety of commercial sources, such as The Midland Certified Reagent Company (Midland, Tex.), The Great American Gene Company (Salt Lake City, Utah), ExpressGen, Inc. (Chicago, Ill.), Operon Technologies, Inc. (Huntsville, Ala.), and many others.

The amino acid positions in the hemagglutinin are based on H1 numbering. The amino acid positions in the neuraminidase are based on N1 numbering. Both hemagglutinin and neuraminidase amino acid numbering schemes are well-known to those of skill in the art. One of skill in the art would readily be able to determine the position of an amino acid residue in any of the H1-H16 influenza A hemagglutinin polypeptides based on the knowledge of the position of the H1 amino acid residue. Likewise, one of skill in the art would readily be able to determine the position of an amino acid residue in any the influenza A N1-N9 neuraminidase polypeptides based on the knowledge of the position of the N1 amino acid residue. The influenza A virus into which the one or more hemagglutinin amino acid residues are altered may be a H1, H2, H3, H5, H6, H7, or H9 influenza A virus.

### Polypeptides

Hemagglutinin polypeptides include all or any portion of the polypeptides as shown in SEQ ID NOs:1, 3, and 4. If the hemagglutinin polypeptide comprises all or a portion of the amino acid sequence as shown in SEQ ID NO:1, it may have an aspartic acid at amino acid residue position 125, or a glutamic acid residue at amino acid residue position 127, or a glutamic acid at amino acid residue position 209, or an aspartic acid at amino acid residue position 125 and a glutamic acid at amino acid residue position 127, or an aspartic acid at amino acid residue position 125 and a glutamic acid at amino acid residue position 209, or a glutamic acid at amino acid residue position 127 and a glutamic acid at amino acid residue position 209, or an aspartic acid at amino acid residue position 125, a glutamic acid amino acid residue position 127, and a glutamic acid at amino acid residue position 209. The hemagglutinin polypeptide may be isolated, or substantially free from components that normally accompany or interact with it in its naturally occurring environment.

If the hemagglutinin polypeptide comprises all or a portion of the amino acid sequence as shown in SEQ ID NO:3, it may have a leucine at amino acid residue position 124, or an aspartic acid at amino acid residue position 125, or a glutamic acid at amino acid residue position 127, or a glutamic acid at amino acid residue position 209, or a leucine at amino acid residue position 124 and a glutamic acid at amino acid residue position 209, or an aspartic acid at amino acid residue position 125 and a glutamic acid amino acid residue position 209, or a glutamic acid at amino acid residue position 127 and a glutamic acid at amino acid residue position 209, or a leucine at amino acid residue position 124, a glutamic acid at amino acid residue position 127, and a glutamic acid at amino acid residue position 209, or an aspartic acid at amino acid residue position 125, a glutamic acid at amino acid residue position 127, and a glutamic acid at amino acid residue position 209. The hemagglutinin polypeptide may be isolated, or substantially free from components that normally accompany or interact with it in its naturally occurring environment.

If the hemagglutinin polypeptide comprises all or a portion of the amino acid sequence as shown in SEQ ID NO:4, it may have an aspartic acid at amino acid residue position 125, or a glutamic acid residue at amino acid residue position 127, or a glutamic acid at amino acid residue position 209, or an aspartic acid at amino acid residue position 125 and a glutamic acid at amino acid residue position 127, or an aspartic acid at amino acid residue position 125 and a glutamic acid at amino acid residue position 209, or a glutamic acid at amino acid residue position 127 and a glutamic acid at amino acid residue position 209, or an aspartic acid at amino acid residue position 125, a glutamic acid amino acid residue position 127, and a glutamic acid at amino acid residue position 209. The hemagglutinin polypeptide may be isolated, or substantially free from components that normally accompany or interact with it in its naturally occurring environment.

Neuraminidase polypeptides include all or any portion of the polypeptides as shown in SEQ ID NOs:5-8. If the neuraminidase polypeptide comprises all or a portion of the amino acid sequence as shown in SEQ ID NO:8 then amino acid residue position 222 may be an asparagine, or amino acid residue position 241 may be a valine, or amino acid residue position 369 may be an asparagine, or amino acid residue position 222 may be an asparagine and amino acid residue position 369 may be an asparagine, or amino acid residue position 241 may be a valine and amino acid residue position 369 may be asparagine, or amino acid residue position 222 may be an asparagine, amino acid residue position 241 may be a valine and amino acid residue position 369 may be an asparagine. The neuraminidase polypeptide may be isolated, or substantially free from components that normally accompany or interact with it in its naturally occurring environment.

The polypeptides may be produced following transduction of a suitable host cell line or strain and growth of the host cells to an appropriate cell density and culturing the cells for an additional period. The expressed polypeptide, e.g., HA and/or NA polypeptide, can then recovered from the culture medium. Alternatively, host cells can be harvested by centrifugation, disrupted by physical or chemical means, and the resulting crude extract retained for further purification. Eukaryotic or microbial cells can be employed in expression of proteins and can then be disrupted by any convenient method, including freeze-thaw cycling, sonication, mechanical disruption, or use of cell lysing agents, or other methods, which are well known to those skilled in the art.

Expressed polypeptides can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography (e.g., using any of the tagging systems known to those skilled in the art), hydroxylapatite chromatography, and lectin chromatography. Protein refolding steps can be used, as desired, in completing configuration of the mature protein. Also, high performance liquid chromatography (HPLC) can be employed in the final purification steps. In addition to the references noted herein, a variety of purification methods are well known in the art, including, e.g., those set forth in Sandana (1997) Bioseparation of Proteins, Academic Press, Inc.; and Bollag et al. (1996) Protein Methods, 2^{nd} Edition Wiley-Liss, NY; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3^{rd} Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ.

The polypeptides may be in a composition alone or in combination with other polypeptides. If polypeptide or polypeptides are in a composition suitable for administration, they may be formulated with physiologically acceptable carriers, excipients, or stabilizers in the form of, e.g., lyophilized powders, slurries, aqueous solutions, lotions, or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.)

If the polypeptides are in combination, the combination may include one, two, three, four, five, six, or more hemagglutinin and/or neuraminidase polypeptides. The composition may comprise a hemagglutinin polypeptide comprising the amino acid sequence of SEQ ID NO:1 with a neuraminidase polypeptide comprising the amino acid sequence of SEQ ID NO:5. The combination may comprise SEQ ID NO:1 having an aspartic acid at amino acid residue position 125 and a neuraminidase polypeptide having the amino acid sequence of SEQ ID NO:5, or may comprise SEQ ID NO:1 having a glutamic acid residue at amino acid residue position 127 and a neuraminidase polypeptide having the amino acid sequence of SEQ ID NO:5, or may comprise SEQ ID NO:1 having a glutamic acid at amino acid residue position 209 and a neuraminidase polypeptide having the amino acid sequence of SEQ ID NO:5, or may comprise SEQ ID NO:1 having an aspartic acid at amino acid residue position 125 and a glutamic acid at amino acid residue position 127 and a neuraminidase polypeptide having the amino acid sequence of SEQ ID NO:5, or may comprise SEQ ID NO:1 having an aspartic acid at amino acid residue position 125 and a glutamic acid at amino acid residue position 209 and a neuraminidase polypeptide having the amino acid sequence of SEQ ID NO:5, or may comprise SEQ ID NO:1 having a glutamic acid at amino acid residue position 127 and a glutamic acid at amino acid residue position 209 and a neuraminidase polypeptide having the amino acid sequence of SEQ ID NO:5, or may comprise SEQ ID NO:1 having an aspartic acid at amino acid residue position 125, a glutamic acid amino acid residue position 127, and a glutamic acid at amino acid residue position 209 and a neuraminidase polypeptide having the amino acid sequence of SEQ ID NO:5.

The composition may comprise a hemagglutinin polypeptide comprising the amino acid sequence of SEQ ID NO:3 and a neuraminidase polypeptide comprising the amino acid sequence of SEQ ID NO:6. The combination may comprise SEQ ID NO:3 having a leucine at amino acid residue position 124 and a neuraminidase polypeptide comprising the amino acid sequence of SEQ ID NO:6, or may comprise SEQ ID NO:3 having an aspartic acid at amino acid residue position 125 and a neuraminidase polypeptide comprising the amino acid sequence of SEQ ID NO:6, or may comprise SEQ ID NO:3 having a glutamic acid at amino acid residue position 127 and a neuraminidase polypeptide comprising the amino acid sequence of SEQ ID NO:6, or may comprise SEQ ID NO:3 having a glutamic acid at amino acid residue position 209 and a neuraminidase polypeptide comprising the amino acid sequence of SEQ ID NO:6, or may comprise SEQ ID NO:3 having a leucine at amino acid residue position 124 and a glutamic acid at amino acid residue position 209 and a neuraminidase polypeptide comprising the amino acid sequence of SEQ ID NO:6, or may comprise SEQ ID NO:3 having an aspartic acid at amino acid residue position 125 and a glutamic acid amino acid residue position 209 and a neuraminidase polypeptide comprising the amino acid sequence of SEQ ID NO:6, or may comprise SEQ ID NO:3 having a glutamic acid at amino acid residue position 127 and a glutamic acid at amino acid residue position 209 and a neuraminidase polypeptide comprising the amino acid sequence of SEQ ID NO:6, or may comprise SEQ ID NO:3 having a leucine at amino acid residue position 124, a glutamic acid at amino acid residue position 127, and a glutamic acid at amino acid residue position 209 and a neuraminidase polypeptide comprising the amino acid sequence of SEQ ID NO:6, or may comprise SEQ ID NO:3 having an aspartic acid at amino acid residue position 125, a glutamic acid at amino acid residue position 127, and a glutamic acid at amino acid residue position 209 and a neuraminidase polypeptide comprising the amino acid sequence of SEQ ID NO:6.

The composition may comprise a hemagglutinin polypeptide comprising the amino acid sequence of SEQ ID NO:4 and a neuraminidase polypeptide comprising the amino acid sequence of SEQ ID NO:5 or SEQ ID NO:6, or SEQ ID NO:8. The composition may comprise SEQ ID NO:4 having an aspartic acid at amino acid residue position 125 and a neuraminidase polypeptide comprising SEQ ID NO:5, or SEQ ID NO:4 having a glutamic acid residue at amino acid residue position 127 and a neuraminidase polypeptide comprising SEQ ID NO:5, or SEQ ID NO:4 having a glutamic acid at amino acid residue position 209 and a neuraminidase polypeptide comprising SEQ ID NO:5, or SEQ ID NO:4 having an aspartic acid at amino acid residue position 125 and a glutamic acid at amino acid residue position 127 and a neuraminidase polypeptide comprising SEQ ID NO:5, or SEQ ID NO:4 having an aspartic acid at amino acid residue position 125 and a glutamic acid at amino acid residue position 209 and a neuraminidase polypeptide comprising SEQ ID NO:5, or SEQ ID NO:4 having a glutamic acid at amino acid residue position 127 and a glutamic acid at amino acid residue position 209 and a neuraminidase polypeptide comprising SEQ ID NO:5, or SEQ ID NO:4 having an aspartic acid at amino acid residue position 125, a glutamic acid amino acid residue position 127, and a glutamic acid at amino acid residue position 209 and a neuraminidase polypeptide comprising SEQ ID NO:5.

The composition may comprise SEQ ID NO:4 having an aspartic acid at amino acid residue position 125 and a neuraminidase polypeptide comprising SEQ ID NO:6, or SEQ ID NO:4 having a glutamic acid residue at amino acid residue position 127 and a neuraminidase polypeptide comprising SEQ ID NO:6, or SEQ ID NO:4 having a glutamic acid at amino acid residue position 209 and a neuraminidase polypeptide comprising SEQ ID NO:6, or SEQ ID NO:4 having an aspartic acid at amino acid residue position 125 and a glutamic acid at amino acid residue position 127 and a neuraminidase polypeptide comprising SEQ ID NO:6, or SEQ ID NO:4 having an aspartic acid at amino acid residue position 125 and a glutamic acid at amino acid residue position 209 and a neuraminidase polypeptide comprising SEQ ID NO:6, or SEQ ID NO:4 having a glutamic acid at amino acid residue position 127 and a glutamic acid at amino acid residue position 209 and a neuraminidase polypeptide comprising SEQ ID NO:6, or SEQ ID NO:4 having an aspartic acid at amino acid residue position 125, a glutamic acid amino acid residue position 127, and a glutamic acid at amino acid residue position 209 and a neuraminidase polypeptide comprising SEQ ID NO:6.

The composition may comprise SEQ ID NO:4 having an aspartic acid at amino acid residue position 125 and a neuraminidase polypeptide comprising SEQ ID NO:8, or SEQ ID NO:4 having a glutamic acid residue at amino acid residue position 127 and a neuraminidase polypeptide comprising SEQ ID NO:8, or SEQ ID NO:4 having a glutamic acid at amino acid residue position 209 and a neuraminidase polypeptide comprising SEQ ID NO:8, or SEQ ID NO:4 having an aspartic acid at amino acid residue position 125 and a glutamic acid at amino acid residue position 127 and a neuraminidase polypeptide comprising SEQ ID NO:8, or SEQ ID NO:4 having an aspartic acid at amino acid residue position 125 and a glutamic acid at amino acid residue position 209 and a neuraminidase polypeptide comprising SEQ ID NO:8, or SEQ ID NO:4 having a glutamic acid at amino acid residue position 127 and a glutamic acid at amino acid residue position 209 and a neuraminidase polypeptide comprising SEQ ID NO:8, or SEQ ID NO:4 having an aspartic acid at amino acid residue position 125, a glutamic acid amino acid residue position 127, and a glutamic acid at amino acid residue position 209 and a neuraminidase polypeptide comprising SEQ ID NO:8. The neuraminidase polypeptide of SEQ ID NO:8 may have an asparagine at position 222, or a valine at position 241, or an asparagine at position 369, or an asparagine at position 222 and an asparagine at position 369, or a valine at position 241 and an asparagine at position 369, or an asparagine at position 222, a valine at position 241 and an asparagine at position 369.

### Polynucleotides

Polynucleotides may encode all or a portion of any of hemagglutinin polypeptides as shown in SEQ ID NOs:1, 3, and 4 or any of neuraminidase polypeptides as shown in SEQ ID NOs:5-8. Examples of polynucleotides include those which comprise all or a part of the sequence as shown in SEQ ID NOs:9-16. Polynucleotides may be DNA, RNA, or other synthetic or modified forms of DNA or RNA molecules. The polynucleotides may be in a vector.

A vector may be the means by which a nucleic acid can be propagated and/or transferred between organisms, cells, or cellular components. Vectors include plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. If the vector is an expression vector it may be capable of promoting expression, as well as replication of a nucleic acid incorporated therein. The vector or expression vector may be incorporated in host cells.

If the vector is an expression vector and the expression vector has been selected for introduction in to bacterial cells, the expression vector may be a multifunctional E. coli cloning and expression vector such as BLUESCRIPT (Stratagene), or a pIN vector (Van Heeke & Schuster 1989 J Biol Chem 264:5503-5509); pET vector (Novagen, Madison Wis.); or any other such well-known expression vectors. Similarly, in the yeast Saccharomyces cerevisiae a number of vectors containing constitutive or inducible promoters such as alpha factor, alcohol oxidase and PGH can be used for production of the desired expression products. For reviews, see Ausubel, infra, and Grant et al., 1987 Methods in Enzymology 153:516-544.

### Host Cells

Host cells may have been transduced, transformed or transfected with vectors, using any number of well-known and commonly practiced techniques. Generally speaking, host cells may be bacterial cells, such as E. coli, Streptomyces, and Salmonella typhimurium; fungal cells, such as Saccharomyces cerevisiae, Pichia pastoris, and Neurospora crassa; insect cells such as Drosophila and Spodoptera frugiperda; or mammalian cells such as COS, Vero, PerC, CHO, BHK, MDCK, 293, 293T, and COST cells.

The host cells comprising a vector or expression vector can be cultured in conventional nutrient media modified as appropriate for activating promoters, selecting transformants, or amplifying the inserted polynucleotide sequences, e.g., through production of viruses. The culture conditions, such as temperature, pH and the like, are typically those previously used with the particular host cell selected for expression, and will be apparent to those skilled in the art and in the references cited herein, including, e.g., Freshney (1994) Culture of Animal Cells, a Manual of Basic Technique, 3^{rd} edition, Wiley-Liss, New York and the references cited therein. Other helpful references include, e.g., Paul (1975) Cell and Tissue Culture, 5^{th} ed., Livingston, Edinburgh; Adams (1980) Laboratory Techniques in Biochemistry and Molecular Biology-Cell Culture for Biochemists, Work and Burdon (eds.) Elsevier, Amsterdam. Additional details regarding tissue culture procedures of particular interest in the production of influenza virus in vitro include, e.g., Merten et al. (1996) Production of influenza virus in cell cultures for vaccine preparation. in Cohen and Shafferman (eds.) Novel Strategies in Design and Production of Vaccines, which is incorporated herein in its entirety for all purposes. Additionally, variations in such procedures adapted to the present invention are readily determined through routine experimentation and will be familiar to those skilled in the art.

### Kits and Reagents

A kit may contain one or more nucleic acid, polypeptide, antibody, or cell line described herein (e.g., comprising, or with, an influenza HA and/or NA molecule comprising all or a portion of any of SEQ ID NOs:1, and 3-8). The kit may contain a diagnostic nucleic acid or polypeptide, e.g., antibody, probe set, e.g., as a cDNA micro-array packaged in a suitable container, or other nucleic acid such as one or more expression vector. The kit may further comprise, one or more additional reagents, e.g., substrates, labels, primers, for labeling expression products, tubes and/or other accessories, reagents for collecting samples, buffers, hybridization chambers, cover slips, etc. The kit optionally further comprises an instruction set or user manual detailing preferred methods of using the kit components for discovery or application of diagnostic sets, etc.

When used according to the instructions, the kit can be used, e.g., for evaluating a disease state or condition, for evaluating effects of a pharmaceutical agent or other treatment intervention on progression of a disease state or condition in a cell or organism, or for use as a vaccine, etc.

Kits may include one or more translation system (e.g., a host cell) with appropriate packaging material, containers, and instructional materials. Furthermore, kits may comprise various vaccines such as live attenuated vaccine (e.g., FluMist) comprising all or a part of any of the HA and/or NA sequences of SEQ ID NOs:1, and 3-8.

### EMBODIMENTS

### Embodiment A1

A recombinant influenza virus comprising a first genome segment encoding a hemagglutinin polypeptide, wherein the hemagglutinin polypeptide comprises the amino acid sequence as shown in: SEQ ID NO:1, SEQ ID NO:3, or SEQ ID NO:4.

### Embodiment A2

The recombinant influenza virus of embodiment A1 wherein the hemagglutinin comprises the amino acid sequence as shown in SEQ ID NO:1.

### Embodiment A3

The recombinant influenza virus of embodiment A2 wherein the hemagglutinin as shown in SEQ ID NO:1 comprises:
an aspartic acid at amino acid residue position 125; or
a glutamic acid at amino acid residue position 127; or
a glutamic acid at amino acid residue position 209; or
an aspartic acid at amino acid residue position 125 and a glutamic acid at amino acid residue position 127; or
an aspartic acid at amino acid residue position 125 and a glutamic acid at amino acid residue position 209; or
a glutamic acid at amino acid residue position 127 and a glutamic acid at amino acid residue position 209; or
an aspartic acid at amino acid residue position 125, a glutamic acid at amino acid residue position 127, and a glutamic acid at amino acid residue position 209.

### Embodiment A4

The recombinant influenza virus of any of embodiments A2 or A3 further comprising a second genome segment encoding a neuraminidase polypeptide, wherein the neuraminidase polypeptide comprises the amino acid sequence as shown in SEQ ID NO:5.

### Embodiment A5

The recombinant influenza virus of embodiment A1 wherein the hemagglutinin comprises the amino acid sequence as shown in SEQ ID NO:3.

### Embodiment A6

The recombinant influenza virus of embodiment A5 wherein the hemagglutinin as shown in SEQ ID NO:3 comprises:
a leucine at amino acid residue position 124; or
an aspartic acid at amino acid residue position 125; or
a glutamic acid at amino acid residue position 127; or
a glutamic acid at amino acid residue position 209; or
a leucine at amino acid residue position 124 and a glutamic acid at amino acid residue position 209; or
an aspartic acid at amino acid residue position 125 and a glutamic acid amino acid residue position 209; or
a glutamic acid at amino acid residue position 127 and a glutamic acid at amino acid residue position 209; or
a leucine at amino acid residue position 124, a glutamic acid at amino acid residue position 127, and a glutamic acid at amino acid residue position 209; or
an aspartic acid at amino acid residue position 125, a glutamic acid at amino acid residue 127, and a glutamic acid at amino acid residue position 209.

### Embodiment A7

The recombinant influenza virus of embodiment A6 wherein the hemagglutinin as shown in SEQ ID NO:3 comprises:
a glutamic acid at amino acid residue position 209; or
a leucine at amino acid residue position 124 and a glutamic acid at amino acid residue position 209; or
a glutamic acid at amino acid residue position 127 and a glutamic acid at amino acid position 209.

### Embodiment A8

The recombinant influenza virus of any of embodiments A5 to A7 further comprising a second genome segment encoding a neuraminidase polypeptide, wherein the neuraminidase polypeptide comprises the amino acid sequence as shown in SEQ ID NO:6.

### Embodiment A9

The recombinant influenza virus of embodiment A1 wherein the hemagglutinin comprises the amino acid sequence as shown in SEQ ID NO:4.

### Embodiment A10

The recombinant influenza virus of embodiment A9 wherein the hemagglutinin as shown in SEQ ID NO:4 comprises:
an aspartic acid at amino acid residue position 125; or
a glutamic acid at amino acid residue position 127; or
a glutamic acid at amino acid residue position 209; or
an aspartic acid at amino acid residue position 125 and a glutamic acid at amino acid position 127; or
a glutamic acid at amino acid residue position 127 and a glutamic acid at amino acid residue position 209; or
an aspartic acid at amino acid residue position 125 and a glutamic acid at amino acid position 209; or
an aspartic acid at amino acid residue position 125, a glutamic acid at amino acid residue position 127, and a glutamic acid at amino acid residue position 209.

### Embodiment A11

The recombinant influenza virus of any of embodiments A9 or A10 further comprising a second genome segment encoding a neuraminidase polypeptide, wherein the neuraminidase polypeptide comprises the amino acid sequence as shown in: SEQ ID NO:5, SEQ ID NO:7, or SEQ ID NO:8.

### Embodiment A12

The recombinant influenza virus of embodiment A11 wherein the neuraminidase polypeptide comprises the amino acid sequence as shown in SEQ ID NO:8.

### Embodiment A13

The recombinant influenza virus of embodiment A12 wherein the neuraminidase polypeptide as shown in SEQ ID NO:8 comprises:
an asparagine at amino acid residue position 222; or
a valine at amino acid residue position 241; or
an asparagine at amino acid residue position 369; or
an asparagine at amino acid residue position 222 and an asparagine at amino acid residue position 369; or
a valine at amino acid residue position 241 and an asparagine at amino acid residue position 369; or
an asparagine at amino acid residue position 222, a valine at amino acid residue 241, and an asparagine at amino acid residue position 369.

### Embodiment A14

The recombinant influenza virus of embodiment A13 wherein the neuraminidase polypeptide as shown in SEQ ID NO:8 comprises:
an asparagine at amino acid residue position 369; or
an asparagine at amino acid residue position 222, valine at amino acid residue 241, and an asparagine at amino acid residue position 369.

### Embodiment A15

The recombinant influenza virus of embodiment A14 wherein:
the neuraminidase polypeptide as shown in SEQ ID NO:8 comprises an asparagine at amino acid residue position 222, valine at amino acid residue 241, and an asparagine at amino acid residue position 369; and
the hemagglutinin polypeptide as shown in SEQ ID NO:4 comprises an aspartic acid at amino acid residue position 125 and a glutamic acid residue at amino acid residue position 127.

### Embodiment A16

The recombinant influenza virus of any of embodiments A1-A15 further comprising six internal genome segments of an influenza virus having phenotypic characteristics of one or more of attenuation, temperature sensitivity, and cold-adaptation.

### Embodiment A17

The recombinant influenza virus of embodiment A16 wherein the six internal genome segments are of influenza virus A/Ann Arbor/6/60.

### Embodiment A18

The recombinant influenza virus of any of embodiments A1-A15 further comprising six internal genome segment of A/Puerto Rico/8/34.

### Embodiment A19

The recombinant influenza virus of any of embodiment A1-A18 which has been inactivated.

### Embodiment A20

The recombinant influenza virus of any of embodiments A1-A17 which is live attenuated.

### Embodiment A21

An immunogenic composition comprising the recombinant influenza virus of embodiment A19 or A20.

### Embodiment A22

A vaccine comprising the immunogenic composition of embodiment A21.

### Embodiment A23

The vaccine of embodiment A22 further comprising at least one other recombinant influenza virus.

### Embodiment A24

The vaccine of embodiment A22 further comprising: a recombinant influenza virus comprising H3N2 influenza A strain HA and NA antigens, a recombinant influenza virus comprising Yamagata influenza B strain HA and NA antigens, and a recombinant influenza virus comprising Victoria influenza B strain HA and NA antigens.

### Embodiment B1

A method of producing the recombinant influenza virus of embodiment A2 comprising:
(a) introducing a plurality of vectors into a population of host cells capable of supporting replication of influenza viruses, which plurality of vectors comprises nucleotide sequences corresponding to at least 6 internal genome segments of a first influenza strain and a first genome segment which produces a hemagglutinin polypeptide comprising the amino acid sequence of SEQ ID NO:1;
(b) culturing the population of host cells; and
(c) recovering the influenza virus.

### Embodiment B2

The method of embodiment B1 wherein the hemagglutinin polypeptide comprising the amino acid sequence of SEQ ID NO:1 comprises:
an aspartic acid at amino acid residue position 125; or
a glutamic acid at amino acid residue position 127; or
a glutamic acid at amino acid residue position 209; or
an aspartic acid at amino acid residue position 125 and a glutamic acid at amino acid residue position 127; or
an aspartic acid at amino acid residue position 125 and a glutamic acid at amino acid residue position 209; or
a glutamic acid at amino acid residue position 127 and a glutamic acid at amino acid residue position 209; or
an aspartic acid at amino acid residue position 125, a glutamic acid at amino acid residue position 127, and a glutamic acid at amino acid residue position 209.

### Embodiment B3

The method of embodiment B1 or B2 comprising, at step (a), further introducing nucleotide sequences corresponding to a second genome segment which produces a neuraminidase polypeptide comprising the amino acid sequence of SEQ ID NO:5.

### Embodiment B4

A method of producing the recombinant influenza virus of embodiment A5 comprising:
(a) introducing a plurality of vectors into a population of host cells capable of supporting replication of influenza viruses, which plurality of vectors comprises nucleotide sequences corresponding to at least 6 internal genome segments of a first influenza strain and a first genome segment which produces a hemagglutinin polypeptide comprising the amino acid sequence of SEQ ID NO:3;
(b) culturing the population of host cells; and
(c) recovering the influenza virus.

### Embodiment B5

The method of embodiment B4 wherein the hemagglutinin polypeptide comprising the amino acid sequence of SEQ ID NO:3 comprises:
a leucine at amino acid residue position 124; or
an aspartic acid at amino acid residue position 125; or
a glutamic acid at amino acid residue position 127; or
a glutamic acid at amino acid residue position 209; or
a leucine at amino acid residue position 124 and a glutamic acid at amino acid residue position 209; or
an aspartic acid at amino acid residue position 125 and a glutamic acid amino acid residue position 209; or
a glutamic acid at amino acid residue position 127 and a glutamic acid at amino acid residue position 209; or
a leucine at amino acid residue position 124, a glutamic acid at amino acid residue position 127, and a glutamic acid at amino acid residue position 209; or
an aspartic acid at amino acid residue position 125, a glutamic acid at amino acid residue 127, and a glutamic acid at amino acid residue position 209.

### Embodiment B6

The method of embodiment B5 wherein the hemagglutinin as shown in SEQ ID NO:3 comprises:
a glutamic acid at amino acid residue position 209; or
a leucine at amino acid residue position 124 and a glutamic acid at amino acid residue position 209; or
a glutamic acid at amino acid residue position 127 and a glutamic acid at amino acid position 209.

### Embodiment B7

The method of any of embodiments B4-B6 comprising, at step (a), further introducing nucleotide sequences corresponding to a second genome segment which produces a neuraminidase polypeptide comprising the amino acid sequence of SEQ ID NO:6.

### Embodiment B8

A method of producing the recombinant influenza virus of embodiment A9 comprising:
(a) introducing a plurality of vectors into a population of host cells capable of supporting replication of influenza viruses, which plurality of vectors comprises nucleotide sequences corresponding to at least 6 internal genome segments of a first influenza strain and a first genome segment which produces a hemagglutinin polypeptide comprising the amino acid sequence of SEQ ID NO:4;
(b) culturing the population of host cells; and
(c) recovering the influenza virus.

### Embodiment B9

The method of embodiment B8 wherein the hemagglutinin polypeptide comprising the amino acid sequence of SEQ ID NO:4 comprises:
an aspartic acid at amino acid residue position 125; or
a glutamic acid at amino acid residue position 127; or
a glutamic acid at amino acid residue position 209; or
an aspartic acid at amino acid residue position 125 and a glutamic acid at amino acid position 127; or
a glutamic acid at amino acid residue position 127 and a glutamic acid at amino acid residue position 209; or
an aspartic acid at amino acid residue position 125 and a glutamic acid at amino acid position 209; or
an aspartic acid at amino acid residue position 125, a glutamic acid at amino acid residue position 127, and a glutamic acid at amino acid residue position 209.

### Embodiment B10

The method of embodiment B8 or B9 comprising, at step (a), further introducing nucleotide sequences corresponding to a second genome segment which produces a neuraminidase polypeptide comprising the amino acid sequence of SEQ ID NO:5, or SEQ ID NO:7, or SEQ ID NO:8.

### Embodiment B11

The method of embodiment B10 wherein the neuraminidase polypeptide comprises the amino acid sequence of SEQ ID NO:8.

### Embodiment B12

The method of embodiment B11 wherein the neuraminidase polypeptide as shown in SEQ ID NO:8 comprises:
an asparagine at amino acid residue position 222; or
a valine at amino acid residue position 241; or
an asparagine at amino acid residue position 369; or
an asparagine at amino acid residue position 222 and an asparagine at amino acid residue position 369; or
a valine at amino acid residue position 241 and an asparagine at amino acid residue position 369; or
an asparagine at amino acid residue position 222, a valine at amino acid residue 241, and an asparagine at amino acid residue position 369.

### Embodiment B13

The method of embodiment B12 wherein the neuraminidase polypeptide as shown in SEQ ID NO:8 comprises:
an asparagine at amino acid residue position 369; or
an asparagine at amino acid residue position 222, valine at amino acid residue 241, and an asparagine at amino acid residue position 369.

### Embodiment B14

The method of embodiment B13 wherein:
the neuraminidase polypeptide as shown in SEQ ID NO:8 comprises an asparagine at amino acid residue position 222, valine at amino acid residue 241, and an asparagine at amino acid residue position 369; and
the hemagglutinin polypeptide as shown in SEQ ID NO:4 comprises an aspatic acid at amino acid residue position 125 and a glutamic acid residue at amino acid residue position 127.

### Embodiment C1

A method of increasing replication capacity of influenza A virus in embryonated eggs comprising:
altering one or more hemagglutinin amino acid residues corresponding to amino acid residue positions 125, 127, and 209 (H1 numbering) to a non-naturally occurring acidic amino acid residue;
whereby the replication capacity of the influenza virus is increased.

### Embodiment C2

The method of embodiment C2 wherein the alteration comprises:
substituting aspartic acid for the amino acid residue at position 125; or
substituting glutamic acid for the amino acid residue at position 127; or
substituting glutamic acid for the amino acid residue at position 209; or
substituting aspartic acid for the amino acid residue at position 125 and substituting glutamic acid for the amino acid residue at position 127; or
substituting aspartic acid for the amino acid residue at position 125 and substituting glutamic acid for the amino acid residue at position 209; or
substituting glutamic acid for the amino acid residue at position 127 and substituting glutamic acid for the amino acid residue at position 209; or
substituting aspartic acid for the amino acid residue at position 125, substituting glutamic acid for the amino acid residue at position 127, and substituting glutamic acid for the amino acid residue at position 209.

### Embodiment C3

The method of embodiment C1 or C2 further comprising altering one or more neuraminidase amino acid residues corresponding to amino acid residue positions 222, 241, or 369 (N1 numbering) to a non-naturally occurring amino acid residue, wherein the alteration comprises:
substituting asparagine for the amino acid residue at position 222; or
substituting valine for the amino acid residue at position 241; or
substituting asparagine for the amino acid residue at position 369; or
substituting asparagine for the amino acid residue at position 222 and substituting valine for the amino acid residue at position 241; or
substituting asparagine for the amino acid residue position 222 and substituting asparagine for the amino acid residue at position 369; or
substituting valine for the amino acid residue at position 241 and substituting asparagine for the amino acid residue at position 369; or
substituting asparagine for the amino acid residue at position 222 and substituting valine for the amino acid residue at position 241 and substituting asparagine for the amino acid residue at position 369.

### Embodiment C4

The method of any of embodiments C1 to C3 wherein the influenza A virus is an influenza H1N1 virus.

### Embodiment C5

An influenza A virus produced by the method of any one of embodiment C1 to C4.

### Embodiment C6

The influenza A virus of embodiment C5 which has been inactivated.

### Embodiment C7

The influenza A virus of embodiment C5 which is live attenuated.

### Embodiment C9

An immunogenic composition comprising the influenza A virus of embodiment C6 or C7.

### Embodiment C10

A vaccine comprising the immunogenic composition according to embodiment C9.

### Embodiment D1

A method of increasing replication capacity of influenza A virus in embryonated eggs comprising:
altering one or more neuraminidase amino acid residues corresponding to amino acid residue positions 222, 241, and 369 (N1 numbering) to a non-naturally occurring amino acid residue, wherein the alteration comprises:
substituting asparagine for the amino acid residue at position 222; or
substituting valine for the amino acid residue at position 241; or
substituting asparagine for the amino acid residue at position 369; or
substituting asparagine for the amino acid residue at position 222 and substituting valine for the amino acid residue at position 241; or
substituting asparagine for the amino acid residue position 222 and substituting asparagine for the amino acid residue at position 369; or
substituting valine for the amino acid residue at position 241 and substituting asparagine for the amino acid residue at position 369; or
substituting asparagine for the amino acid residue at position 222 and substituting valine for the amino acid residue at position 241 and substituting asparagine for the amino acid residue at position 369.
whereby the replication capacity of the influenza virus is increased.

### Embodiment D2

The method of embodiment D1 wherein the influenza A virus is an influenza H1N1 virus.

### Embodiment D3

An influenza A virus produced by the method of any one of embodiment D1 or D2.

### Embodiment D4

The influenza A virus of embodiment D3 which has been inactivated.

### Embodiment D5

The influenza A virus of embodiment D4 which is live attenuated.

### Embodiment D6

An immunogenic composition comprising the influenza A virus of embodiment D4 or D5.

### Embodiment D7

A vaccine comprising the immunogenic composition according to embodiment D6.

### Embodiment E1

An isolated hemagglutinin polypeptide comprising the amino acid sequence as shown in: SEQ ID NO:1, SEQ ID NO:3, or SEQ ID NO:4.

### Embodiment E2

The isolated hemagglutinin polypeptide of embodiment E1 comprising the amino acid sequence as shown in SEQ ID NO:1.

### Embodiment E3

The isolated hemagglutinin polypeptide of embodiment E2 wherein the amino acid sequence as shown in SEQ ID NO:1 comprises:
an aspartic acid at amino acid residue position 125; or
a glutamic acid at amino acid residue position 127; or
a glutamic acid at amino acid residue position 209; or
an aspartic acid at amino acid residue position 125 and a glutamic acid at amino acid residue position 127; or
an aspartic acid at amino acid residue position 125 and a glutamic acid at amino acid residue position 209; or
a glutamic acid at amino acid residue position 127 and a glutamic acid at amino acid residue position 209; or
an aspartic acid at amino acid residue position 125, a glutamic acid at amino acid residue position 127, and a glutamic acid at amino acid residue position 209.

### Embodiment E4

The isolated hemagglutinin polypeptide of embodiment E1 comprising the amino acid sequence of SEQ ID NO:3.

### Embodiment E5

The isolated hemagglutinin polypeptide of embodiment E4 wherein the amino acid sequence as shown in SEQ ID NO:3 comprises:
a leucine at amino acid residue position 124; or
an aspartic acid at amino acid residue position 125; or
a glutamic acid at amino acid residue position 127; or
a glutamic acid at amino acid residue position 209; or
a leucine at amino acid residue position 124 and a glutamic acid at amino acid residue position 209; or
an aspartic acid at amino acid residue position 125 and a glutamic acid amino acid residue position 209; or
a glutamic acid at amino acid residue position 127 and a glutamic acid at amino acid residue position 209; or
a leucine at amino acid residue position 124, a glutamic acid at amino acid residue position 127, and a glutamic acid at amino acid residue position 209; or
an aspartic acid at amino acid residue position 125, a glutamic acid at amino acid residue 127, and a glutamic acid at amino acid residue position 209.

### Embodiment E6

The isolated hemagglutinin polypeptide of embodiment E1 comprising the amino acid sequence of SEQ ID NO:4.

### Embodiment E7

The isolated hemagglutinin polypeptide of embodiment E6 wherein the amino acid sequence of SEQ ID NO:4 comprises:
an aspartic acid at amino acid residue position 125; or
a glutamic acid at amino acid residue position 127; or
a glutamic acid at amino acid residue position 209; or
an aspartic acid at amino acid residue position 125 and a glutamic acid at amino acid position 127; or
a glutamic acid at amino acid residue position 127 and a glutamic acid at amino acid residue position 209; or
an aspartic acid at amino acid residue position 125 and a glutamic acid at amino acid position 209; or
an aspartic acid at amino acid residue position 125, a glutamic acid at amino acid residue position 127, and a glutamic acid at amino acid residue position 209.

### Embodiment E8

An isolated polynucleotide encoding the hemagglutinin polypeptide of any of embodiments E1-E7.

### Embodiment E9

A vector comprising the polynucleotide according to embodiment E8.

### Embodiment E10

A cell comprising the vector according to embodiment E9.

### Embodiment E11

A composition comprising the hemagglutinin polypeptide of any embodiments E1-E7.

### Embodiment E12

The composition of embodiment E11 which is immunogenic.

### Embodiment E13

A composition comprising any of the isolated polynucleotide of embodiment E8, the vector of embodiment E9, or the cell of embodiment E10.

### Embodiment E14

The composition of any of embodiments E11 or E12 further comprising a neuraminidase polypeptide.

### Embodiment E15

The composition of embodiment E14 wherein the neuraminidase polypeptide comprises the amino acid sequence of SEQ ID NO:5, SEQ ID NO:7, or SEQ ID NO:8.

### Embodiment E16

The composition of embodiment E15 wherein the neuraminidase polypeptide comprises the amino acid sequence of SEQ ID NO:8.

### Embodiment E17

The composition of embodiment E16 wherein the neuraminidase polypeptide of SEQ ID NO:8 comprises:
an asparagine at amino acid residue position 222; or
a valine at amino acid residue position 241; or
an asparagine at amino acid residue position 369; or
an asparagine at amino acid residue position 222 and an asparagine at amino acid residue position 369; or
a valine at amino acid residue position 241 and an asparagine at amino acid residue position 369; or
an asparagine at amino acid residue position 222, a valine at amino acid residue 241, and an asparagine at amino acid residue position 369.

### Embodiment F1

An isolated neuraminidase polypeptide comprising the amino acid sequence of SEQ ID NO:5, SEQ ID NO:7, or SEQ ID NO:8.

### Embodiment F2

The isolated neuraminidase polypeptide according to embodiment F1 which comprises the amino acid sequence as shown in SEQ ID NO:8.

### Embodiment F3

The isolated neuraminidase polypeptide according to embodiment F2 wherein the neuraminidase polypeptide of SEQ ID NO:8 comprises:
an asparagine at amino acid residue position 222; or
a valine at amino acid residue position 241; or
an asparagine at amino acid residue position 369; or
an asparagine at amino acid residue position 222 and an asparagine at amino acid residue position 369; or
a valine at amino acid residue position 241 and an asparagine at amino acid residue position 369; or
an asparagine at amino acid residue position 222, a valine at amino acid residue 241, and an asparagine at amino acid residue position 369.

### Embodiment F4

An isolated polynucleotide encoding the neuraminidase polypeptide of any of embodiments F1-F3.

### Embodiment F5

A vector comprising the polynucleotide according to embodiment F4.

### Embodiment F6

A cell comprising the vector according to embodiment F5.

### Embodiment F7

A composition comprising the neuraminidase polypeptide of any of embodiments F1-F3, the polynucleotide of embodiment F4, the vector of embodiment F5 or the cell of embodiment F6.

### EXAMPLES

The invention is now described with reference to the following examples. These examples are provided for the purpose of illustration only and the invention should in no way be construed as being limited to these examples but rather should be construed to encompass any and all variations which become evident as a result of the teachings provided herein.

### 1. Materials and Methods

Wild Type Viruses:

Egg-grown wild type H1N1pdm viruses A/Brisbane/10/2010 and A/New Hampshire/2/2010 were kindly provided by the Centers for Disease Control and Prevention, USA. A/Gilroy/231/2011 was isolated from the nasal wash of a ferret which contracted human influenza transmitted from a husbandry staff. All the viruses were expanded in both Madin Darby canine kidney (MDCK) cells (European Collection of Cell Cultures) and embryonated chicken eggs (Charles River Laboratories, Wilmington, Mass.).

Generation of Recombinant Viruses by Reverse Genetics:

The HA and NA gene segments of wt H1N1pdm viruses were amplified by RT-PCR and cloned into the pAD3000 vector (Hoffmann et al., 2000 Proc Natl Acad Sci USA. 97:6108-13). Site-directed mutagenesis was performed to introduce specific changes into the HA and NA genes using the QuikChange® Site-Directed Mutagenesis kit (Agilent Technologies, Santa Clara, Calif.). The 6:2 reassortant vaccine viruses were generated by plasmid rescue as described previously (Jin et al., 2003 Virology 306:18-24). Briefly, the 6:2 reassortant candidate vaccine viruses were generated by co-transfecting eight cDNA plasmids encoding the HA and NA protein gene segments of the H1N1 virus and the six internal protein gene segments of cold-adapted A/Ann Arbor/6/60 (AA ca, H2N2) virus into co-cultured 293T and MDCK cells. The rescued viruses from the cell supernatants were propagated in the allantoic cavity of 10- to 11-day-old embryonated chicken eggs. The HA and NA sequences of the viruses were verified by sequencing RT-PCR cDNAs amplified from vRNA.

Virus Titration:

Infectious virus titers were measured by the fluorescence focus assay (FFA) in MDCK cells and expressed as log₁₀FFU (fluorescent focus units)/ml. Virus plaque morphology was examined by plaque assay as described before (Lu et al., 2005 J. Virol. 79:6763-6771). To compare the replication of 6:2 reassortant viruses in eggs, eggs were inoculated with 10³FFU/egg of virus and incubated at 33° C. for 3 days. Allantoic fluid was harvested for both FFA assay and plaque assay.

Virus Growth Kinetics and Virus Protein Expression:

The growth kinetics of recombinant 6:2 reassortants were determined in MDCK cells. MDCK cells were inoculated with the viruses at a multiplicity of infection (MOI) of 5 or 0.005. After 1 hr of adsorption, the infected cells were washed with PBS and incubated with minimal essential medium (MEM) containing 1 g/ml TPCK-trypsin (Sigma-Aldrich, St. Louis, Mo.) and incubated at 33° C. The cell culture supernatant was collected at different time points and the virus titer was determined by FFA assay.

Viral proteins produced in the infected cells and released virions in cell culture supernatants were analyzed by western blot. MDCK cells were infected with the viruses at an MOI of 5 as described above. At 8 hr and 16 hr post-infection, the cell culture supernatant was collected and cellular debris was removed by centrifugation in microcentrifuge at 14,000 rpm for 5 min. The infected cells were collected and lysed with RIPA buffer (20 mM TrisCl [pH7.5], 150 mM NaCl, 1% Triton X-100, 0.5% sodium deoxycholate, 0.1% SDS, protease inhibitor cocktail). Equal amount of cell lysate and cell supernatant were electrophoresed on a Novex® 12% Tris-Glycine gel (Invitrogen, Carlsbad, Calif.) under the denaturing condition. The proteins were transferred to a nitrocellulose membrane and blotted with influenza specific antibodies.

For immunofluorescence assay, MDCK cells were infected with the viruses at an MOI of 0.005. At 15 hrs or 48 hrs of post-infection, infected cells were fixed with 10% formalin for 20 minutes followed by treatment with ice cold methanol for 5 minutes. The cells were then incubated with goat anti-influenza A virus polyclonal antibody (Millipore, Bedford, Mass.) at a dilution of 1:40 at room temperature for 1 hr, followed by incubation with FITC-conjugated rabbit anti-goat IgG antibody (Millipore, Bedford, Mass.) at a dilution of 1:100 for 30 min. The stained cells were examined by a fluorescence microscope.

Serum Antibody Detection by HAI Assay:

Eight to ten week-old male and female ferrets (n=3/group) from Simonsen Laboratories (Gilroy, Calif.) were inoculated intranasally with 7.0 log₁₀FFU of virus per 0.2 ml dose. Ferret serum samples were collected 14 days after infection. HAI assay was used to determine antibody levels in post-infected ferret sera against homologous and heterologous viruses. 25 μl of serial-diluted serum samples treated with receptor-destroying enzyme (RDE, Denka Seiken Co., Tokyo, Japan) were mixed with 4 HA units of the indicated viruses (25 μl) in 96-well V-bottom microplates. After incubating at room temperature for 30 min, 50 μl of 0.5% chicken erythrocytes (cRBC) were added to each well and incubated for an additional 45 min. The HAI titer was defined as the reciprocal of the highest serum dilution that inhibited virus hemagglutination.

### 2. H1N1pdm Vaccine Strains Grew Differently in Embryonated Chicken Eggs

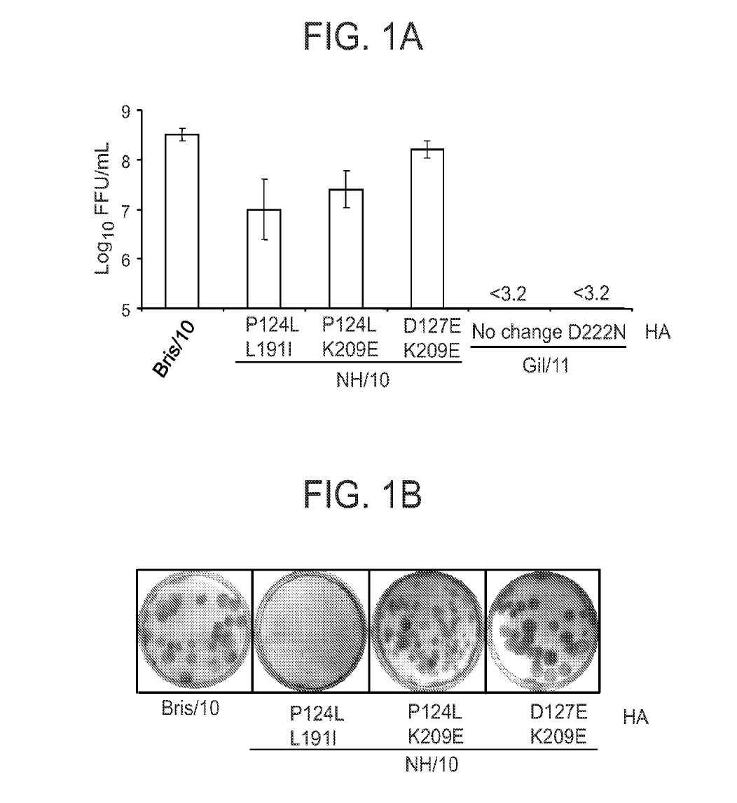

Three recent H1N1pdm strains, A/Brisbane/10/2010 (Bris/10), A/New Hampshire/2/2010 (NH/10) and A/Gilroy/231/2011 (Gil/11), exhibited sequence variations in both HA and NA gene segments compared to A/California/7/2009 (CA/09). Egg adaptation sequence changes were observed at multiple HA positions such as 119, 124, 127, 191, 209 and 222 (Table 1, below). To evaluate the growth of LAIV vaccine candidates of these viruses, the 6:2 cold-adapted (ca) reassortant viruses containing the 6 internal protein gene segments from the master donor virus A/Ann Arbor/6/60 ca and the HA and NA genes from the wild type (wt) H1N1pdm viruses were generated using the eight-plasmid reverse genetics system. The rescued viruses were amplified in embryonated chicken eggs and infectious virus titers were determined by the fluorescence focus assay (FFA) assay. Plaque morphology was examined by plaque assay in MDCK cells (FIG. 1). The HA gene of egg derived Bris/10 wt was homogeneous (Table 1). In contrast to CA/09, Bris/10 ca grew efficiently to a titer of 8.5 log₁₀FFU/ml and formed big plaques in MDCK cells. Three HA variants with the egg adaptation changes in the HA (P124L/L191I, P124L/K209E and D127E/K209E) were cloned from NH/10 wt virus. The respective NH/10 ca variants grew to different titers in eggs and had distinct plaque morphology. The P124L/L191I variant had low titer in eggs and formed tiny plaques in MDCK cells. Both P124L/K209E and D127E/K209E ca viruses formed big plaques and the D127E/K209E variant reached the titer of 8.2 log₁₀FFU/ml, indicating that the K209E change mainly contributed to the efficient virus growth. The Gil/11 6:2 ca viruses containing the original HA sequence or the HA with an egg adaptation change (D222N) could not be recovered from the plasmids transfected cells. Correspondingly, Bris/10 wt and the NH/10 wt isolate containing D127E/K209E grew efficiently, while Gil/11 wt grew poorly in both MDCK cells and eggs (data not shown), indicating that the HA and NA genes controlled virus replication. Sequence comparison of these high and low growth viruses indicated that the HA residues at positions 125, 127 and 209 may be important for virus growth in eggs.

TABLE 1
The HA sequence comparison of recent H1N1pdm strains

| | Hemagglutinin (H1 numbering) | | | | | | | | | | | | | | | |
|---|---|---|---|---|---|---|---|---|---|---|---|---|---|---|---|---|
| | 83 | 97 | 124 | 125 | 127 | 191 | 203 | 205 | 209 | 216 | 222 | 249 | 283 | 300 | 321 | 374 |
| A/California/7/2009 | P | D | P | N | D | L | S | R | K | I | D | V | K | I | I | E |
| A/Brisbane/10/2010 | S | | | D | E | | T | | | | | | | | V | K |
| A/New | S | N | /L | | /E | /I | T | | /E | | | | | | V | K |
| Hampshire/2/2010 | | | | | | | | | | | | | | | | |
| A/Gilroy/231/2011 | S | N | | | | /I | T | K | | V | /N | L | E | L | V | K |

/X: mixed sequences; X: egg adaptation changes
The HA sequences of the egg-adapted H1N1pdm viruses A/Brisbane/10/2010, A/New Hampshire/2/2010 and A/Gilroy/231/2011 were compared with the wild-type A/Califomia/07/09 reference strain. Only the residues that different from A/California/07/09 are listed.

3. Identification of the HA Residues that Support High Growth of Vaccine Viruses

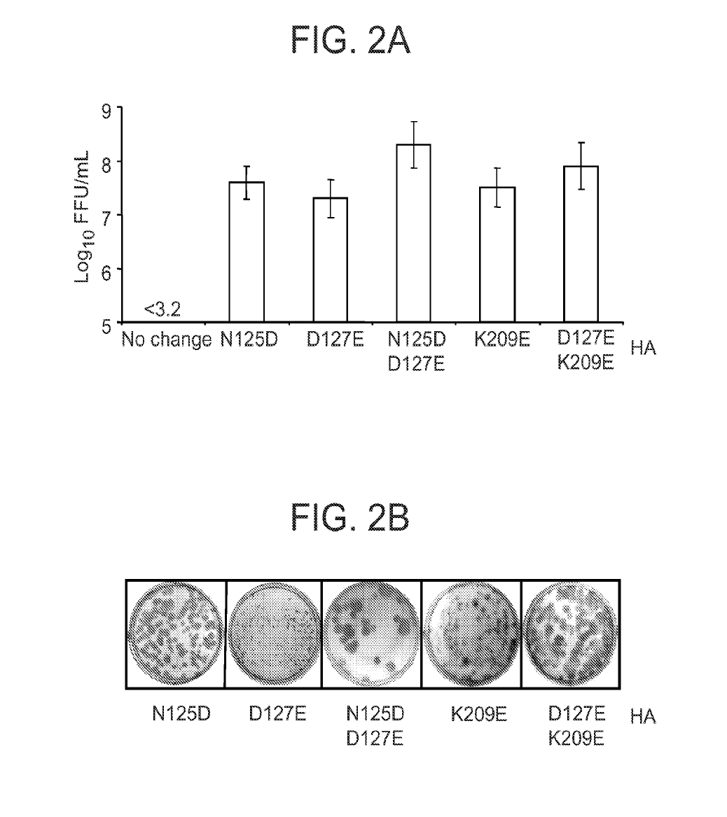

Recombinant CA/09 ca viruses containing the original HA sequence had prior been shown to not be recovered from plasmid DNA transfected cells. The HA D222G change in the receptor binding domain enabled virus recovery but the virus titer was low. The K119E and A186D substitutions in the HA greatly improved virus growth, reaching a titer of approximately 8.5 log₁₀ FFU/ml (Chen et al., 2010 J. Virol. 84:44-51). To confirm that the newly identified amino acid substitutions (N125D, D127E and K209E) conferred a growth advantage of H1N1pdm vaccine viruses in eggs, each of the identified mutations was introduced into the cDNA of the original CA/09 HA individually or in combination. The 6:2 ca reassortant viruses were rescued and examined for their growth in eggs (FIG. 2). All the single mutations (N125D, D127E or K209E) significantly improved virus growth in eggs. In addition, the virus with N125D formed big plaques in MDCK cells. The double mutations further improved virus replication, reaching the highest titer at approximately 8.3 log₁₀ FFU/ml, which was comparable to Bris/10 in virus titer and plaque size (FIG. 1). Thus, in addition to the K119E and A186D substitutions we identified previously, the N125D, D127E and K209E change in the HA also greatly facilitated vaccine virus growth.

4. Both the HA and NA were Required for High Growth of Gil/11

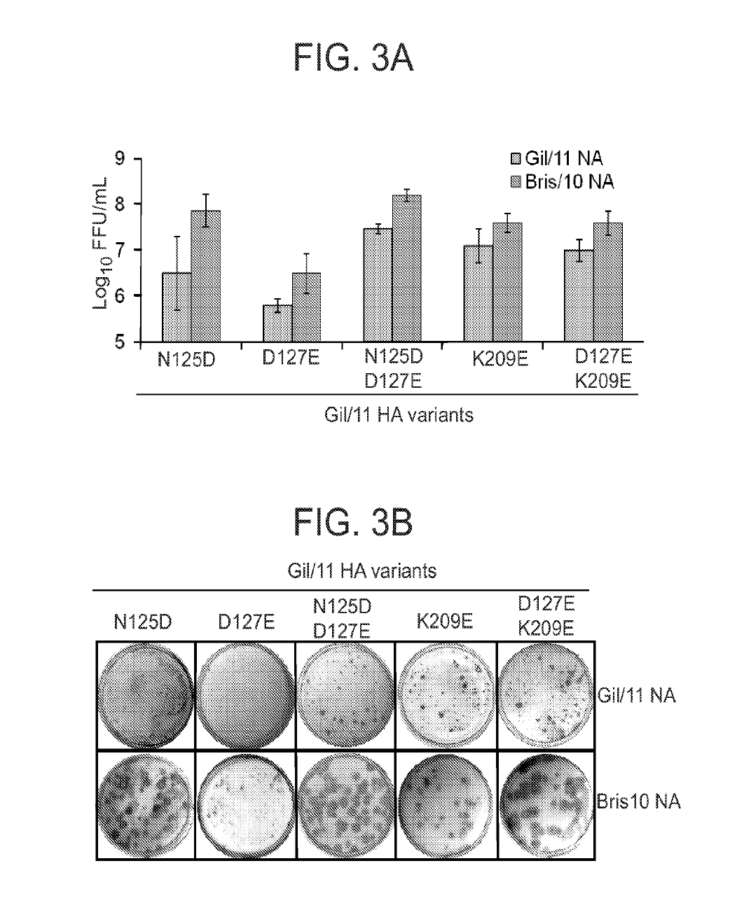

To determine whether the substitutions at HA positions of 125, 127 and 209 could also improve Gil/11 ca virus growth in eggs, single or double HA mutations were introduced into the Gil/11 ca virus (FIG. 3A, left columns). Although all the HA variants were rescued, they all formed tiny or small plaques (FIG. 3B upper panel) with low infectious titers of 6.5 to 7.7 log₁₀ FFU/ml. These data suggested that the changes in the HA could not completely improve the growth of Gil/11 ca virus.

To assess the possible contribution of the NA protein to virus growth, the NA segment of these Gil/11 ca HA variants was replaced with Bris/10 NA by reverse genetics and the recovered Gil/11 ca HA variants with the NA segment from Bris/10 were examined for their growth in eggs. As shown in FIG. 3, all the viruses with Bris/10 NA grew to higher titers than the corresponding viruses with Gil/11 NA. The replacement of Gil/11 NA with CA/09 NA similarly improved virus growth (data not shown). The Gil/11 ca variant containing the N125D/D127E double mutation in the HA and the Bris/10 NA grew to a highest titer in eggs (8.2 log₁₀ FFU/ml) and formed large plaques. These data demonstrated that both HA and NA proteins contribute to virus replication in eggs and MDCK cells and implied that the HA receptor binding and NA receptor cleaving function of the Gil/11 virus were not well balanced from virus replication in host cells.

It is worth noting that no significant difference in Gil/11 and Bris/10 NA enzymatic activity was detected using a MUN substrate (data not shown).

5. Identification of the NA Residues that Contribute to Efficient Growth of Gil/11 Ca Virus in Eggs

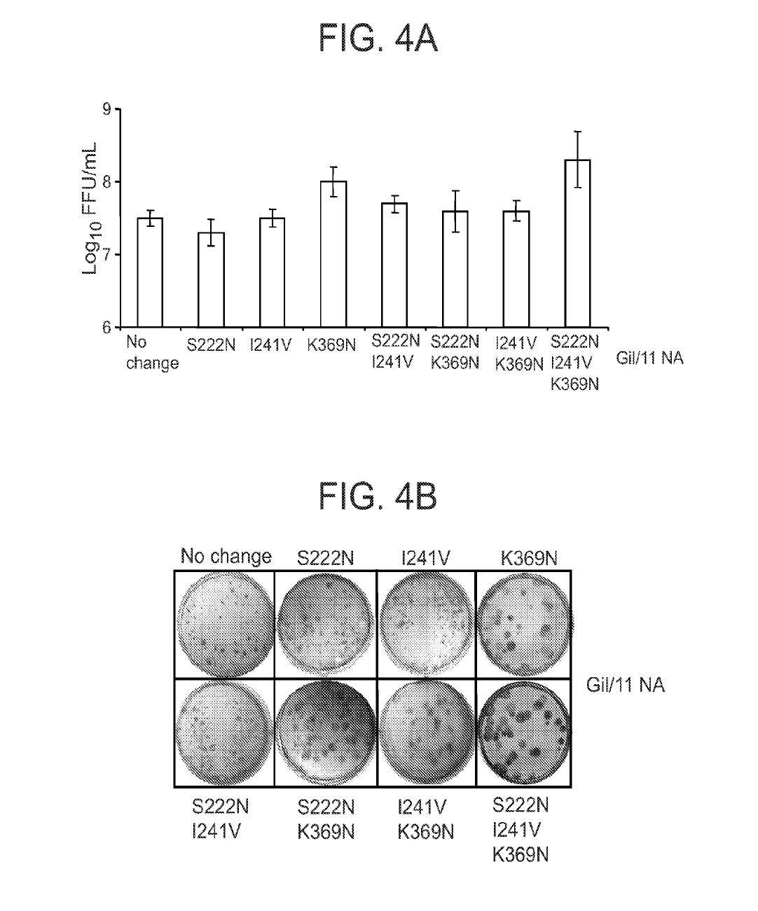

Sequence comparison showed that Gil/11 had five unique NA residues at positions 44, 222, 241, 369 and 443 (N1 numbering) compared with the NA of CA/09 and Bris/10 (Table 2). To identify if any of these NA amino acid substitutions were responsible for the lower growth of Gil/11 ca, 544N, S222N, I241V, K369N and M443I changes were introduced into the Gil/11 (N125D/D127E in HA) ca virus. As shown in FIG. 4, the three single mutations of S222N, I241V and K369N (corresponding N2 numbering 221, 241, and 369, respectively) improved virus growth in eggs. The K369N change was most important, which increased virus titer by 0.5 log₁₀FFU/ml and improved virus plaque size. The S44N and M443I changes did not affect virus growth (data not shown). The double mutations had no additional effect on viral growth compared with the single mutations. However, a triple NA mutant with changes at NA residues 222, 241 and 369 had the highest virus titer of 8.3 log₁₀ FFU/ml and large plaque morphology, comparable to the virus with Brisbane NA. Thus, not only the HA protein but also the NA accounted for the poor growth of Gil/11 ca.

TABLE 2
The NA sequence comparison of recent H1N1pdm strains

| | Neuraminidase (N1 numbering) | | | | | | | | | | |
|---|---|---|---|---|---|---|---|---|---|---|---|
| | 11 | 15 | 44 | 106 | 189 | 222 | 241 | 248 | 369 | 419 | 443 |
| A/California/7/2009 | G | M | N | V | N | N | V | N | N | R | I |
| A/Brisbane/10/2010 | | I | | I | S | | | D | | K | |
| A/New | S | | | I | | | | D | | | |
| Hampshire/2/2010 | | | | | | | | | | | |
| A/Gilroy/231/2011 | | | S | I | | S | I | D | K | | M |

/X: mixed sequences; X: egg adaptation changes
The NA sequences of the egg-adapted H1N1pdm viruses A/Brisbane/10/2010, A/New Hampshire/2/2010 and A/Gilroy/231/2011 were compared with the wild-type A/Califomia/07/09 reference strain. Only the residues that different from A/California/07/09 are listed.

### 6. The Effect of the HA Residues on Virus Immunogenicity and Antigenicity

To assess whether the HA changes in these high-growth ca variants affect virus antigenicity and immunogenicity, the Bris/10, NH/10 with D127E/K209E in HA (NH/10 v1) and Gil/11 with N125D/D127E in HA and Bris/10 NA (Gil/11 v1) ca viruses were examined for their immunogenicity and antigenicity in ferrets. Ferrets were inoculated intranasally with 7.0 log₁₀ FFU of the above vaccine candidates and ferret serum was collected on day 14. The antibody titers against homologous and heterologous H1N1pdm viruses were evaluated by HAI assay (Table 3). All the Bris/10, NH/10 and Gil/11 ca viruses were immunogenic and induced high HAI antibody titers (912-2048) against homologous viruses. Similar to current CA/09 LAIV, they all cross-reacted well to the H1N1pdm wt viruses and the heterologous viruses (HAI titers were within 4-fold compared to homologous titers). For example, viruses containing HA N125D/D127E (Bris/10 and Gil/11) immunized ferret sera cross-reacted well to viruses containing HA D127E/K209E (NH/10 v1 and Gil/11 v2) or HA P124L/L191I (NH/10 v2). Thus, the N125D/D127E or D127E/K209E substitutions in the HA of both Gil/11 and NH/10 did not alter virus antigenicity and these newer strains, Bris/10, NH/10 and Gil/11, can serve as H1N1pdm vaccines.

TABLE 3
The immunogenicity and antigenicity of vaccine variants in ferrets

| | | | | | | GMT HAI titers of ferret serum immunized | | | |
|---|---|---|---|---|---|---|---|---|---|
| | | | | | | with ca viruses | | | |
| | HA residues | | | | | CA/09 | | | |
| Test viruses | 124 | 125 | 127 | 191 | 209 | LAIV¹ | Bris/10 | NH/10 v1 | Gil/11 v1 |
| CA/09 LAIV | P | N | D | L | K | 861 | 724 | 1448 | 724 |
| CA/09 wt | | | | L/I | | 1024 | 1024 | 1448 | 2896 |
| Bris/10 | | D | E | | | 470 | 912 | 1024 | 724 |
| NH/10 v1 | | | E | | E | 790 | 813 | 2048 | 512 |
| NH/10 v2 | L | | | I | | 362 | 575 | 1024 | 724 |
| NH/10 wt | L/I | | D/E | L/I | K/E | 724 | 1149 | 1024 | 724 |
| Gil/11 v1 | | D | E | | | 472 | 2048 | 2048 | 2048 |
| Gil/11 v2 | | | E | | E | 1024 | 512 | 2048 | 1448 |
| Gil/11 wt | | | | | | 470 | 406 | 1024 | 724 |

Groups of ferrets were inoculated intranasally with 10^{7.0}FFU of the indicated H1N1pdm ca vaccine viruses. Serum was collected 14 days after immunization and the antibody titers against different teste were determined by the hemagglutination inhibition assay (HAI) using chicken erythrocytes. The HA sequence variations at the positions of 124, 125, 127, 191 and 209 of the test viruses are indicated. The HAI titers against homologous viruses were underlined.
¹The current LAIV vaccine strain contain the changes at the other sites of HA (119, 186 and 222) that improved vaccine virus growth.

7. The HA and NA Substitutions Improve Virus Growth by Facilitating Virus Release from Infected Cells

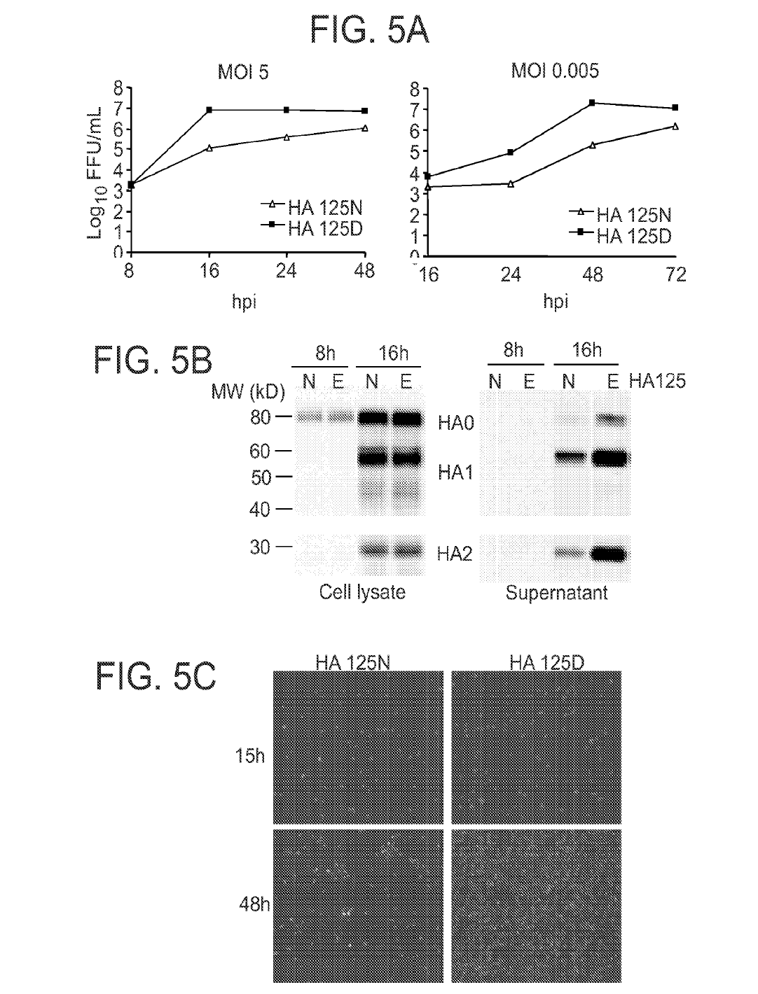

The identified HA amino acids that improved vaccine virus growth all contained acidic amino acid substitutions (K119E, A186D, N125D, and K209E). To investigate the impact of these residues on virus replication, pairs of viruses with or without the acidic residue changes were compared for their growth kinetics in MDCK cells. The representative data of the low-growth virus CA09-D127E (125N) vs. the high-growth virus CA/09-N125D/D127E (125D) are shown in FIG. 5A. The 125N virus showed lower replication kinetics than the 125D virus at both high MOI and low MOI, indicating that the multi-cycle replication of the 125N virus was impaired. The peak titers of the 125D virus at MOI 5 (16 hpi) or MOI 0.005 (48 hpi) were approximately 2 logs higher than the 125N virus.

Viral protein levels in the infected cells and the culture supernatants at different time points with a high MOI were examined by western blot (FIG. 5B). The 125N and 125D viruses produced comparable amounts of viral proteins in the infected cells from 8 to 16 hr postinfection. However, the amount of viral particles released into the supernatants of cells infected with the 125N virus, as detected by viral protein levels, was much lower than the high-growth 125D virus. The data indicated that the low-growth viruses could enter cells and initiate RNA transcription and protein synthesis efficiently, but virus release from infected cells was not efficient resulting in poor virus spread or multi-cycle replication. These were reflected in small virus plaques in MDCK cells and lower titers in eggs and MDCK cells.

The difference of the two viruses in virus spread at a low MOI was confirmed by immunofluorescence (FIG. 5C). At MOI of 0.005, a similar percentage of cells was infected at 15 hr postinfection for both viruses. At 48 hr postinfection, the majority of the cells were infected by the 125D virus; however, only a low percentage of cells was infected with the 125N virus. Similar results were obtained with other pairs of viruses such as CA/09 (D222G) vs. CA/09 (D222G)-K119E/A186D, indicating that the acidic residue changes in the HA facilitated virus release from cells.

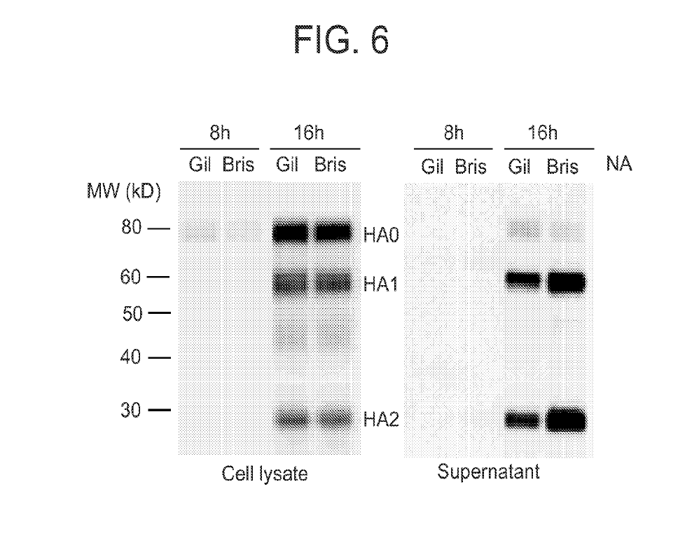

To investigate the effect of NA on virus replication in MDCK cells, Gil/11 ca viruses containing Gil/11 NA or Bris/10 NA were also compared for the viral protein expression in the infected cells (FIG. 6). Similarly, the two viruses showed similar protein expression in infected cells, but the virus with Gil/11 NA had inefficient virus spread compared to the virus containing Bris/10 NA.

8. Structural Context of the HA and NA Substitutions that Improve Virus Growth

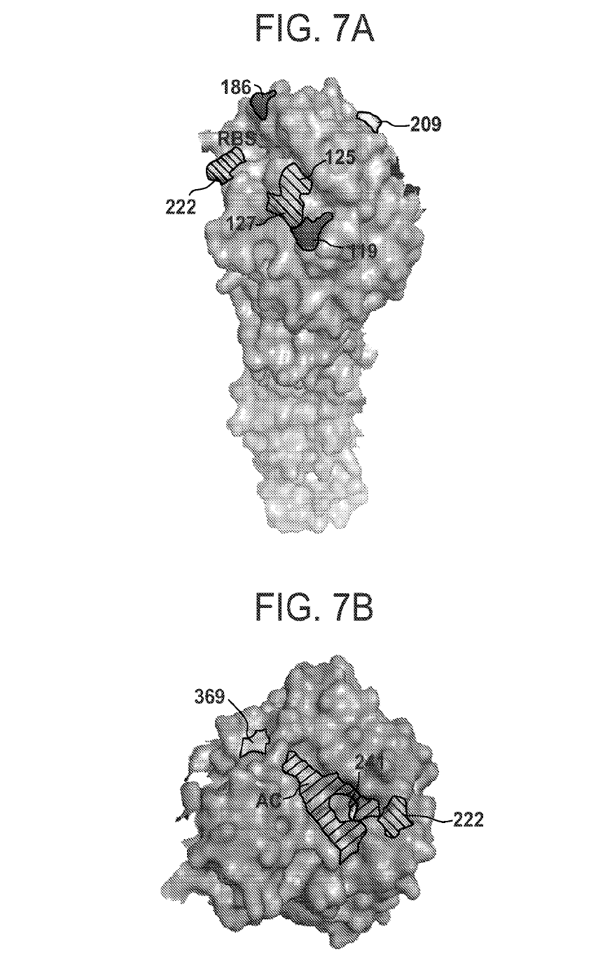

Overall, the HA N125D/D127E and D127E/K209E adaptation sites were demonstrated to be responsible for the high growth of A/Brisbane/10/2010 and A/New Hamsphire/2/2010 influenza strains. Introduction of these substitutions into the heterologous CA/09 ca virus HA could revert its poor growth. The HA residue 125 is located in the antigenic Sa domain and adjacent to the receptor binding site (RBS) (FIG. 7A). A/Brisbane/10/2010-like viruses containing a HA N125D showed high growth in eggs. The A/Brisbane/10/2010-like viruses having a H1 HA N125D change were initially detected in late April 2010 in clinical isolates from the Southern Hemisphere (Barr et al., 2010 Euro Surveill. 15:pii: 19692, 26). Although the Brisbane-like strains did not greatly differ in antigenicity from earlier, A/California/09 strains, they have been associated with several vaccine breakthrough infections and were identified in a number of fatal cases (Barr et al., 2010 Euro Surveill. 15:pii: 19692; Strengell et al., 2011 PLoS One 6:e25848). The D127E or K209E changes in A/New Hampshire/2/2010 resulted from egg adaptation. Changes in HA 127 and 209 have been detected in other circulating H1 influenza A viruses or following adaptation in mice (Chen et al., 2011 Virology 412:401-410; Robertson et al., 2011 Vaccine 29:1836-1843). These residues are located on the surface of the globular head (FIG. 7A). A mouse-adapted A/CA/04 having an HA with D127E was shown to be associated with a more virulent phenotype in mice (Ye et al., 2010 PLoS Pathog. 6:e1001145). The 209 residue is relatively distant from the RBS in the neighboring monomer in the HA trimer. A K209T change has been reported in some high-yield reassortants for inactivated influenza vaccines, however, a single K209T change did not greatly improve vaccine yield (Robertson et al., 2011 Vaccine 29:1836-1843).

Genetic signatures in the NA that contributed to vaccine virus growth in eggs included S222N, I241V, K369N separately or in combination. A/Gilroy/231/2011 grew particularly well when both the HA and NA proteins were altered. Amino acids at positions of 222, 241 and 369 (corresponding to N2 numbering 221, 240 and 372, respectively) were mainly responsible for the poor growth of Gil/11. These three residues are all around the NA catalytic site (FIG. 7B). The 369 residue is close to the conservative catalytic site R371 and both 369 and 222 are on the antigenic surface (Colman et al., 1989 p. 175-218. In R. Krug (ed.), The Influenza Viruses. Plenum Press, New York; Li et al., 2010 Nat Struct Mol Biol. 17:1266-1268). The K369 and 1241 in Gil/11 NA are conserved in the previous human seasonal H1N1 strains and most recent 2011/2012 H1N1pdm strains contain K369 and 1241, suggesting that the NA of the recent H1N1pdm strains may have adapted well in humans (Soundararajan et al., 2009 Nat Biotechnol. :6).

### 9. Discussion

While not wishing to be bound by theory it may be that the HA residue changes around the receptor binding site favor receptor binding in eggs or MDCK cells and the acidic surface changes in the HA further help virus release from infected cells to initiate efficient multi-cycle replication. A previous study (Chen et al., 2010 J. Virol. 84:44-51), in combination with the Examples provided herein, demonstrate that the amino acid substitutions of D222G, A186D, N125D, D127E and K209E in HA greatly improve virus growth in eggs or MDCK cells. Most of these changes are acidic residue changes. These negatively charged residues may cause repulsion of the negatively charged sialic acid receptor or cell membrane and increase virus particle release from MDCK cells without affecting viral entry and viral protein synthesis, as demonstrated by western blot and immunofluorescence assays.

It has also been hypothesized that egg adaptation changes in HA increased virus binding to α2,3-linked sialic acid improved virus replication in eggs (Nicolson et al., 2012 Vaccine 30:745-751; Robertson et al., 2011 Vaccine 29:1836-1843; Suphaphiphat et al., 2011 Virol J. 7:157); a D222G change increased virus binding to α2,3-linked sialic acid (Chen et al., 2010 J. Virol. 84:44-51; Chutinimitkul et al., 2010 J. Virol. 84:11802-11813). However, a receptor binding assay using resialylated red blood cells showed that viruses with N125D, D127E or K209E changes remain predominantly bound to α2,6-linked sialic acid receptors (data not shown), which is consistent with other glycan binding reports (Bradley et al, 2011, Virology 413:169-182; Chen et al., 2011 Virology 412:401-410; Xu et al., 2012 J Virol. 86:982-990). Possibly the current in vitro methods failed to detect the differences in the receptor binding caused by these changes.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

### TABLE OF SEQUENCES

SEQ ID NO: 1 depicts the amino acid sequence of HA polypeptide from A/California/07/09. Where X at 125=N or D; X at 127=D or E; X at 209=K or E.

SEQ ID NO: 2 depicts the amino acid sequence of HA polypeptide from A/Brisbane/10/10.

SEQ ID NO: 3 depicts the amino acid sequence of HA polypeptide from A/NewHampshire/2/10. Where X at 124=P or L; X at 125=N or D; X at 127=D or E; X at 209=K or E.

SEQ ID NO: 4 depicts the amino acid sequence of HA polypeptide from A/Gilroy/231/11. Where X at 125=Nor D; X at 127=D or E; X at 209=K or E.

SEQ ID NO: 5 depicts the amino acid sequence of the NA polypeptide from A/California/07/09.

SEQ ID NO: 6 depicts the amino acid sequence of the NA polypeptide from A/NewHampshire/2/10.

SEQ ID NO: 7 depicts the amino acid sequence of the NA polypeptide from A/Brisbane/10/10.

SEQ ID NO: 8 depicts the amino acid sequence of the NA polypeptide from A/Gilroy/231/11. Where X at 222=S or N; X at 241=I or V; X at 369=K or N

SEQ ID NO: 9 depicts the nucleotide sequence encoding the HA polypeptide of A/CA/07/09.

SEQ ID NO: 10 depicts the nucleic acid sequence encoding the HA from A/Brisbane/10/10.

SEQ ID NO: 11 depicts the nucleic acid sequence encoding the HA polypeptide from A/NewHampshire/2/10.

SEQ ID NO: 12 depicts the nucleic acid sequence encoding the HA polypeptide of A/Gilroy/231/11.

SEQ ID NO: 13 depicts the nucleic acid sequence encoding the NA polypeptide of A/Brisbane/10/10.

SEQ ID NO: 14 depicts the nucleic acid sequence encoding the NA polypeptide of A/NewHampshire/2/10.

SEQ ID NO: 15 depicts the nucleic acid sequence encoding the NA polypeptide of A/Gilroy/231/11.

SEQ ID NO: 16 depicts the nucleic acid sequence encoding the NA polypeptide of A/California/07/09.

SEQUENCES

A/California/07/09 HA
(SEQ ID NO: 1 wherein X at 125 = N; X at 127 = D;
X at 209 = K)
DTLCIGYHAN NSTDTVDTVL EKNVTVTHSV NLLEDKHNGK
LCKLRGVAPL HLGKCNIAGW ILGNPECESL STASSWSYIV
ETPSSDNGTC YPGDFIDYEE LREQLSSVSS FERFEIFPKT
SSWPXHXSNK GVTAACPHAG AKSFYKNLIW LVKKGNSYPK
LSKSYINDKG KEVLVLWGIH HPSTSADQQS LYQNADAYVF
VGSSRYSKXF KPEIAIRPKV RDQEGRMNYY WTLVEPGDKI
TFEATGNLVV PRYAFAMERN AGSGIIISDT PVHDCNTTCQ
TPKGAINTSL PFQNIHPITI GKCPKYVKST KLRLATGLRN
IPSIQSRGLF GAIAGFIEGG WTGMVDGWYG YHHQNEQGSG
YAADLKSTQN AIDEITNKVN SVIEKMNTQF TAVGKEFNHL
EKRIENLNKK VDDGFLDIWT YNAELLVLLE NERTLDYHDS
NVKNLYEKVR SQLKNNAKEI GNGCFEFYHK CDNTCMESVK
NGTYDYPKYS EEAKLNREEI DGVKLESTRI YQILAIYSTV
ASSLVLVVSL GAISFWMCSN GSLQCRICI
A/Brisbane/10/10 HA (SEQ ID NO: 2)
DTLCIGYHAN NSTDTVDTVL EKNVTVTHSV NLLEDKHNGK
LCKLRGVAPL HLGKCNIAGW ILGNPECESL STASSWSYIV
ETSSSDNGTC YPGDFIDYEE LREQLSSVSS FERFEIFPKT
SSWPDHESNK GVTAACPHAG AKSFYKNLIW LVKKGNSYPK
LSKSYINDKG KEVLVLWGIH HPSTSADQQS LYQNADAYVF
VGTSRYSKKF KPEIAIRPKV RDQEGRMNYY WTLVEPGDKI
TFEATGNLVV PRYAFAMERN AGSGIIISDT PVHDCNTTCQ
TPKGAINTSL PFQNIHPITI GKCPKYVKST KLRLATGLRN
VPSIQSRGLF GAIAGFIEGG WTGMVDGWYG YHHQNEQGSG
YAADLKSTQN AIDKITNKVN SVIEKMNTQF TAVGKEFNHL
EKRIENLNKK VDDGFLDIWT YNAELLVLLE NERTLDYHDS
NVKNLYEKVR SQLKNNAKEI GNGCFEFYHK CDNTCMESVK
NGTYDYPKYS EEAKLNREEI DGVKLESTRI YQILAIYSTV
ASSLVLVVSL GAISFWMCSN GSLQCRICI
A/NewHampshire/2/10 HA
(SEQ ID NO: 3 wherein X at 124 = P; X at 125 = N;
X at 127 = D; X at 209 = K)
DTLCIGYHAN NSTDTVDTVL EKNVTVTHSV NLLEDKHNGK
LCKLRGVAPL HLGKCNIAGW ILGNPECESL STASSWSYIV
ETSSSDNGTC YPGDFINYEE LREQLSSVSS FERFEIFPKT
SSWXXHXSNK GVTAACPHAG AKSFYKNLIW LVKKGNSYPK
LSKSYINDKG KEVLVLWGIH HPSTSADQQS LYQNADAYVF
VGTSRYSKXF KPEIAIRPKV RDQEGRMNYY WTLVEPGDKI
TFEATGNLVV PRYAFAMERN AGSGIIISDT PVHDCNTTCQ
TPKGAINTSL PFQNIHPITI GKCPKYVKST KLRLATGLRN
VPSIQSRGLF GAIAGFIEGG WTGMVDGWYG YHHQNEQGSG
YAADLKSTQN AIDKITNKVN SVIEKMNTQF TAVGKEFNHL
EKRIENLNKK VDDGFLDIWT YNAELLVLLE NERTLDYHDS
NVKNLYEKVR SQLKNNAKEI GNGCFEFYHK CDNTCMESVK
NGTYDYPKYS EEAKLNREEI DGVKLESTRI YQILAIYSTV
ASSLVLVVSL GAISFWMCSN GSLQCRICI
A/Gilroy/231/11 HA
(SEQ ID NO: 4 wherein X at 125 = N; X at 127 = D;
X at 209 = K)
DTLCIGYHAN NSTDTVDTVL EKNVTVTHSV NLLEDKHNGK
LCKLRGVAPL HLGKCNIAGW ILGNPECESL STASSWSYIV
ETSSSDNGTC YPGDFINYEE LREQLSSVSS FERFEIFPKT
SSWPXHXSNK GVTAACPHAG AKSFYKNLIW LVKKGNSYPK
LSKSYINDKG KEVLVLWGIH HPSTSADQQS LYQNADAYVF
VGTSKYSKXF KPEIAVRPKV RDQEGRMNYY WTLVEPGDKI
TFEATGNLLV PRYAFAMERN AGSGIIISDT PVHDCNTTCQ
TPEGAINTSL PFQNIHPITL GKCPKYVKST KLRLATGLRN
VPSIQSRGLF GAIAGFIEGG WTGMVDGWYG YHHQNEQGSG
YAADLKSTQN AIDKITNKVN SVIEKMNTQF TAVGKEFNHL
EKRIENLNKK VDDGFLDIWT YNAELLVLLE NERTLDYHDS
NVKNLYEKVR SQLKNNAKEI GNGCFEFYHK CDNTCMESVK
NGTYDYPKYS EEAKLNREEI DGVKLESTRI YQILAIYSTV
ASSLVLVVSL GAISFWMCSN GSLQCRICI
A/California/07/09 NA (SEQ ID NO: 5)
MNPNQKIITI GSVCMTIGMA NLILQIGNII SIWISHSIQL
GNQNQIETCN MNPNQKIITI SSVCMTIGMA NLILQIGNII
SIWISHSIQL GNQNQIETCN QSVITYENNT WVNQTYVNIS
NTNFAAGQSV VSVKLAGNSS LCPVSGWAIY SKDNSVRIGS
KGDVFVIREP FISCSPLECR TFFLTQGALL NDKHSNGTIK
DRSPYRTLMS CPIGEVPSPY NSRFESVAWS ASACHDGINW
LTIGISGPDN GAVAVLKYNG IITDTIKSWR NNILRTQESE
CACVNGSCFT VMTDGPSNGQ ASYKIFRIEK GKIVKSVEMN
APNYHYEECS CYPDSSEITC VCRDNWHGSN RPWVSFNQNL
EYQIGYICSG IFGDNPRPND KTGSCGPVSS NGANGVKGFS
FKYGNGVWIG RTKSISSRNG FEMIWDPNGW TGTDNNFSIK
QDIVGINEWS GYSGSFVQHP ELTGLDCIRP CFWVELIRGR
PKENTIWTSG SSISFCGVNS DTVGWSWPDG AELPFTIDK
A/NewHampshire/2/10 NA (SEQ ID NO: 6)
MNPNQKIITI SSVCMTIGMA NLILQIGNII SIWISHSIQL
GNQNQIETCN QSVITYENNT WVNQTYVNIS NTNFAAGQSV
VSVKLAGNSS LCPVSGWAIY SKDNSIRIGS KGDVFVIREP
FISCSPLECR TFFLTQGALL NDKHSNGTIK DRSPYRTLMS
CPIGEVPSPY NSRFESVAWS ASACHDGINW LTIGISGPDN
GAVAVLKYNG IITDTIKSWR NNILRTQESE CACVNGSCFT
VMTDGPSDGQ ASYKIFRIEK GKIVKSVEMN APNYHYEECS
CYPDSSEITC VCRDNWHGSN RPWVSFNQNL EYQIGYICSG
IFGDNPRPND KTGSCGPVSS NGANGVKGFS FKYGNGVWIG
RTKSISSRNG FEMIWDPNGW TGTDNNFSIK QDIVGINEWS
GYSGSFVQHP ELTGLDCIRP CFWVELIRGR PKENTIWTSG
SSISFCGVNS DTVGWSWPDG AELPFTIDK
A/Brisbane/10/10 NA (SEQ ID NO: 7)
MNPNQKIITI GSVCITIGMA NLILQIGNII SIWISHSIQL
GNQNQIETCN QSVITYENNT WVNQTYVNIS NTNFAAGQSV
VSVKLAGNSS LCPVSGWAIY SKDNSIRIGS KGDVFVIREP
FISCSPLECR TFFLTQGALL NDKHSNGTIK DRSPYRTLMS
CPIGEVPSPY NSRFESVAWS ASACHDGISW LTIGISGPDN
GAVAVLKYNG IITDTIKSWR NNILRTQESE CACVNGSCFT
VMTDGPSDGQ ASYKIFRIEK GKIVKSVEMN APNYHYEECS
CYPDSSEITC VCRDNWHGSN RPWVSFNQNL EYQIGYICSG
IFGDNPRPND KTGSCGPVSS NGANGVKGFS FKYGNGVWIG
RTKSISSRNG FEMIWDPNGW TGTDNNFSIK QDIVGINEWS
GYSGSFVQHP ELTGLDCIKP CFWVELIRGR PKENTIWTSG
SSISFCGVNS DTVGWSWPDG AELPFTIDK
A/Gilroy/231/11 NA
(SEQ ID NO: 8 wherein X at 222 = S; X at 241 = I;
X at 369 = K)
MNPNQKIITI GSVCMTIGMA NLILQIGNII SIWISHSIQL
GNQSQIETCN QSVITYENNT WVNQTYVNIS NTNFAAGQSV
VSVKLAGNSS LCPVSGWAIY SKDNSIRIGS KGDVFVIREP
FISCSPLECR TFFLTQGALL NDKHSNGTIK DRSPYRTLMS
CPIGEVPSPY NSRFESVAWS ASACHDGINW LTIGISGPDN
GAVAVLKYNG IITDTIKSWR NXILRTQESE CACVNGSCFT
XMTDGPSDGQ ASYKIFRIEK GKIVKSVEMN APNYHYEECS
CYPDSSEITC VCRDNWHGSN RPWVSFNQNL EYQIGYICSG
IFGDNPRPND KTGSCGPVSS NGANGVKGFS FKYGNGVWIG
RTKSISSRXG FEMIWDPNGW TGTDNNFSIK QDIVGINEWS
GYSGSFVQHP ELTGLDCIRP CFWVELIRGR PKENTIWTSG
SSMSFCGVNS DTVGWSWPDG AELPFTIDK
A/CA/07/09 HA (SEQ ID NO: 9)
AGCAAAAGCA GGGGAAAACA AAAGCAACAA AAATGAAGGC
AATACTAGTA GTTCTGCTAT ATACATTTGC AACCGCAAAT
GCAGACACAT TATGTATAGG TTATCATGCG AACAATTCAA
CAGACACTGT AGACACAGTA CTAGAAAAGA ATGTAACAGT
AACACACTCT GTTAACCTTC TAGAAGACAA GCATAACGGG
AAACTATGCA AACTAAGAGG GGTAGCCCCA TTGCATTTGG
GTAAATGTAA CATTGCTGGC TGGATCCTGG GAAATCCAGA
GTGTGAATCA CTCTCCACAG CAAGCTCATG GTCCTACATT
GTGGAAACAC CTAGTTCAGA CAATGGAACG TGTTACCCAG
GAGATTTCAT CGATTATGAG GAGCTAAGAG AGCAATTGAG
CTCAGTGTCA TCATTTGAAA GGTTTGAGAT ATTCCCCAAG
ACAAGTTCAT GGCCCAATCA TGACTCGAAC AAAGGTGTAA
CGGCAGCATG TCCTCATGCT GGAGCAAAAA GCTTCTACAA
AAATTTAATA TGGCTAGTTA AAAAAGGAAA TTCATACCCA
AAGCTCAGCA AATCCTACAT TAATGATAAA GGGAAAGAAG
TCCTCGTGCT ATGGGGCATT CACCATCCAT CTACTAGTGC
TGACCAACAA AGTCTCTATC AGAATGCAGA TGCATATGTT
TTTGTGGGGT CATCAAGATA CAGCAAGAAG TTCAAGCCGG
AAATAGCAAT AAGACCCAAA GTGAGGGATC AAGAAGGGAG
AATGAACTAT TACTGGACAC TAGTAGAGCC GGGAGACAAA
ATAACATTCG AAGCAACTGG AAATCTAGTG GTACCGAGAT
ATGCATTCGC AATGGAAAGA AATGCTGGAT CTGGTATTAT
CATTTCAGAT ACACCAGTCC ACGATTGCAA TACAACTTGT
CAAACACCCA AGGGTGCTAT AAACACCAGC CTCCCATTTC
AGAATATACA TCCGATCACA ATTGGAAAAT GTCCAAAATA
TGTAAAAAGC ACAAAATTGA GACTGGCCAC AGGATTGAGG
AATATCCCGT CTATTCAATC TAGAGGCCTA TTTGGGGCCA
TTGCCGGTTT CATTGAAGGG GGGTGGACAG GGATGGTAGA
TGGATGGTAC GGTTATCACC ATCAAAATGA GCAGGGGTCA
GGATATGCAG CCGACCTGAA GAGCACACAG AATGCCATTG
ACGAGATTAC TAACAAAGTA AATTCTGTTA TTGAAAAGAT
GAATACACAG TTCACAGCAG TAGGTAAAGA GTTCAACCAC
CTGGAAAAAA GAATAGAGAA TTTAAATAAA AAAGTTGATG
ATGGTTTCCT GGACATTTGG ACTTACAATG CCGAACTGTT
GGTTCTATTG GAAAATGAAA GAACTTTGGA CTACCACGAT
TCAAATGTGA AGAACTTATA TGAAAAGGTA AGAAGCCAGC
TAAAAAACAA TGCCAAGGAA ATTGGAAACG GCTGCTTTGA
ATTTTACCAC AAATGCGATA ACACGTGCAT GGAAAGTGTC
AAAAATGGGA CTTATGACTA CCCAAAATAC TCAGAGGAAG
CAAAATTAAA CAGAGAAGAA ATAGATGGGG TAAAGCTGGA
ATCAACAAGG ATTTACCAGA TTTTGGCGAT CTATTCAACT
GTCGCCAGTT CATTGGTACT GGTAGTCTCC CTGGGGGCAA
TCAGTTTCTG GATGTGCTCT AATGGGTCTC TACAGTGTAG
AATATGTATT TAACATTAGG ATTTCAGAAG CATGAGAAAA
ACACCCTTGT TT
A/Brisbane/10/10 HA (SEQ ID NO: 10)
ATGAAGGC AATACTAGTA
GTTCTGCTAT ATACATTTGC AACCGCAAAT GCAGACACAT
TATGTATAGG TTATCATGCG AACAATTCAA CAGACACTGT
AGACACAGTA CTAGAAAAGA ATGTAACAGT AACACACTCT
GTTAACCTTC TAGAAGACAA GCATAACGGG AAATTATGCA
AACTAAGAGG GGTAGCCCCA TTGCATTTGG GTAAATGTAA
CATTGCTGGC TGGATCCTGG GAAATCCAGA GTGTGAATCA
CTCTCCACAG CAAGCTCATG GTCCTACATT GTGGAAACAT
CTAGTTCAGA CAATGGAACG TGTTACCCAG GAGATTTCAT
CGATTATGAG GAGCTAAGAG AACAATTGAG CTCAGTGTCA
TCATTTGAAA GGTTTGAGAT ATTCCCCAAG ACAAGTTCAT
GGCCCGATCA TGAATCGAAC AAAGGTGTAA CGGCAGCATG
TCCTCATGCT GGAGCAAAAA GCTTCTACAA AAATTTAATA
TGGCTAGTTA AAAAAGGAAA TTCATACCCA AAGCTCAGCA
AATCCTACAT TAATGATAAA GGGAAAGAAG TCCTCGTGCT
ATGGGGCATT CACCATCCAT CTACTAGTGC TGACCAACAA
AGTCTCTATC AGAATGCAGA TGCATATGTT TTTGTGGGGA
CATCAAGATA CAGCAAGAAG TTCAAGCCGG AAATAGCAAT
AAGACCCAAA GTGAGGGATC AAGAAGGGAG AATGAACTAT
TACTGGACAC TAGTAGAGCC GGGAGACAAA ATAACATTCG
AAGCAACTGG AAATCTAGTG GTACCGAGAT ATGCATTCGC
AATGGAAAGA AATGCTGGAT CTGGTATTAT CATTTCAGAT
ACACCAGTCC ACGATTGCAA TACAACTTGT CAGACACCCA
AGGGTGCTAT AAACACCAGC CTCCCATTTC AGAATATACA
TCCGATCACA ATTGGAAAAT GTCCAAAATA TGTAAAAAGC
ACAAAATTGA GACTGGCCAC AGGATTGAGG AATGTCCCGT
CTATTCAATC TAGAGGCCTA TTTGGGGCCA TTGCCGGTTT
CATTGAAGGG GGGTGGACAG GGATGGTAGA TGGATGGTAC
GGTTATCACC ATCAAAATGA GCAGGGGTCA GGATATGCAG
CCGACCTGAA GAGCACACAG AATGCCATTG ACAAGATTAC
TAACAAAGTA AATTCTGTTA TTGAAAAGAT GAATACACAG
TTCACAGCAG TAGGTAAAGA GTTCAACCAC CTGGAAAAAA
GAATAGAGAA TTTAAATAAA AAAGTTGATG ATGGTTTCCT
GGACATTTGG ACTTACAATG CCGAACTGTT GGTTCTATTG
GAAAATGAAA GAACTTTGGA CTACCACGAT TCAAATGTGA
AGAACTTATA TGAAAAGGTA AGAAGCCAGT TAAAAAACAA
TGCCAAGGAA ATTGGAAACG GCTGCTTTGA ATTTTACCAC
AAATGCGATA ACACGTGCAT GGAAAGTGTC AAAAATGGGA
CTTATGACTA CCCAAAATAC TCAGAGGAAG CAAAATTAAA
CAGAGAAGAA ATAGATGGGG TAAAGCTGGA ATCAACAAGG
ATTTACCAGA TTTTGGCGAT CTATTCAACT GTCGCCAGTT
CATTGGTACT GGTAGTCTCC CTGGGGGCAA TCAGTTTCTG
GATGTGCTCT AATGGGTCTC TACAGTGTAG AATATGTATT
A/NewHampshire/2/10 HA (SEQ ID NO: 11)
ATGAAGGC AATACTAGTA
GTTCTGCTAT ATACATTTGC AACCGCAAAT GCAGACACAT
TATGTATAGG TTATCATGCG AACAATTCAA CAGACACTGT
AGACACAGTA CTAGAAAAGA ATGTAACAGT AACACACTCT
GTTAACCTTC TAGAAGACAA GCATAACGGG AAACTATGCA
AACTAAGAGG GGTAGCCCCA TTGCATTTGG GTAAATGTAA
CATTGCTGGC TGGATCCTGG GAAATCCAGA GTGTGAATCA
CTCTCCACAG CAAGCTCATG GTCCTACATT GTGGAAACAT
CTAGTTCAGA CAATGGAACG TGTTACCCAG GAGATTTCAT
CAATTATGAG GAGCTAAGAG AGCAATTGAG CTCAGTGTCA
TCATTTGAAA GGTTTGAGAT ATTCCCCAAG ACAAGTTCAT
GGCCCAATCA TGACTCGAAC AAAGGTGTAA CGGCAGCATG
TCCTCATGCT GGAGCAAAAA GCTTCTACAA AAATTTAATA
TGGCTAGTTA AAAAAGGAAA TTCATACCCA AAGCTCAGCA
AATCCTACAT TAATGATAAA GGGAAAGAAG TCCTCGTACT
ATGGGGCATT CACCATCCAT CTACTAGTGC TGACCAACAA
AGTCTCTATC AGAATGCAGA TGCATATGTT TTTGTGGGGA
CATCAAGATA CAGCAAGAAG TTCAAGCCGG AAATAGCAAT
AAGACCCAAA GTGAGGGATC AAGAAGGGAG AATGAACTAT
TACTGGACAC TAGTAGAGCC GGGAGACAAA ATAACATTCG
AAGCAACTGG AAATCTAGTG GTACCGAGAT ATGCATTCGC
AATGGAAAGA AATGCTGGAT CTGGTATTAT CATCTCAGAT
ACACCAGTCC ACGATTGCAA TACAACTTGT CAGACACCCA
AGGGTGCTAT AAACACCAGC CTCCCATTTC AGAATATACA
TCCGATCACA ATTGGAAAAT GTCCAAAATA TGTAAAAAGC
ACAAAATTGA GACTGGCCAC AGGATTGAGG AATGTCCCGT
CTATTCAATC TAGAGGCCTA TTTGGGGCCA TTGCCGGTTT
CATTGAAGGG GGGTGGACAG GGATGGTAGA TGGATGGTAC
GGTTATCACC ATCAAAATGA GCAGGGGTCA GGATATGCAG
CCGACCTGAA GAGCACACAG AATGCCATTG ACAAGATTAC
TAACAAAGTA AATTCTGTTA TTGAAAAGAT GAATACACAG
TTCACAGCAG TAGGTAAAGA GTTCAACCAC CTGGAAAAAA
GAATAGAGAA TTTAAATAAA AAAGTTGATG ATGGTTTCCT
GGACATTTGG ACTTACAATG CCGAACTGTT GGTTCTATTG
GAAAATGAAA GAACTTTGGA CTACCACGAT TCAAATGTGA
AGAACTTATA TGAAAAGGTA AGAAGCCAGT TAAAAAACAA
TGCCAAGGAA ATTGGAAACG GCTGCTTTGA ATTTTACCAC
AAATGCGATA ACACGTGCAT GGAAAGTGTC AAAAATGGGA
CTTATGACTA CCCAAAATAC TCAGAGGAAG CAAAATTAAA
CAGAGAAGAA ATAGATGGGG TAAAGCTGGA ATCAACAAGG
ATTTACCAGA TTTTGGCGAT CTATTCAACT GTCGCCAGTT
CATTGGTACT GGTAGTCTCC CTGGGGGCAA TCAGTTTCTG
GATGTGCTCT AATGGGTCTC TACAGTGTAG AATATGTATT TAA
A/Gilroy/231/11 HA (SEQ ID NO: 12)
GACACAT TATGTATAGG
TTATCATGCG AACAATTCAA CAGACACTGT AGACACAGTA
CTAGAAAAGA ATGTAACAGT AACACACTCT GTTAACCTTC
TAGAAGACAA GCATAACGGG AAACTATGCA AACTGAGAGG
GGTAGCCCCA TTGCATTTGG GTAAATGTAA CATTGCTGGC
TGGATCCTGG GAAATCCAGA GTGTGAATCA CTCTCCACAG
CAAGCTCATG GTCCTACATT GTGGAAACAT CTAGTTCAGA
CAATGGAACG TGTTACCCAG GAGATTTCAT CAATTATGAG
GAGCTAAGAG AGCAATTGAG CTCAGTGTCA TCATTTGAAA
GGTTTGAGAT ATTCCCCAAG ACAAGTTCAT GGCCCAATCA
TGACTCGAAC AAAGGTGTAA CGGCAGCATG TCCTCATGCT
GGAGCAAAAA GCTTCTACAA AAATTTAATA TGGCTAGTTA
AAAAAGGAAA TTCATACCCA AAGCTCAGCA AATCCTACAT
TAACGATAAA GGGAAAGAAG TCCTCGTGCT GTGGGGAATT
CACCATCCAT CTACTAGTGC TGACCAACAA AGTCTCTATC
AGAATGCAGA TGCATATGTT TTTGTGGGGA CATCAAAATA
CAGCAAGAAA TTCAAGCCGG AAATAGCAGT AAGACCCAAA
GTGAGGGATC AAGAAGGGAG AATGAACTAT TACTGGACAC
TAGTAGAGCC GGGAGACAAA ATAACATTCG AAGCAACTGG
AAATCTATTG GTACCGAGAT ATGCATTCGC AATGGAAAGA
AATGCTGGAT CTGGTATTAT CATTTCAGAT ACACCAGTCC
ACGATTGCAA TACAACTTGT CAAACACCCG AGGGTGCTAT
AAACACCAGC CTCCCATTTC AGAATATACA TCCGATCACA
CTTGGAAAAT GTCCAAAATA TGTAAAAAGC ACAAAATTGA
GACTGGCCAC AGGATTGAGG AATGTCCCGT CTATTCAATC
TAGAGGCCTA TTTGGGGCCA TTGCCGGTTT CATTGAAGGG
GGGTGGACAG GGATGGTAGA TGGATGGTAC GGTTATCACC
ATCAAAATGA GCAGGGGTCA GGATATGCAG CCGACCTGAA
GAGCACACAG AATGCCATTG ACAAGATTAC TAACAAAGTA
AATTCTGTTA TTGAAAAGAT GAATACACAG TTCACAGCAG
TAGGTAAAGA GTTCAACCAC CTGGAAAAAA GAATAGAGAA
TTTAAATAAA AAGGTTGATG ATGGTTTCCT GGACATTTGG
ACTTACAATG CCGAACTGTT GGTTCTATTG GAAAATGAAA
GAACTTTGGA CTACCACGAT TCAAATGTGA AAAACTTATA
TGAAAAGGTA AGAAGCCAGT TAAAAAACAA TGCCAAAGAA
ATTGGAAACG GCTGCTTTGA ATTTTACCAC AAATGCGATA
ACACGTGCAT GGAAAGTGTC AAAAATGGGA CTTATGACTA
CCCAAAATAC TCAGAGGAAG CAAAATTAAA CAGAGAAGAA
ATAGATGGGG TAAAGCTGGA ATCAACAAGG ATTTACCAGA
TTTTGGCGAT CTATTCAACT GTCGCCAGTT CATTGGTACT
GGTAGTCTCC CTGGGGGCAA TCAGTTTCTG GATGTGCTCT
AATGGGTCTC TACAGTGTAG AATATGTATT TAACATTAGG
ATTTCAGAAG CATGAGAAAA ACACCCTTGT TTCTACTAAT
ACGAGGCAG
A/Brisbane/10/10 NA (SEQ ID NO: 13)
ATGAATCCAA ACCAAAAGAT AATAACCATT
GGTTCGGTCT GTATAACAAT TGGAATGGCT AACTTAATAT
TACAAATTGG AAACATAATC TCAATATGGA TTAGCCACTC
AATTCAACTT GGGAATCAAA ATCAGATTGA AACATGCAAT
CAAAGCGTCA TTACTTATGA AAACAACACT TGGGTAAATC
AGACATATGT TAACATCAGC AACACCAACT TTGCTGCTGG
ACAGTCAGTG GTTTCCGTGA AATTAGCGGG CAATTCCTCT
CTCTGCCCTG TTAGTGGATG GGCTATATAC AGTAAAGACA
ACAGTATAAG AATCGGTTCC AAGGGGGATG TGTTTGTCAT
AAGGGAACCA TTCATATCAT GCTCCCCCTT GGAATGCAGA
ACCTTCTTCT TGACTCAAGG GGCCTTGCTA AATGACAAAC
ATTCCAATGG AACCATTAAA GACAGGAGCC CATATCGAAC
CCTAATGAGC TGTCCTATTG GTGAAGTTCC CTCTCCATAC
AACTCAAGAT TTGAGTCAGT CGCTTGGTCA GCAAGTGCTT
GTCATGATGG CATCAGTTGG CTAACAATTG GAATTTCTGG
CCCAGACAAT GGGGCAGTGG CTGTGTTAAA GTACAACGGC
ATAATAACAG ACACTATCAA GAGTTGGAGA AACAATATAT
TGAGAACACA AGAGTCTGAA TGTGCATGTG TAAATGGTTC
TTGTTTTACT GTAATGACCG ATGGACCAAG TGATGGACAG
GCCTCATACA AGATCTTCAG AATAGAAAAG GGAAAGATAG
TCAAATCAGT CGAAATGAAT GCCCCTAATT ATCACTATGA
GGAATGCTCC TGTTATCCTG ATTCTAGTGA AATCACATGT
GTGTGCAGGG ATAACTGGCA TGGCTCGAAT CGACCGTGGG
TGTCTTTCAA CCAGAATCTG GAATATCAGA TAGGATACAT
ATGCAGTGGG ATTTTCGGAG ACAATCCACG CCCTAATGAT
AAGACAGGCA GTTGTGGTCC AGTATCGTCT AATGGAGCAA
ATGGAGTAAA AGGATTTTCA TTCAAATACG GCAATGGTGT
TTGGATAGGG AGAACTAAAA GCATTAGTTC AAGAAACGGT
TTTGAGATGA TTTGGGATCC GAACGGATGG ACTGGGACAG
ACAATAACTT CTCAATAAAG CAAGATATCG TAGGAATAAA
TGAGTGGTCA GGATATAGCG GGAGTTTTGT TCAGCATCCA
GAACTAACAG GGCTGGATTG TATAAAACCT TGCTTCTGGG
TTGAACTAAT CAGAGGGCGA CCCAAAGAGA ACACAATCTG
GACTAGCGGG AGCAGCATAT CCTTTTGTGG TGTAAACAGT
GACACTGTGG GTTGGTCTTG GCCAGACGGT GCTGAGTTGC
CATTTACCAT TGACAAGTAA
A/NewHampshire/2/10_NA (SEQ ID NO: 14)
ATGAATCCAA ACCAAAAGAT AATAACCATT
AGTTCGGTCT GTATGACAAT TGGAATGGCT AACTTAATAT
TACAAATTGG AAACATAATC TCAATATGGA TTAGCCACTC
AATTCAACTT GGGAATCAAA ATCAGATTGA AACATGCAAT
CAAAGCGTCA TTACTTATGA AAACAACACT TGGGTAAATC
AGACATATGT TAACATCAGC AACACCAACT TTGCTGCTGG
ACAGTCAGTG GTTTCCGTGA AATTAGCGGG CAATTCCTCT
CTCTGCCCTG TTAGTGGATG GGCTATATAC AGTAAAGACA
ACAGTATAAG AATCGGTTCC AAGGGGGATG TGTTTGTCAT
AAGGGAACCA TTCATATCAT GCTCCCCCTT GGAATGCAGA
ACCTTCTTCT TGACTCAAGG GGCCTTGCTA AATGACAAAC
ATTCCAATGG AACCATTAAA GACAGGAGCC CATATCGAAC
CCTAATGAGC TGTCCTATTG GTGAAGTTCC CTCTCCATAC
AACTCAAGAT TTGAGTCAGT CGCTTGGTCA GCAAGTGCTT
GTCATGATGG CATCAATTGG CTAACAATTG GAATTTCTGG
CCCAGACAAT GGGGCAGTGG CTGTGTTAAA GTACAACGGC
ATAATAACAG ACACTATCAA GAGTTGGAGA AACAATATAT
TGAGAACACA AGAGTCTGAA TGTGCATGTG TAAATGGTTC
TTGCTTTACT GTAATGACCG ATGGACCAAG TGATGGACAG
GCCTCATACA AGATCTTCAG AATAGAAAAG GGAAAGATAG
TCAAATCAGT CGAAATGAAT GCCCCTAATT ATCACTATGA
GGAATGCTCC TGTTATCCTG ATTCTAGTGA AATCACATGT
GTGTGCAGGG ATAACTGGCA TGGCTCGAAT CGACCGTGGG
TGTCTTTCAA CCAGAATCTG GAATATCAGA TAGGATACAT
ATGCAGTGGG ATTTTCGGAG ACAATCCACG CCCTAATGAT
AAGACAGGCA GTTGTGGTCC AGTATCGTCT AATGGAGCAA
ATGGAGTAAA AGGATTTTCA TTCAAATACG GCAATGGTGT
TTGGATAGGG AGAACTAAAA GCATTAGTTC AAGAAACGGT
TTTGAGATGA TTTGGGATCC GAACGGATGG ACTGGGACAG
ACAATAACTT CTCAATAAAG CAAGATATCG TAGGAATAAA
TGAGTGGTCA GGATATAGCG GGAGTTTTGT TCAGCATCCA
GAACTAACAG GGCTGGATTG TATAAGACCT TGCTTCTGGG
TTGAACTAAT CAGAGGGCGA CCCAAAGAGA ACACAATCTG
GACTAGCGGG AGCAGCATAT CCTTTTGTGG TGTAAACAGT
GACACTGTGG GTTGGTCTTG GCCAGACGGT GCTGAGTTGC
CATTTACCAT TGACAAGTAA
A/Gilroy /231/11 NA (SEQ ID NO: 15)
ATGAATCCAA ACCAAAAGAT AATAACCATT
GGTTCGGTCT GTATGACAAT TGGAATGGCT AACTTAATAT
TACAAATTGG AAACATAATC TCAATATGGA TTAGCCACTC
AATTCAACTT GGGAATCAAA GTCAGATTGA AACATGTAAT
CAAAGCGTCA TTACTTATGA AAACAACACT TGGGTAAATC
AGACATATGT TAACATCAGC AACACCAACT TTGCTGCTGG
ACAGTCAGTG GTTTCCGTGA AATTAGCGGG CAATTCCTCT
CTCTGCCCTG TTAGTGGATG GGCTATATAC AGTAAAGACA
ACAGTATAAG AATCGGTTCC AAGGGGGATG TGTTTGTCAT
AAGGGAACCA TTCATATCAT GCTCCCCCTT GGAATGCAGA
ACCTTCTTCT TGACTCAAGG GGCCTTGCTA AATGACAAAC
ATTCCAATGG AACCATTAAA GACAGGAGCC CATATCGAAC
CCTAATGAGC TGTCCTATTG GTGAAGTTCC CTCTCCATAC
AACTCAAGAT TTGAGTCAGT CGCTTGGTCA GCAAGTGCTT
GTCATGATGG CATCAATTGG CTAACAATTG GAATTTCTGG
CCCAGACAAT GGGGCAGTGG CTGTGTTAAA GTACAACGGC
ATAATAACAG ACACTATCAA GAGTTGGAGA AACAGTATAT
TGAGAACACA AGAGTCTGAA TGTGCATGTG TAAATGGTTC
TTGCTTTACC ATAATGACCG ATGGACCAAG TGATGGACAG
GCCTCATACA AGATCTTCAG AATAGAAAAG GGAAAAATAG
TCAAATCAGT CGAAATGAAT GCCCCTAATT ATCACTATGA
GGAATGCTCC TGTTATCCTG ATTCTAGTGA AATCACTTGT
GTGTGCAGGG ATAACTGGCA TGGCTCGAAT CGACCGTGGG
TGTCTTTCAA CCAGAATCTG GAATACCAGA TAGGATACAT
ATGCAGTGGG ATTTTCGGAG ACAATCCACG CCCTAATGAT
AAGACAGGCA GTTGTGGTCC AGTATCGTCT AATGGAGCAA
ATGGAGTAAA AGGATTTTCA TTCAAATACG GCAATGGTGT
TTGGATAGGG AGAACTAAAA GCATTAGTTC AAGAAAAGGT
TTTGAGATGA TTTGGGATCC AAACGGATGG ACTGGGACAG
ACAATAACTT CTCAATAAAG CAAGATATCG TAGGAATAAA
TGAGTGGTCA GGATATAGCG GGAGTTTTGT TCAGCATCCA
GAACTAACAG GGCTGGATTG TATAAGACCT TGCTTCTGGG
TTGAACTAAT CAGAGGGCGA CCCAAAGAGA ACACAATCTG
GACTAGCGGG AGCAGCATGT CCTTTTGTGG TGTAAACAGT
GACACTGTGG GTTGGTCTTG GCCAGACGGT GCTGAGTTGC
CATTTACCAT TGACAAGTAA TTTGTTCAAA AAACTCC
A/California/07/09_NA (SEQ ID NO: 16)
AGCAAAAGCA GGAGTTTAAA ATGAATCCAA ACCAAAAGAT
AATAACCATT GGTTCGGTCT GTATGACAAT TGGAATGGCT
AACTTAATAT TACAAATTGG AAACATAATC TCAATATGGA
TTAGCCACTC AATTCAACTT GGGAATCAAA ATCAGATTGA
AACATGCAAT CAAAGCGTCA TTACTTATGA AAACAACACT
TGGGTAAATC AGACATATGT TAACATCAGC AACACCAACT
TTGCTGCTGG ACAGTCAGTG GTTTCCGTGA AATTAGCGGG
CAATTCCTCT CTCTGCCCTG TTAGTGGATG GGCTATATAC
AGTAAAGACA ACAGTGTAAG AATCGGTTCC AAGGGGGATG
TGTTTGTCAT AAGGGAACCA TTCATATCAT GCTCCCCCTT
GGAATGCAGA ACCTTCTTCT TGACTCAAGG GGCCTTGCTA
AATGACAAAC ATTCCAATGG AACCATTAAA GACAGGAGCC
CATATCGAAC CCTAATGAGC TGTCCTATTG GTGAAGTTCC
CTCTCCATAC AACTCAAGAT TTGAGTCAGT CGCTTGGTCA
GCAAGTGCTT GTCATGATGG CATCAATTGG CTAACAATTG
GAATTTCTGG CCCAGACAAT GGGGCAGTGG CTGTGTTAAA
GTACAACGGC ATAATAACAG ACACTATCAA GAGTTGGAGA
AACAATATAT TGAGAACACA AGAGTCTGAA TGTGCATGTG
TAAATGGTTC TTGCTTTACT GTAATGACCG ATGGACCAAG
TAATGGACAG GCCTCATACA AGATCTTCAG AATAGAAAAG
GGAAAGATAG TCAAATCAGT CGAAATGAAT GCCCCTAATT
ATCACTATGA GGAATGCTCC TGTTATCCTG ATTCTAGTGA
AATCACATGT GTGTGCAGGG ATAACTGGCA TGGCTCGAAT
CGACCGTGGG TGTCTTTCAA CCAGAATCTG GAATATCAGA
TAGGATACAT ATGCAGTGGG ATTTTCGGAG ACAATCCACG
CCCTAATGAT AAGACAGGCA GTTGTGGTCC AGTATCGTCT
AATGGAGCAA ATGGAGTAAA AGGGTTTTCA TTCAAATACG
GCAATGGTGT TTGGATAGGG AGAACTAAAA GCATTAGTTC
AAGAAACGGT TTTGAGATGA TTTGGGATCC GAACGGATGG
ACTGGGACAG ACAATAACTT CTCAATAAAG CAAGATATCG
TAGGAATAAA TGAGTGGTCA GGATATAGCG GGAGTTTTGT
TCAGCATCCA GAACTAACAG GGCTGGATTG TATAAGACCT
TGCTTCTGGG TTGAACTAAT CAGAGGGCGA CCCAAAGAGA
ACACAATCTG GACTAGCGGG AGCAGCATAT CCTTTTGTGG
TGTAAACAGT GACACTGTGG GTTGGTCTTG GCCAGACGGT
GCTGAGTTGC CATTTACCAT TGACAAGTAA TTTGTTCAAA
AAACTCCTTG TTTCTACT

What is claimed is:

1. A recombinant reassortant influenza virus comprising a first genome segment encoding a hemagglutinin polypeptide, wherein the hemagglutinin polypeptide comprises the amino acid sequence as shown in SEQ ID NO:1, SEQ ID NO:3, or SEQ ID NO:4.

2. A recombinant reassortant influenza virus comprising a first genome segment encoding a hemagglutinin polypeptide, wherein the hemagglutinin polypeptide comprises:
a leucine at amino acid residue position 124; or
an aspartic acid at amino acid residue position 125; or
a glutamic acid at amino acid residue position 127; or
a glutamic acid at amino acid residue position 209; or
a leucine at amino acid residue position 124 and a glutamic acid at amino acid residue position 209; or
an aspartic acid at amino acid residue position 125 and a glutamic acid at amino acid residue position 127; or
an aspartic acid at amino acid residue position 125 and a glutamic acid at amino acid residue position 209; or
a glutamic acid at amino acid residue position 127 and a glutamic acid at amino acid residue position 209; or
an aspartic acid at amino acid residue position 125, a glutamic acid at amino acid residue position 127, and a glutamic acid at amino acid residue position 209; or
a leucine at amino acid residue position 124, a glutamic acid at amino acid residue position 127, and a glutamic acid at amino acid residue position 209; or

[symbol]

3. The recombinant reassortant influenza virus of claim 1 or claim 2, further comprising a second genome segment encoding a neuraminidase polypeptide, wherein the neuraminidase polypeptide comprises:
an asparagine at amino acid residue position 222; or
a valine at amino acid residue position 241; or
an asparagine at amino acid residue position 369; or
an asparagine at amino acid residue position 222 and an asparagine at amino acid residue position 369; or
a valine at amino acid residue position 241 and an asparagine at amino acid residue position 369; or
an asparagine at amino acid residue position 222, a valine at amino acid residue 241, and an asparagine at amino acid residue position 369.

4. The recombinant reassortant influenza virus of any one of claims 1 to 3 comprising further six internal genome segments of an influenza virus having phenotypic characteristics of one or more of attenuation, temperature sensitivity, and cold-adaptation.

5. The recombinant reassortant influenza virus of any one of claims 1 to 4, wherein the hemagglutinin polypeptide comprises the amino acid sequence as shown in SEQ ID NO:1; and wherein
amino acid at position 125 is aspartic acid; and
amino acid at position 127 is glutamic acid; and
amino acid at position 209 is glutamic acid.

6. The recombinant reassortant influenza virus of any one of claims 1 to 4, wherein the hemagglutinin polypeptide comprises the amino acid sequence as shown in SEQ ID NO:3; and wherein
amino acid at position 125 is aspartic acid; and
amino acid at position 127 is glutamic acid; and
amino acid at position 209 is glutamic acid.

7. The recombinant reassortant influenza virus of any one of claims 1 to 4, wherein the hemagglutinin polypeptide comprises the amino acid sequence as shown in SEQ ID NO:4; and wherein
amino acid at position 125 is aspartic acid; and
amino acid at position 127 is glutamic acid; and
amino acid at position 209 is glutamic acid.

8. The recombinant reassortant influenza virus of any one of claims 1 to 7, wherein the neuroaminidase polypeptide comprises the amino acid sequence as shown in SEQ ID NO:8; and wherein
amino acid at position 222 is asparagine; and
amino acid at position 241 is valine; and
amino acid at position 369 is asparagine.

9. The recombinant reassortant influenza virus of any one of claims 5 to 8, wherein the six internal genome segments are of influenza virus A/Ann Arbor/6/60.

10. The recombinant reassortant influenza virus of any one of claims 5 to 8, wherein the six internal genome segments are of influenza virus A/Puerto Rico/8/34.

11. The recombinant reassortant influenza virus of any one of claims 1 to 10, wherein the reassortant influenza virus has been inactivated.

12. The recombinant reassortant influenza virus of any of claims 1 to 11, wherein the reassortant influenza virus is live attenuated.

13. An immunogenic composition comprising the recombinant influenza virus of any one of claims 1 to 12.

14. The immunogenic composition of claim 13, comprising a recombinant influenza virus comprising H3N2 influenza A strain HA and NA antigens, a recombinant influenza virus comprising Yamagata influenza B strain HA and NA antigens, and a recombinant influenza virus comprising Victoria influenza B strain HA and NA antigens.

15. A method of producing a recombinant reassortant influenza virus comprising:
(a) introducing a plurality of vectors into a population of host cells capable of supporting replication of influenza viruses, which plurality of vectors comprises at least 6 internal genome segments of a first influenza strain, and a first genome segment which encodes a hemagglutinin polypeptide comprising the amino acid sequence as shown in SEQ ID NO:1, SEQ ID NO:3, or SEQ ID NO:4;
(b) culturing the population of host cells to amplify the recombinant reassortant influenza virus; and
(c) recovering the recombinant reassortant influenza virus from the population of host cells.

16. The method of claim 15, wherein the hemagglutinin polypeptide comprising the amino acid sequence of SEQ ID NO:1 comprises:
an aspartic acid at amino acid residue position 125; or
a glutamic acid at amino acid residue position 127; or
a glutamic acid at amino acid residue position 209; or
an aspartic acid at amino acid residue position 125 and a glutamic acid at amino acid residue position 127; or
an aspartic acid at amino acid residue position 125 and a glutamic acid at amino acid residue position 209; or
a glutamic acid at amino acid residue position 127 and a glutamic acid at amino acid residue position 209; or
an aspartic acid at amino acid residue position 125, a glutamic acid at amino acid residue position 127, and a glutamic acid at amino acid residue position 209.

17. The method of claim 15, wherein the hemagglutinin polypeptide comprising the amino acid sequence of SEQ ID NO:3 comprises:
a leucine at amino acid residue position 124; or
an aspartic acid at amino acid residue position 125; or
a glutamic acid at amino acid residue position 127; or
a glutamic acid at amino acid residue position 209; or
a leucine at amino acid residue position 124 and a glutamic acid at amino acid residue position 209; or
an aspartic acid at amino acid residue position 125 and a glutamic acid amino acid residue position 209; or
a glutamic acid at amino acid residue position 127 and a glutamic acid at amino acid residue position 209; or
a leucine at amino acid residue position 124, a glutamic acid at amino acid residue position 127, and a glutamic acid at amino acid residue position 209; or
an aspartic acid at amino acid residue position 125, a glutamic acid at amino acid residue 127, and a glutamic acid at amino acid residue position 209.

18. The method of claim 15, wherein the hemagglutinin polypeptide comprising the amino acid sequence of SEQ ID NO: 4 comprises:
an aspartic acid at amino acid residue position 125; or
a glutamic acid at amino acid residue position 127; or
a glutamic acid at amino acid residue position 209; or
an aspartic acid at amino acid residue position 125 and a glutamic acid at amino acid position 127; or
a glutamic acid at amino acid residue position 127 and a glutamic acid at amino acid residue position 209; or
an aspartic acid at amino acid residue position 125 and a glutamic acid at amino acid position 209; or
an aspartic acid at amino acid residue position 125, a glutamic acid at amino acid residue position 127, and a glutamic acid at amino acid residue position 209.

19. The method of any one of claims 15 to 18, wherein the plurality of vectors of step (a) comprises a second genome segment which encodes a neuraminidase polypeptide comprising the amino acid sequence of SEQ ID NO:5, or SEQ ID NO:7, or SEQ ID NO:8.

20. The method of claim 19 wherein the neuraminidase polypeptide comprises the amino acid sequence of SEQ ID NO:8.

21. The method of claim 20, wherein the neuraminidase polypeptide as shown in SEQ ID NO:8 comprises:
an asparagine at amino acid residue position 222; or
a valine at amino acid residue position 241; or
an asparagine at amino acid residue position 369; or
an asparagine at amino acid residue position 222 and an asparagine at amino acid residue position 369; or
a valine at amino acid residue position 241 and an asparagine at amino acid residue position 369; or
an asparagine at amino acid residue position 222, a valine at amino acid residue 241, and an asparagine at amino acid residue position 369.


SEQUENCE LISTING (16 sequences)

SEQ ID NO: 1 - protein, 549 aa, H1N1 swine influenza virus
  MISC_FEATURE (125)..(125): Xaa = N or D
DTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPXHXSNKGVTAACPHAGAKSFYKNLIWLVKKGNSYPKLSKSYINDKGKEVLVLWGIHHPSTSADQQSLYQNADAYVFVGSSRYSKXFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 2 - protein, 549 aa, H1N1 swine influenza virus
DTLCIGYHANNSTDTVDTVLEKNVTVTHSVNLLEDKHNGKLCKLRGVAPLHLGKCNIAGWILGNPECESLSTASSWSYIVETSSSDNGTCYPGDFIDYEELREQLSSVSSFERFEIFPKTSSWPDHESNKGVTAACPHAGAKSFYKNLIWLVKKGNSYPKLSKSYINDKGKEVLVLWGIHHPSTSADQQSLYQNADAYVFVGTSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 3 - protein, 549 aa, H1N1 swine influenza virus
  MISC_FEATURE (124)..(124): Xaa = P or L
DTLCIGYHANNSTDTVDTVLEKNVTVTHSVNLLEDKHNGKLCKLRGVAPLHLGKCNIAGWILGNPECESLSTASSWSYIVETSSSDNGTCYPGDFINYEELREQLSSVSSFERFEIFPKTSSWXXHXSNKGVTAACPHAGAKSFYKNLIWLVKKGNSYPKLSKSYINDKGKEVLVLWGIHHPSTSADQQSLYQNADAYVFVGTSRYSKXFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 4 - protein, 549 aa, H1N1 swine influenza virus
  MISC_FEATURE (125)..(125): Xaa = N or D
DTLCIGYHANNSTDTVDTVLEKNVTVTHSVNLLEDKHNGKLCKLRGVAPLHLGKCNIAGWILGNPECESLSTASSWSYIVETSSSDNGTCYPGDFINYEELREQLSSVSSFERFEIFPKTSSWPXHXSNKGVTAACPHAGAKSFYKNLIWLVKKGNSYPKLSKSYINDKGKEVLVLWGIHHPSTSADQQSLYQNADAYVFVGTSKYSKXFKPEIAVRPKVRDQEGRMNYYWTLVEPGDKITFEATGNLLVPRYAFAMERNAGSGIIISDTPVHDCNTTCQTPEGAINTSLPFQNIHPITLGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 5 - protein, 519 aa, H1N1 swine influenza virus
MNPNQKIITIGSVCMTIGMANLILQIGNIISIWISHSIQLGNQNQIETCNMNPNQKIITISSVCMTIGMANLILQIGNIISIWISHSIQLGNQNQIETCNQSVITYENNTWVNQTYVNISNTNFAAGQSVVSVKLAGNSSLCPVSGWAIYSKDNSVRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSNGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 6 - protein, 469 aa, H1N1 swine influenza virus
MNPNQKIITISSVCMTIGMANLILQIGNIISIWISHSIQLGNQNQIETCNQSVITYENNTWVNQTYVNISNTNFAAGQSVVSVKLAGNSSLCPVSGWAIYSKDNSIRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTVMTDGPSDGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 7 - protein, 469 aa, H1N1 swine influenza virus
MNPNQKIITIGSVCITIGMANLILQIGNIISIWISHSIQLGNQNQIETCNQSVITYENNTWVNQTYVNISNTNFAAGQSVVSVKLAGNSSLCPVSGWAIYSKDNSIRIGSKGDVFVIREPFISCSPLECRTFFLTQGALLNDKHSNGTIKDRSPYRTLMSCPIGEVPSPYNSRFESVAWSASACHDGISWLTIGISGPDNGAVAVLKYNGIITDTIKSWRNNILRTQESECACVNGSCFTVMTDGPSDGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGINEWSGYSGSFVQHPELTGLDCIKPCFWVELIRGRPKENTIWTSGSSISFCGVNSDTVGWSWPDGAELPFTIDK

SEQ ID NO: 8 - protein, 469 aa, H1N1 swine influenza virus
  MISC_FEATURE (222)..(222): Xaa = S or N
MNPNQKIITIGSVCMTIGMANLILQIGNIISIWISHSIQLGNQSQIETCNQSVITYENNTWVNQTYVNISNTNFAAGQSVVSVKLAGNSSLCPVSGWAIYSKDNSIRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNXILRTQESECACVNGSCFTXMTDGPSDGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRXGFEMIWDPNGWTGTDNNFSIKQDIVGINEWSGYSGSFVQHPELTGLDCIRPCFWVELIRGRPKENTIWTSGSSMSFCGVNSDTVGWSWPDGAELPFTIDK

SEQ ID NO: 9 - DNA, 1772 bp, H1N1 swine influenza virus
agcaaaagcaggggaaaacaaaagcaacaaaaatgaaggcaatactagtagttctgctatatacatttgcaaccgcaaatgcagacacattatgtataggttatcatgcgaacaattcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacacctagttcagacaatggaacgtgttacccaggagatttcatcgattatgaggagctaagagagcaattgagctcagtgtcatcatttgaaaggtttgagatattccccaagacaagttcatggcccaatcatgactcgaacaaaggtgtaacggcagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccatctactagtgctgaccaacaaagtctctatcagaatgcagatgcatatgtttttgtggggtcatcaagatacagcaagaagttcaagccggaaatagcaataagacccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcaaacacccaagggtgctataaacaccagcctcccatttcagaatatacatccgatcacaattggaaaatgtccaaaatatgtaaaaagcacaaaattgagactggccacaggattgaggaatatcccgtctattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacgagattactaacaaagtaaattctgttattgaaaagatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaatgtgaagaacttatatgaaaaggtaagaagccagctaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaactgtcgccagttcattggtactggtagtctccctgggggcaatcagtttctggatgtgctctaatgggtctctacagtgtagaatatgtatttaacattaggatttcagaagcatgagaaaaacacccttgttt

SEQ ID NO: 10 - DNA, 1698 bp, H1N1 swine influenza virus
atgaaggcaatactagtagttctgctatatacatttgcaaccgcaaatgcagacacattatgtataggttatcatgcgaacaattcaacagacactgtagacacagtactagaaaagaatgtaacagtaacacactctgttaaccttctagaagacaagcataacgggaaattatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcgattatgaggagctaagagaacaattgagctcagtgtcatcatttgaaaggtttgagatattccccaagacaagttcatggcccgatcatgaatcgaacaaaggtgtaacggcagcatgtcctcatgctggagcaaaaagcttctacaaaaatttaatatggctagttaaaaaaggaaattcatacccaaagctcagcaaatcctacattaatgataaagggaaagaagtcctcgtgctatggggcattcaccatccatctactagtgctgaccaacaaagtctctatcagaatgcagatgcatatgtttttgtggggacatcaagatacagcaagaagttcaagccggaaatagcaataagacccaaagtgagggatcaagaagggagaatgaactattactggacactagtagagccgggagacaaaataacattcgaagcaactggaaatctagtggtaccgagatatgcattcgcaatggaaagaaatgctggatctggtattatcatttcagatacaccagtccacgattgcaatacaacttgtcagacacccaagggtgctataaacaccagcctcccatttcagaatatacatccgatcacaattggaaaatgtccaaaatatgtaaaaagcacaaaattgagactggccacaggattgaggaatgtcccgtctattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacaagattactaacaaagtaaattctgttattgaaaagatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaatgtgaagaacttatatgaaaaggtaagaagccagttaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaactgtcgccagttcattggtactggtagtctccctgggggcaatcagtttctggatgtgctctaatgggtctctacagtgtagaatatgtatt

SEQ ID NO: 11 - DNA, 1701 bp, H1N1 swine influenza virus
atgaaggcaatactagtagttctgctatatacatttgcaaccgcaaatgcagacacattatgtataggttatcatgcgaacaattcaacagacactgtagacacagtactagaaaagaatgtaacagtaacacactctgttaaccttctagaagacaagcataacgggaaactatgcaaactaagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcaattatgaggagctaagagagcaattgagctcagtgtcatcatttgaaaggtttgagatattccccaagacaagttcatggcccaatcatgactcgaacaaaggtgtaacggcagcatgtcctcatgctggagcaaaaagcttctacaaaaatttaatatggctagttaaaaaaggaaattcatacccaaagctcagcaaatcctacattaatgataaagggaaagaagtcctcgtactatggggcattcaccatccatctactagtgctgaccaacaaagtctctatcagaatgcagatgcatatgtttttgtggggacatcaagatacagcaagaagttcaagccggaaatagcaataagacccaaagtgagggatcaagaagggagaatgaactattactggacactagtagagccgggagacaaaataacattcgaagcaactggaaatctagtggtaccgagatatgcattcgcaatggaaagaaatgctggatctggtattatcatctcagatacaccagtccacgattgcaatacaacttgtcagacacccaagggtgctataaacaccagcctcccatttcagaatatacatccgatcacaattggaaaatgtccaaaatatgtaaaaagcacaaaattgagactggccacaggattgaggaatgtcccgtctattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacaagattactaacaaagtaaattctgttattgaaaagatgaatacacagttcacagcagtaggtaaagagttcaaccacctggaaaaaagaatagagaatttaaataaaaaagttgatgatggtttcctggacatttggacttacaatgccgaactgttggttctattggaaaatgaaagaactttggactaccacgattcaaatgtgaagaacttatatgaaaaggtaagaagccagttaaaaaacaatgccaaggaaattggaaacggctgctttgaattttaccacaaatgcgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaactgtcgccagttcattggtactggtagtctccctgggggcaatcagtttctggatgtgctctaatgggtctctacagtgtagaatatgtatttaa

SEQ ID NO: 12 - DNA, 1706 bp, H1N1 swine influenza virus
gacacattatgtataggttatcatgcgaacaattcaacagacactgtagacacagtactagaaaagaatgtaacagtaacacactctgttaaccttctagaagacaagcataacgggaaactatgcaaactgagaggggtagccccattgcatttgggtaaatgtaacattgctggctggatcctgggaaatccagagtgtgaatcactctccacagcaagctcatggtcctacattgtggaaacatctagttcagacaatggaacgtgttacccaggagatttcatcaattatgaggagctaagagagcaattgagctcagtgtcatcatttgaaaggtttgagatattccccaagacaagttcatggcccaatcatgactcgaacaaaggtgtaacggcagcatgtcctcatgctggagcaaaaagcttctacaaaaatttaatatggctagttaaaaaaggaaattcatacccaaagctcagcaaatcctacattaacgataaagggaaagaagtcctcgtgctgtggggaattcaccatccatctactagtgctgaccaacaaagtctctatcagaatgcagatgcatatgtttttgtggggacatcaaaatacagcaagaaattcaagccggaaatagcagtaagacccaaagtgagggatcaagaagggagaatgaactattactggacactagtagagccgggagacaaaataacattcgaagcaactggaaatctattggtaccgagatatgcattcgcaatggaaagaaatgctggatctggtattatcatttcagatacaccagtccacgattgcaatacaacttgtcaaacacccgagggtgctataaacaccagcctcccatttcagaatatacatccgatcacacttggaaaatgtccaaaatatgtaaaaagcacaaaattgagactggccacaggattgaggaatgtcccgtctattcaatctagaggcctatttggggccattgccggtttcattgaaggggggtggacagggatggtagatggatggtacggttatcaccatcaaaatgagcaggggtcaggatatgcagccgacctgaagagcacacagaatgccattgacaagattactaacaaagtaaattctgttattgaaaagatgaatacacagttcacagcagtaggtaaagagttcaaccacctggaaaaaagaatagagaatttaaataaaaaggttgatgatggtttcctggacatttggacttacaatgccgaactgttggttctattggaaaatgaaagaactttggactaccacgattcaaatgtgaaaaacttatatgaaaaggtaagaagccagttaaaaaacaatgccaaagaaattggaaacggctgctttgaattttaccacaaatgcgataacacgtgcatggaaagtgtcaaaaatgggacttatgactacccaaaatactcagaggaagcaaaattaaacagagaagaaatagatggggtaaagctggaatcaacaaggatttaccagattttggcgatctattcaactgtcgccagttcattggtactggtagtctccctgggggcaatcagtttctggatgtgctctaatgggtctctacagtgtagaatatgtatttaacattaggatttcagaagcatgagaaaaacacccttgtttctactaatacgaggcag

SEQ ID NO: 13 - DNA, 1410 bp, H1N1 swine influenza virus
atgaatccaaaccaaaagataataaccattggttcggtctgtataacaattggaatggctaacttaatattacaaattggaaacataatctcaatatggattagccactcaattcaacttgggaatcaaaatcagattgaaacatgcaatcaaagcgtcattacttatgaaaacaacacttgggtaaatcagacatatgttaacatcagcaacaccaactttgctgctggacagtcagtggtttccgtgaaattagcgggcaattcctctctctgccctgttagtggatgggctatatacagtaaagacaacagtataagaatcggttccaagggggatgtgtttgtcataagggaaccattcatatcatgctcccccttggaatgcagaaccttcttcttgactcaaggggccttgctaaatgacaaacattccaatggaaccattaaagacaggagcccatatcgaaccctaatgagctgtcctattggtgaagttccctctccatacaactcaagatttgagtcagtcgcttggtcagcaagtgcttgtcatgatggcatcagttggctaacaattggaatttctggcccagacaatggggcagtggctgtgttaaagtacaacggcataataacagacactatcaagagttggagaaacaatatattgagaacacaagagtctgaatgtgcatgtgtaaatggttcttgttttactgtaatgaccgatggaccaagtgatggacaggcctcatacaagatcttcagaatagaaaagggaaagatagtcaaatcagtcgaaatgaatgcccctaattatcactatgaggaatgctcctgttatcctgattctagtgaaatcacatgtgtgtgcagggataactggcatggctcgaatcgaccgtgggtgtctttcaaccagaatctggaatatcagataggatacatatgcagtgggattttcggagacaatccacgccctaatgataagacaggcagttgtggtccagtatcgtctaatggagcaaatggagtaaaaggattttcattcaaatacggcaatggtgtttggatagggagaactaaaagcattagttcaagaaacggttttgagatgatttgggatccgaacggatggactgggacagacaataacttctcaataaagcaagatatcgtaggaataaatgagtggtcaggatatagcgggagttttgttcagcatccagaactaacagggctggattgtataaaaccttgcttctgggttgaactaatcagagggcgacccaaagagaacacaatctggactagcgggagcagcatatccttttgtggtgtaaacagtgacactgtgggttggtcttggccagacggtgctgagttgccatttaccattgacaagtaa

SEQ ID NO: 14 - DNA, 1410 bp, H1N1 swine influenza virus
atgaatccaaaccaaaagataataaccattagttcggtctgtatgacaattggaatggctaacttaatattacaaattggaaacataatctcaatatggattagccactcaattcaacttgggaatcaaaatcagattgaaacatgcaatcaaagcgtcattacttatgaaaacaacacttgggtaaatcagacatatgttaacatcagcaacaccaactttgctgctggacagtcagtggtttccgtgaaattagcgggcaattcctctctctgccctgttagtggatgggctatatacagtaaagacaacagtataagaatcggttccaagggggatgtgtttgtcataagggaaccattcatatcatgctcccccttggaatgcagaaccttcttcttgactcaaggggccttgctaaatgacaaacattccaatggaaccattaaagacaggagcccatatcgaaccctaatgagctgtcctattggtgaagttccctctccatacaactcaagatttgagtcagtcgcttggtcagcaagtgcttgtcatgatggcatcaattggctaacaattggaatttctggcccagacaatggggcagtggctgtgttaaagtacaacggcataataacagacactatcaagagttggagaaacaatatattgagaacacaagagtctgaatgtgcatgtgtaaatggttcttgctttactgtaatgaccgatggaccaagtgatggacaggcctcatacaagatcttcagaatagaaaagggaaagatagtcaaatcagtcgaaatgaatgcccctaattatcactatgaggaatgctcctgttatcctgattctagtgaaatcacatgtgtgtgcagggataactggcatggctcgaatcgaccgtgggtgtctttcaaccagaatctggaatatcagataggatacatatgcagtgggattttcggagacaatccacgccctaatgataagacaggcagttgtggtccagtatcgtctaatggagcaaatggagtaaaaggattttcattcaaatacggcaatggtgtttggatagggagaactaaaagcattagttcaagaaacggttttgagatgatttgggatccgaacggatggactgggacagacaataacttctcaataaagcaagatatcgtaggaataaatgagtggtcaggatatagcgggagttttgttcagcatccagaactaacagggctggattgtataagaccttgcttctgggttgaactaatcagagggcgacccaaagagaacacaatctggactagcgggagcagcatatccttttgtggtgtaaacagtgacactgtgggttggtcttggccagacggtgctgagttgccatttaccattgacaagtaa

SEQ ID NO: 15 - DNA, 1427 bp, H1N1 swine influenza virus
atgaatccaaaccaaaagataataaccattggttcggtctgtatgacaattggaatggctaacttaatattacaaattggaaacataatctcaatatggattagccactcaattcaacttgggaatcaaagtcagattgaaacatgtaatcaaagcgtcattacttatgaaaacaacacttgggtaaatcagacatatgttaacatcagcaacaccaactttgctgctggacagtcagtggtttccgtgaaattagcgggcaattcctctctctgccctgttagtggatgggctatatacagtaaagacaacagtataagaatcggttccaagggggatgtgtttgtcataagggaaccattcatatcatgctcccccttggaatgcagaaccttcttcttgactcaaggggccttgctaaatgacaaacattccaatggaaccattaaagacaggagcccatatcgaaccctaatgagctgtcctattggtgaagttccctctccatacaactcaagatttgagtcagtcgcttggtcagcaagtgcttgtcatgatggcatcaattggctaacaattggaatttctggcccagacaatggggcagtggctgtgttaaagtacaacggcataataacagacactatcaagagttggagaaacagtatattgagaacacaagagtctgaatgtgcatgtgtaaatggttcttgctttaccataatgaccgatggaccaagtgatggacaggcctcatacaagatcttcagaatagaaaagggaaaaatagtcaaatcagtcgaaatgaatgcccctaattatcactatgaggaatgctcctgttatcctgattctagtgaaatcacttgtgtgtgcagggataactggcatggctcgaatcgaccgtgggtgtctttcaaccagaatctggaataccagataggatacatatgcagtgggattttcggagacaatccacgccctaatgataagacaggcagttgtggtccagtatcgtctaatggagcaaatggagtaaaaggattttcattcaaatacggcaatggtgtttggatagggagaactaaaagcattagttcaagaaaaggttttgagatgatttgggatccaaacggatggactgggacagacaataacttctcaataaagcaagatatcgtaggaataaatgagtggtcaggatatagcgggagttttgttcagcatccagaactaacagggctggattgtataagaccttgcttctgggttgaactaatcagagggcgacccaaagagaacacaatctggactagcgggagcagcatgtccttttgtggtgtaaacagtgacactgtgggttggtcttggccagacggtgctgagttgccatttaccattgacaagtaatttgttcaaaaaactcc

SEQ ID NO: 16 - DNA, 1458 bp, H1N1 swine influenza virus
agcaaaagcaggagtttaaaatgaatccaaaccaaaagataataaccattggttcggtctgtatgacaattggaatggctaacttaatattacaaattggaaacataatctcaatatggattagccactcaattcaacttgggaatcaaaatcagattgaaacatgcaatcaaagcgtcattacttatgaaaacaacacttgggtaaatcagacatatgttaacatcagcaacaccaactttgctgctggacagtcagtggtttccgtgaaattagcgggcaattcctctctctgccctgttagtggatgggctatatacagtaaagacaacagtgtaagaatcggttccaagggggatgtgtttgtcataagggaaccattcatatcatgctcccccttggaatgcagaaccttcttcttgactcaaggggccttgctaaatgacaaacattccaatggaaccattaaagacaggagcccatatcgaaccctaatgagctgtcctattggtgaagttccctctccatacaactcaagatttgagtcagtcgcttggtcagcaagtgcttgtcatgatggcatcaattggctaacaattggaatttctggcccagacaatggggcagtggctgtgttaaagtacaacggcataataacagacactatcaagagttggagaaacaatatattgagaacacaagagtctgaatgtgcatgtgtaaatggttcttgctttactgtaatgaccgatggaccaagtaatggacaggcctcatacaagatcttcagaatagaaaagggaaagatagtcaaatcagtcgaaatgaatgcccctaattatcactatgaggaatgctcctgttatcctgattctagtgaaatcacatgtgtgtgcagggataactggcatggctcgaatcgaccgtgggtgtctttcaaccagaatctggaatatcagataggatacatatgcagtgggattttcggagacaatccacgccctaatgataagacaggcagttgtggtccagtatcgtctaatggagcaaatggagtaaaagggttttcattcaaatacggcaatggtgtttggatagggagaactaaaagcattagttcaagaaacggttttgagatgatttgggatccgaacggatggactgggacagacaataacttctcaataaagcaagatatcgtaggaataaatgagtggtcaggatatagcgggagttttgttcagcatccagaactaacagggctggattgtataagaccttgcttctgggttgaactaatcagagggcgacccaaagagaacacaatctggactagcgggagcagcatatccttttgtggtgtaaacagtgacactgtgggttggtcttggccagacggtgctgagttgccatttaccattgacaagtaatttgttcaaaaaactccttgtttctact